(19) United States
(12) Patent Application Publication
(10) Pub. No.: US 2026/0022368 A1
(43) Pub. Date: Jan. 22, 2026

(54) SYSTEMS AND METHODS FOR VARIANT DETECTION IN CELLS

(71) Applicant: University of Washington, Seattle, WA (US)

(72) Inventors: Scott R. Kennedy, Seattle, WA (US); Georg Seelig, Seattle, WA (US); Ian T. Dowsett, Seattle, WA (US); Matthew S. Hirano, Seattle, WA (US)

(73) Assignee: University of Washington, Seattle, WA (US)

(21) Appl. No.: 18/993,487

(22) PCT Filed: Jul. 12, 2023
(86) PCT No.: PCT/US2023/070069

§ 371 Date: Jan. 10, 2025

Related U.S. Application Data
(60) Provisional application No. 63/388,370, filed on Jul. 12, 2022.

(51) Int. Cl. C12N 15/10; C12Q 1/6886

(52) U.S. Cl. CPC C12N 15/1065; C12Q 1/6886; C12Q 2600/156

(57) ABSTRACT

Systems, devices, kits, and methods for generating sequencing libraries for detecting low-frequency variants, and for distinguishing low-frequency biological variants from low-frequency technical variants, with duplex sequencing. Methods include introducing multiple levels of indices to cellular nucleic acid molecules of cells of a biological sample to enable differentiation of a cell from other cells of the sample, differentiation of a double-stranded cellular nucleic acid molecule from others of the cell, and differentiation of the individual strands of the double-stranded cellular nucleic acid molecule. Technical variants introduced during the workflow are identified as such due to their occurrence in only one family of duplex strands of the sequencing library, and biological variants are detected due to their occurrence in both families of duplex strands of the sequencing library. Detected biological variants can be linked with a particular cell of the biological sample in a high-throughput manner.

### CROSS-REFERENCE(S) TO RELATED APPLICATION(S)

This application claims the benefit of U.S. Provisional Patent Application No. 63/388,370, filed Jul. 12, 2022, the disclosure of which is hereby incorporated by reference in its entirety.

### STATEMENT OF GOVERNMENT LICENSE RIGHTS

This invention was made with government support under Grant No. 1R21HG011229-01, awarded by the National Human Genome Research Institute. Grant No. 2017-DN-BX-0160, awarded by the National Institute of Justice, Grant No. T32 AG066574, awarded by the National Institute on Aging, Grant No. W911NF-15-2-0127, awarded by the U.S. Army, and Grant No. W911NF-17-1-0158, awarded by the U.S. Army Research Office. The government has certain rights in the invention.

### STATEMENT REGARDING SEQUENCE LISTING

The sequence listing associated with this application is provided in XML format in lieu of a paper copy and is hereby incorporated by reference into the specification. The name of the XML file containing the sequence listing is 3915-P1306WO-UW_Sequence_Listing_ST-26.xml. The XML file is 14 KB; was created on Jul. 12, 2023; and is being submitted via Patent Center with the filing of the specification.

### BACKGROUND

Somatic mutations cause changes in the sequence of a cell's DNA. These changes, in turn, can alter how cells function. There is significant interest in understanding how somatic mutations, especially ones associated with cancer, affect cell function. More importantly, it is becoming increasingly important to determine if non-cancerous cells that harbor specific cancer-associated mutations are functionally impaired or differ from normal cells. Unfortunately, this analysis can only be performed at the single-cell level. Developed methods have allowed for the simultaneous sequencing of both RNA and DNA-derived nucleic acids at the single cell level, but none have been able to relate the transcriptomic data to the presence of genomic variants in a high throughput manner.

The recent invention of SPLIT-Seq. which uses split-pool cellular barcoding, provides the ability to uniquely identify sequencing data derived from the same individual cell in a massively parallel manner. The technique is for labeling cellular molecules within cells of a biological sample, e.g., as described in U.S. Pat. No. 11,427,856 B2, which is incorporated by reference herein in its entirety for all purposes. In the technique, molecules within cells, such as RNAs, cDNAs, DNAs, proteins, peptides, and/or antigens, are uniquely labeled on a cell basis by a population splitting/tagging/pooling procedure that is iteratively performed to provide XY labeling options, wherein X is the number of aliquots per split and Y is the number of splits. Briefly, the technique can include labeling nucleic acids in a first cell. The method can include; (a) generating complementary DNAs (cDNAs) within a plurality of cells comprising the first cell by reverse transcribing RNAs using a reverse transcription primer comprising a 5′ overhang sequence; (b) dividing the plurality of cells into a number (n) of aliquots; (c) providing a plurality of nucleic acid tags to each of the n aliquots, wherein each labeling sequence of the plurality of nucleic acid tags provided into a given aliquot is the same, and wherein a different labeling sequence is provided into each of the n aliquots; (d) binding at least one of the cDNAs in each of the n aliquots to the nucleic acid tags; (e) combining the n aliquots; and (f) repeating steps (b), (c), (d), and (e) with the combined aliquot. In at least some instances, step (f) (i.e., steps (b). (c). (d), and (e)) can be repeated a number of times sufficient to generate a unique series of labeling sequences for the cDNAs in the first cell. Stated another way, step (f) can be repeated a number of times such that the cDNAs in the first cell can have a first unique series of labeling sequences, the cDNAs in a second cell can have a second unique series of labeling sequences, the cDNAs in a third cell can have a third unique series of labeling sequences, and so on. The method can provide for the labeling of cDNA sequences from single cells with unique barcodes, wherein the unique barcodes can identify or aid in identifying the cell from which the cDNA originated. At the conclusion of such a procedure, cellular molecules are labeled, and individual sequences can be linked back to an individual cell. Specifically, barcoded cDNA can be mixed together and sequenced (e.g., using Next-Generation Sequencing (NGS)), such that data can be gathered regarding RNA expression at the level of a single cell. While this approach solves the throughput problem, the high error rate of current sequencing platforms precludes the ability detect somatic mutations, which are extremely rare compared to ‘wild-type’ sequences.

While such cell molecule labeling techniques can be used for associating variants with cells, they are nevertheless subject to sequencing errors that occur during NGS workflows. NGS approaches have a relatively high error rate; on the order of one erroneous base call per 100-1.000 sequenced nucleotides. Although this error rate is acceptable for studying inherited germline mutations, it greatly limits the analysis of subclonal mutations, or mutations that are present in only a fraction of cells within a population. Example sources of sequencing errors include sample handling, library preparation, enrichment polymerase chain reaction (PCR), and sequencing. The presence of these technical variants in the sequencing dataset confounds efforts to investigate biological variants, especially low-frequency biological variants, whose contributions to disease states can be complex.

Duplex Sequencing (DS) is a NGS methodology capable of detecting a single mutation among more than 1×10⁷ wild-type nucleotides, and enables the study of heterogeneous populations and very-low-frequency genetic variants. DS can be applied to any double-stranded DNA sample and relies on the ligation of sequencing adapters harboring random yet complementary double stranded nucleotide sequences to the sample DNA of interest. Individually labeled strands are then PCR-amplified, creating sequence ‘families’ that share a common tag sequence derived from the two original complementary strands. Mutations are scored only if the variant is present in the PCR families arising from both of the two DNA strands, that is, if they correspond to biological variants. Mutations introduced during PCR by DNA polymerase misincorporations or arising from DNA damage will appear in only one of the two DNA strands and thus are considered technical variants and not biological variants. In brief, sequencing library construction for DS is similar to the IlluminaR library preparation protocol. The protocol follows the steps of DNA shearing by sonication, size selection, end repair, 3′ dA-tailing, adapter ligation, PCR amplification and, optionally, targeted DNA capture. However, with DS, the sequencing adapters are re-designed to allow for more efficient dA-tailing of the sample DNA, and the adapters require a different synthesis method. Additional features of DS are described in Kennedy S. R, et al., Nat Protoc. 2014 Nov.; 9 (11); 2586 2606, which is incorporated by reference herein in its entirety for all purposes.

While transcript, transcriptomic, genetic, and genomic biological variants are all of significant interest to a variety of fields, including but not limited to cancer research and diagnostics, there is currently no satisfactory approach for sequencing polynucleotides of a large number of cells in a high-throughput manner with a high degree of accuracy for detection of low-frequency biological variants and mapping of such variants to individual cells. The present disclosure addresses this and other long-felt and unmet needs in the art.

### SUMMARY

This summary is provided to introduce a selection of concepts in a simplified form that are further described below in the Detailed Description. This summary is not intended to be used as an aid in determining the scope of the claimed subject matter.

The present technology relates generally to methods for detecting rare variants in nucleic acid sequences in a cell-specific or cell-identified manner. In embodiments, the methods provide for single cell analysis and interrogation, and associated reagents for use in such methods. In particular, embodiments of the technology are directed to utilizing duplex sequencing for generating high accuracy, error-corrected sequence reads attributable to a particular cell or cell type. In embodiments, the methods provide transcriptome information for assessing a cell type or cell origin and provide variant detection, including rare variant detection, that can be associated with the transcriptome information for any particular cell. In embodiments, combinations of variants can be identified and attributed to a particular cell or cell type. Applications of cell specific variant detection (alone or in combination with a plurality of variants) include, but are not limited to, disease detection, disease-state assessment, disease risk assessment, cancer detection including early cancer detection, measurable residual disease (MRD) detection, identifying a cancer risk, identifying a treatment-resistant clone, identifying genotoxic or carcinogenic agents. monitoring cell therapy treatment, monitoring transplant rejection, etc.

In embodiments, provided methods and compositions allow for the detection of mutations in a population of cells within a biological sample, wherein the mutations are associated with a specific cell type or a particular cell. Accordingly, aspects of the present technology can interrogate multiple cells in a high throughput manner and provide both RNA (e.g., transcriptome) and DNA derived sequencing information with a high level of sensitivity and specificity. In embodiments, the number of cells that can be interrogated by the methods and systems disclosed herein can be in the 10s, the 100s, the 1000s, the 10,000s, the 100,000s, or in the 1,000,000s.

Certain aspects of the present technology provide methods for preparation of next generation sequence (NGS) library preparation for duplex sequencing, wherein the library is partially prepared in vivo. Further aspects of the present technology provide methods for high throughput single cell analysis of nucleic acid material, including but not limited to transcriptome analysis, genomic DNA variant detection and phased variant detection with high sensitivity and specificity.

In an aspect, the disclosure provides a method for preparing a sequencing library for duplex sequencing, the method comprising; a) introducing a sample index to cellular nucleic acid molecules of cells of a biological sample; b) introducing a cell index to the cells according to a method of cell indexing, the method of cell indexing comprising; a.dividing an initial pool of the cells into a plurality of aliquots, wherein cell membranes of the cells retain cellular nucleic acid molecules therein; b, contacting an aliquot of the plurality of aliquots with a corresponding aliquot-specific barcode nucleic acid such that the cellular nucleic acid molecules of the aliquot are ligated to the corresponding aliquot-specific barcode nucleic acid to form aliquot-barcoded cellular nucleic acid molecules within cells of the aliquot; c, pooling the plurality of aliquots of cells to form a subsequent pool, wherein the subsequent pool serves as the initial pool of the cells for a subsequent repetition of steps a., b . . , and c.; and d, repeating steps a., b . . , and c, until most cells or every cell of the biological sample contains cellular nucleic acid molecules therein that are uniquely labeled according to the cell index; c) introducing a molecular index as a unique molecular identifier (UMI) to the cellular nucleic acid molecules; and d) providing a strand index as a strand-defining element (SDE) to the cellular nucleic acid molecules; wherein the sample index is for identification of a particular biological sample among biological samples, the cell index is for identification of a particular cell among cells, the molecular index is for identification of a particular cellular nucleic acid molecule among cellular nucleic acid molecules, and the strand index is for identification of a particular strand of the particular cellular nucleic acid molecule.

In another aspect, the disclosure provides a method for preparing a sequencing library for duplex sequencing, the method comprising; a) covalently attaching, with a transposase, a barcode nucleic acid molecule to cellular nucleic acid molecules of cells of a biological sample to form barcoded cellular nucleic acid molecules; and b) introducing a strand index as a strand-defining element (SDE) to the cellular nucleic acid molecules.

In another aspect, the disclosure provides a method for preparing a sequencing library for duplex sequencing, the method comprising; a) covalently attaching, with a transposase, first and second sequencing adaptors to cellular nucleic acid molecules of cells of a biological sample to form adaptor-labeled cellular nucleic acid molecules; and b) introducing a strand index as a strand-defining element (SDE) to the cellular nucleic acid molecules, wherein the SDE is an orientation of the sequencing adaptors of the adaptor-labeled cellular nucleic acid molecules with respect to a 5′ end or a 3′ end of the adaptor-labeled cellular nucleic acid molecules.

In another aspect, the disclosure provides a method for preparing a sequencing library for duplex sequencing, the method comprising; a) covalently attaching. with a transposase, a first sequencing adaptor to cellular nucleic acid molecules of cells of a biological sample to form adaptor-labeled cellular nucleic acid molecules, wherein the first sequencing adaptor is attached to a single strand of a mosaic end (ME) element by a deoxyuracil (dU) residue; and b) introducing a strand index as a strand-defining element (SDE) to the cellular nucleic acid molecules, wherein the SDE is a distance from a barcode of the adaptor-labeled cellular nucleic acid molecules to a sequence of the cellular nucleic acid molecules.

In another aspect, the disclosure provides a method of generating an error-corrected sequence read of a double-stranded genomic DNA (gDNA) material in a cell-specific or cell-identifiable manner, the method comprising; accessing cellular nucleic acid material within cells and/or cellular organelles, wherein the cellular nucleic acid material comprises the double-stranded gDNA material and double-stranded cDNA material derived from RNA within the cells and/or cellular organelles; indexing the cellular nucleic acid material thereby forming indexed-target nucleic acid complexes having a first population of complexes comprising indexed-target gDNA complexes and a second population of complexes comprising indexed-target cDNA complexes, wherein each indexed-target nucleic acid complex in a plurality of the indexed-target nucleic acid complexes comprises (a) a cell index that identifies the target nucleic acid material as originating from a particular cell among a population of cells, and (b) a molecular index comprising a UMI sequence that identifies the indexed-target nucleic acid complex among the first and second populations of complexes; providing an SDE for the indexed-target nucleic acid complexes of the first population, wherein the SDE identifies a particular strand of a particular indexed-target nucleic acid complex; for one or more indexed-target nucleic acid complexes in the first population-amplifying each strand of the indexed-target nucleic acid complex to produce a plurality of first strand amplicons and a plurality of second strand amplicons; sequencing one or more of the first strand amplicons and one or more of the second strand amplicons to produce one or more first strand sequence reads and one or more second strand sequence reads; confirming the presence of at least one first strand sequence read and at least one second strand sequence read; and comparing the at least one first strand sequence read with the at least one second strand sequence read and generating an error-corrected sequence read of the double-stranded target nucleic acid material by—discounting nucleotide positions that do not agree; or removing compared first and second strand sequence reads having one or more nucleotide positions where the compared first and second strand sequence reads are non-complementary.

In another aspect, the disclosure provides a method of identifying a DNA variant in a cell within a population of cells, the method comprising; providing a population of cells from a biological sample; accessing cellular nucleic acid material within cells of the population, wherein the cellular nucleic acid material comprises double-stranded gDNA material and single-stranded RNA material within the cells; indexing the double-stranded gDNA material to generate indexed-target gDNA complexes, wherein each indexed-target gDNA complex in a plurality of the indexed-target gDNA complexes comprises (a) a cell index that identifies the target gDNA material as originating from a particular cell among the population of cells, and (b) a UMI that identifies the indexed-target gDNA complex among the plurality of the indexed-target gDNA complexes; providing an SDE for the indexed-target gDNA complexes, wherein the SDE identifies a particular strand of a particular indexed-target gDNA complex; reverse transcribing the single-stranded RNA material within the cells to generate double stranded cDNA material; indexing the double-stranded cDNA material to generate indexed-target cDNA complexes, wherein each indexed-target cDNA complex in a plurality of the indexed-target cDNA complexes comprises (a) the cell index that identifies the target cDNA material originating from the same particular cell among the population of cells, and (b) a UMI that identifies the indexed-target cDNA complex among the plurality of the indexed-target cDNA complexes and indexed-target gDNA complexes; for one or more indexed-target gDNA complexes in the plurality of the indexed-target gDNA complexes-amplifying each of a first strand and a second strand of the indexed-target gDNA complex, resulting in each strand generating a distinct, yet related, set of amplified indexed-target gDNA products; sequencing each of a plurality of first strand indexed-target gDNA products and a plurality of second strand indexed-target nucleic acid products; confirming the presence of at least one sequence read from each strand of the indexed-target gDNA complex; and comparing the at least one sequence read obtained from the first strand with the at least one sequence read obtained from the second strand to form a consensus sequence read of the indexed-target gDNA complex having only nucleotide bases at which the sequence of both strands of the indexed-target gDNA complex are in agreement, such that a variant occurring at a particular position in the consensus sequence read is identified as a true DNA variant.

In another aspect, the disclosure provides a kit configured for error corrected duplex sequencing of double-stranded nucleic acids to characterize a cell within a population of cells, the kit comprising at least one set of combinatorial cell indexing oligonucleotides, wherein at least a subset of the oligonucleotides comprises a UMI and an SDE for error corrected duplex sequencing.

In another aspect, the disclosure provides a non-transitory computer-readable storage medium having instructions stored thereon which, when executed by at least one processor, cause the at least one processor to perform a method for providing duplex sequencing data for double-stranded nucleic acid molecules in a cell from a biological sample, the method comprising; receiving raw sequence data from a user computing device; creating a sample-specific data set comprising a plurality of raw sequence reads derived from a plurality of nucleic acid molecules in the sample; grouping sequence reads from families representing an original double-stranded nucleic acid molecule, wherein the grouping is based on a shared unique identifier (UMI) sequence; comparing a first strand sequence read and a second strand sequence read from an original double-stranded nucleic acid molecule to identify one or more correspondences between the first and second strand sequences reads; providing duplex sequencing data for the double-stranded nucleic acid molecules in the sample; and grouping duplex consensus sequences into cell families representing an original cell, wherein the grouping is based on a shared cell index sequence.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a flow diagram illustrating duplex sequencing (DS) method steps in accordance with aspects of the present technology.

FIGS. 2A-2B are flow diagrams illustrating single-cell duplex sequencing (single-cell DS) method steps in accordance with aspects of the present technology.

FIG. 3A is a conceptual illustration of a combinatorial cell indexing scheme for use in an embodiment of a single-cell DS method in accordance with an aspect of the present technology.

FIG. 3B is a conceptual illustration of a cell indexed gDNA molecule in accordance with aspects of the present technology.

FIGS. 3C-3D are conceptual illustrations of various single-cell DS method steps using the combinatorial cell indexing scheme of FIG. 3A, and in accordance with an embodiment of the present technology.

FIGS. 4A-4B are conceptual illustrations of various single-cell DS method steps using a combinatorial cell indexing scheme, and in accordance with another embodiment of the present technology. IIIIIIII=Sample index; NNNNNNNN=UMI; XXXXXXXX=Aliquot-specific barcode.

FIGS. 5A-5B are conceptual illustrations of various single-cell DS method steps utilizing a transposase for adaptor integration and in accordance with yet another embodiment of the present technology. XXX. . . . XXX-genomic DNA (gDNA).

FIGS. 6A-6B are conceptual illustrations of various single-cell DS method steps utilizing a transposase in accordance with another embodiment of the present technology. XXX. . . . XXX=genomic DNA (gDNA).

FIG. 7A illustrates nucleic acid sequencing adapter molecules for use with embodiments of the present technology.

FIGS. 7B-7C are conceptual illustrations of various single-cell DS method steps utilizing a transposase, the sequencing adapters of FIG. 7A, and a combinatorial cell indexing scheme, and in accordance with another embodiment of the present technology. Newly synthesized strand denoted by brackets.

FIG. 8 is a conceptual illustration showing an example mechanistic detail for utilizing adaptor ligation with a transposase, and in accordance with an embodiment of the present technology.

FIGS. 9A-9B are conceptual illustrations of various duplex sequencing (DS) library preparation steps utilizing a transposase and in accordance with an embodiment of the present technology. NNNNNNNN=UMI; XXXXXXXX=Sample Index.

FIGS. 10A-10D illustrate results of a single cell DS experiment in accordance with an embodiment of the present technology.

FIG. 11 is a flow diagram illustrating a routine for providing Duplex Sequencing Data for double-stranded nucleic acid molecules in a cell (e.g., a cell from a biological mixture) in accordance with an embodiment of the present technology.

FIG. 12 is a flow diagram illustrating a routine for detecting and identifying variant(s) in a cell in a cell population in accordance with an embodiment of the present technology.

TABLE 1
Select drawings numeral references and descriptions

| Numeral | |
|---|---|
| Reference | Description |
| 11 | Plurality of cells |
| 12 | Individual cell |
| 13 | Aliquot |
| 14 | Pooled cells |
| 15 | Split cells |
| 16 | Cell index (plurality of barcodes) |
| 17 | Genomic DNA (gDNA) |
| 18 | Strand-defining element (SDE) shown as |
| | base pair mismatch |
| 19 | Aliquot-specific barcode 1 |
| 20 | Aliquot-specific barcode 2 |
| 21 | Aliquot-specific barcode 3 |
| 21a | Unique molecular identifier (UMI) |
| 22 | Splinting oligonucleotide 1 |
| 23 | Splinting oligonucleotide 2 |
| 24 | Nucleosome |
| 25 | Cellular nucleic acid molecules and/or |
| | polynucleotide sequences (e.g., gDNA) |
| 25a | Cellular nucleic acid molecule strand 1 |
| 25b | Cellular nucleic acid molecule strand 2 |
| 26 | Platform-specific adaptor, e.g., Illumina ® |
| | adaptor (Index 2 or Index 1 adaptor; i7 or |
| | i5 adaptor) |
| 27 | Cell indexing adapter |
| 28a | Transposition site 1 |
| 28b | Transposition site 2 |
| 29a | Adapter molecule 1 |
| 29b | Adapter molecule 2 |
| 36 | Uracil containing element |
| 37a, 37b | Non-uracil containing element |
| 39 | Splinting oligonucleotide 3 |
| 40a | SDE primer 1 |
| 40b | SDE primer 2 |
| 41a | Primer 1 for platform-specific adaptor sequence, |
| | e.g., Illumina ® adaptor (Index 2 or Index 1 |
| | adaptor; i7 or i5 adaptor) |
| 41b | Primer 2 for platform-specific adaptor sequence, |
| | e.g., Illumina ® adaptor (Index 2 or Index 1 |
| | adaptor; i7 or i5 adaptor) |
| 42 | Sequencing adaptor 1 (SEQ ID NO: 2) |
| 43 | Sequencing adaptor 2 (SEQ ID NO: 1) |
| 50a, 50b | Barcode |
| 51a | Platform-specific adaptor sequence, e.g., |
| | Illumina ® adaptor (Index 2 or Index 1 |
| | adaptor; i7 or i5 adaptor) |
| 51b | Platform-specific adaptor sequence, e.g., |
| | Illumina ® adaptor (Index 2 or Index 1 |
| | adaptor; i7 or i5 adaptor) |
| 52a | Primer for polymerase extension |
| 52b | Primer for polymerase extension |
| 53a, 53b | Sample index |
| 54a, 54b | Platform-specific adaptor sequence, e.g., |
| | Illumina ® adaptor (Index 2 or Index 1 |
| | adaptor; i7 or i5 adaptor) |
| 58 | Illumina ® i7 sequencing adaptor 1, |
| | wherein n = deoxyuracil (dU) |
| | (SEQ ID NOs.: 5, 10, 11) |
| 59 | Sequencing adapter 1 |
| 60 | Tn5 transposase mosaic end (ME) element |
| 60a | Tn5 ME element strand 1 |
| 60b | Tn5 ME element strand 2 |
| 61a, 61b | Primer for polymerase extension |
| 62 | Illumina ® i5 sequencing adaptor 1 |
| | (SEQ ID NO: 3) |
| 63 | Illumina ® i7 sequencing adaptor 2 |
| | (SEQ ID NO: 4) |
| 64 | Sequencing adaptors |
| 65 | In-situ gDNA |
| 74 | UMI barcode |
| 75 | Illumina ® P5 graft adaptor primer |
| 76 | Illumina ® P7 graft adaptor primer |
| 84 | P5 flow cell graft sequence |
| 85 | P7 flow cell graft sequence |
| 86 | Wobble bases sequence 1 (SEQ ID NOs: 6-7) |
| 87 | Wobble bases sequences 2-3 (SEQ ID NOs: 8-11) |
| 88 | Wobble bases sequence 4 (SEQ ID NOs: 12-13) |

### DETAILED DESCRIPTION

The present technology is directed, at least in part, to methods for generating an error-corrected sequence read of a double-stranded genomic DNA (gDNA) material in a cell-specific or cell-identifiable manner.

The present disclosure encompasses a recognition that high fidelity sequencing techniques, such as Duplex Sequencing (DS), can be used to detect and/or quantify low frequency genetic variants in a cell-specific manner. For example, various embodiments of the present technology include performing DS methods for identifying one or more genetic variants among target double-stranded nucleic acid molecules and determining a variant frequency of the one or more variants. Further examples of the present technology are directed to methods for identifying a DNA variant in a cell within a population of cells. Various aspects of the present technology have many applications in both pre-clinical and clinical therapies as well as other industry-wide implications.

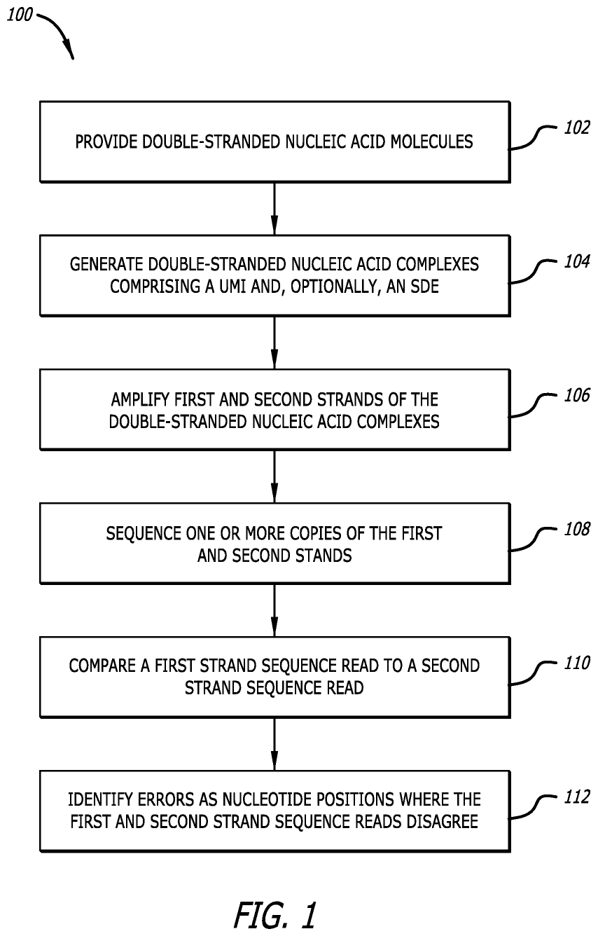

Specific details of several embodiments of the technology are described below with reference to the Drawings (e.g., FIGS. 1-12). Although many of the embodiments are described herein with respect to DS, other sequencing modalities capable of generating error-corrected sequencing reads, in addition to those described herein, are within the scope of the present technology. Additionally, other embodiments of the present technology can have different configurations, components, or procedures than those described herein. A person of ordinary skill in the art, therefore, will accordingly understand that the technology can have other embodiments with additional elements and that the technology can have other embodiments without several of the features shown and described below with reference to FIGS. 1-12.

### Certain Definitions

In order for the present disclosure to be more readily understood, certain terms are first defined below; Additional definitions for the following terms and other terms are set forth throughout the specification.

In this application, unless otherwise clear from context, the term “a” may be understood to mean “at least one.” As used in this application, the term “or” may be understood to mean “and/or” In this application, the terms “comprising” and “including” may be understood to encompass itemized components or steps whether presented by themselves or together with one or more additional components or steps. Where ranges are provided herein, the endpoints are included. As used in this application, the term “comprise” and variations of the term, such as “comprising” and “comprises,” are not intended to exclude other additives, components, integers or steps.

About; The term “about”, when used herein in reference to a value, refers to a value that is similar, in context to the referenced value. In general, those skilled in the art, familiar with the context. will appreciate the relevant degree of variance encompassed by “about” in that context. For example, in embodiments, the term “about” may encompass a range of values that are within 25%, 20%, 19%, 18%, 17%, 16%. 15%, 14%. 13%, 12%, 11%, 10%, 9%, 8%, 7%, 6%, 5%, 4%, 3%. 2%, 1%, or less of the referred value.

Biological Sample; As used herein, the term “biological sample” or “sample” typically refers to a sample obtained or derived from a biological source (e.g., a tissue or organism or cell culture) of interest, as described herein. In embodiments, a source of interest comprises an organism, such as an animal or human. In other embodiments, a source of interest comprises a microorganism, such as a bacterium, virus, protozoan, or fungus. In further embodiments, a source of interest may be a synthetic tissue, organism, cell culture, nucleic acid or other material. In yet further embodiments, a source of interest may be a plant-based organism. In yet another embodiments, a sample may be an environmental sample such as, for example, a water sample, soil sample, archeological sample, or other sample collected from a non-living source. In other embodiments, a sample may be a multi-organism sample (e.g., a mixed organism sample). In embodiments, a biological sample is or comprises biological tissue or fluid. In embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; pap smear, oral swabs; nasal swabs; washings or lavages such as ductal lavages or broncheoalveolar lavages; vaginal fluid, uterine lavage fluids, fallopian tube lavage fluids, aspirates; scrapings; bone marrow specimens; tissue biopsy specimens; fetal tissue or fluids; surgical specimens; feces, other body fluids, secretions, and/or excretions; and/or cells therefrom, etc. In embodiments, a biological sample is or comprises cells obtained from an individual. In embodiments, obtained cells are or include cells from an individual from whom the sample is obtained. In embodiments, cells of a biological sample can be sorted or previously sorted or enriched for desired features or characteristics, as is known in the art (e.g., fluorescence activated cell sorting (FACS), antibody pull-down, etc.). In a particular embodiment, a biological sample is a liquid biopsy obtained from a subject. In embodiments, a biological sample is or comprises cells obtained from an individual. In embodiments, obtained cells are or include cells from an individual from whom the sample is obtained. In embodiments, a biological sample can comprise cell-derivatives such as organelles (e.g., nuclei, mitochondria, etc.) or vesicles or exosomes. In embodiments, a sample is a “primary sample” obtained directly from a source of interest by any appropriate means. For example, in embodiments, a primary biological sample is obtained by methods selected from the group consisting of biopsy (e.g., fine needle aspiration or tissue biopsy), surgery, collection of body fluid (e.g., blood, urine, lymph, feces, gynecological fluids, etc.), etc. In embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Cancer: The terms “cancer”, “malignancy”. “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. Cancer is familiar to those experienced in the art as being generally characterized by dysregulated growth of abnormal cells, which may metastasize. Cancers include, by way of non-limiting examples, prostate cancer (e.g, adenocarcinoma, small cell), ovarian cancer (e.g., ovarian adenocarcinoma, serous carcinoma or embryonal carcinoma, yolk sac tumor. teratoma), liver cancer (e.g., HCC or hepatoma, angiosarcoma), plasma cell tumors (e.g., multiple myeloma, plasmacytic leukemia, plasmacytoma, amyloidosis, Waldenstrom's macroglobulinemia), colorectal cancer (e.g., colonic adenocarcinoma, colonic mucinous adenocarcinoma, carcinoid, lymphoma and rectal adenocarcinoma, rectal squamous carcinoma), leukemia (e.g., acute myeloid leukemia, acute lymphocytic leukemia, chronic myeloid leukemia. chronic lymphocytic leukemia, acute myeloblastic leukemia, acute promyelocytic leukemia, acute myelomonocytic leukemia, acute monocytic leukemia, acute erythroleukemia, and chronic leukemia, T-cell leukemia. Sezary syndrome, systemic mastocytosis, hairy cell leukemia, chronic myeloid leukemia blast crisis), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, follicular lymphoma, mantle cell lymphoma. MALT lymphoma, marginal cell lymphoma, Richter's transformation, double hit lymphoma, transplant associated lymphoma, CNS lymphoma, extranodal lymphoma, HIV-associated lymphoma, endemic lymphoma, Burkitt's lymphoma, transplant-associated lymphoproliferative neoplasms, and lymphocytic lymphoma etc.), cervical cancer (e.g., squamous cervical carcinoma, clear cell carcinoma, HPV-associated carcinoma, cervical sarcoma etc.) esophageal cancer (e.g., esophageal squamous cell carcinoma, adenocarcinoma, certain grades of Barretts esophagus, esophageal adenocarcinoma), melanoma (e.g., dermal melanoma, uveal melanoma, acral melanoma, amelanotic melanoma etc.). CNS tumors (e.g., oligodendroglioma, astrocytoma, glioblastoma multiforme, meningioma. schwannoma, craniopharyngioma etc.), pancreatic cancer (e.g., adenocarcinoma, adenosquamous carcinoma, signet ring cell carcinoma, hepatoid carcinoma, colloid carcinoma, islet cell carcinoma, pancreatic neuroendocrine carcinoma etc.), gastrointestinal stromal tumor, sarcoma (e.g., fibrosarcoma, myxosarcoma, liposarcoma, chondrosarcoma, osteogenic sarcoma, angiosarcoma, endothelioma sarcoma, lymphangiosarcoma, lymphangioendothelioma sarcoma, leiomyosarcoma, Ewing's sarcoma, and rhabdomyosarcoma, spindle cell tumor etc.), breast cancer (e.g., inflammatory carcinoma, lobar carcinoma, ductal carcinoma etc.), ER-positive cancer. HER-positive cancer, bladder cancer (squamous bladder cancer, small cell bladder cancer, urothelial cancer etc.), head and neck cancer (e.g., squamous cell carcinoma of the head and neck, HPV-associated squamous cell carcinoma, nasopharyngeal carcinoma etc.), lung cancer (e.g., non-small cell lung carcinoma, large cell carcinoma, bronchogenic carcinoma, squamous cell cancer, small cell lung cancer etc.), metastatic cancer, oral cavity cancer, uterine cancer (leiomyosarcoma, leiomyoma etc.), testicular cancer (e.g., seminoma, non-seminoma, and embryonal carcinoma yolk sack tumor etc.), skin cancer (e.g., squamous cell carcinoma, and basal cell carcinoma, merkel cell carcinoma, melanoma, cutaneous t-cell lymphoma etc.), thyroid cancer (e.g., papillary carcinoma, medullary carcinoma, anaplastic thyroid cancer etc.), stomach cancer, intra-epithelial cancer, bone cancer, biliary tract cancer, eye cancer, larynx cancer, kidney cancer (e.g., renal cell carcinoma, Wilms tumor etc.), gastric cancer, blastoma (e.g., nephroblastoma, medulloblastoma, hemangioblastoma, neuroblastoma, retinoblastoma etc.), myeloproliferative neoplasms (polycythemia vera, essential thrombocytosis, myelofibrosis, etc.), chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, pheochromocytoma, peritoneal mesothelioma, hyperparathyroid adenoma, adrenal cancer, cancer of unknown primary, cancer of the endocrine system, cancer of the penis, cancer of the urethra, cutaneous or intraocular melanoma, a gynecologic tumor, solid tumors of childhood, or neoplasms of the central nervous system, primary mediastinal germ cell tumor, clonal hematopoiesis of indeterminate potential (CHIP), smoldering myeloma, monoclonal gammaglobulinopathy of unknown significant, monoclonal B-cell lymphocytosis, low grade cancers, clonal field defects, preneoplastic neoplasms, ureteral cancer, autoimmune-associated cancers (i.e, ulcerative colitis, primary sclerosing cholangitis, celiac disease), cancers associated with an inherited predisposition (i.e, those carrying genetic defects in such as BRCA/. BRCA2. TP53. PTEN. ATM, etc.) and various genetic syndromes such as MEN1, MEN2 trisomy 21 etc.) and those occurring when exposed to chemicals in utero (i.e, clear cell cancer in female offspring of women exposed to Diethylstilbestrol [DES]), among many others. In embodiments, variants detected, analyzed and/or quantified in the context of the present disclosure are associated with cancer (e.g., neoplastic variants).

Cancer driver or Cancer driver gene: As used herein, “cancer driver” or “cancer driver gene” refers to a genetic lesion that has the potential to allow a cell, in the right context, to undergo, or begin to undergo, a malignant transformation. Such genes include tumor suppressors (e.g., TP53, BRCA1) that normally suppress malignancy transformation and when mutated in certain ways, no longer do. Other driver genes can be oncogenes (e.g., KRAS, EGFR) that when mutated in certain ways become constitutively active or gain new properties that facilitate a cell to become malignant. Other mutations found in non-coding regions of the genome can be cancer drivers. For example, a mutation of the promoter region of the telomerase gene (TERT) can result in overexpression of the gene and thus become a cancer driver. Other mutations in non-coding regions can facilitate aberrant splicing or modulate transcription factor binding or other regulatory changes that can, in certain cases, lead to neoplastic growth. Certain rearrangements (e.g., BCR-ABL fusion) can juxtapose one genetic region with that of another to drive tumorigenesis through mechanisms related to overexpression, loss of repression or chimeric fusion genes. Broadly speaking, genetic mutations (or epimutations) that confer a phenotype to a cell that facilitates its proliferation, survival, or competitive advantage over other cells or that renders its ability to evolve more robust, can be considered a driver mutation. This is to be contrasted with mutations that lack such features, even if they may happen to be in the same gene (i.e., a synonymous mutation). When such mutations are identified in tumors, they are commonly referred to as passenger mutations because they “hitchhiked” along with the clonal expansion without meaningfully contributing to the expansion. As recognized by one of ordinary skill in the art, the distinction of driver and passenger is not absolute and should not be construed as such. Some drivers only function in certain situations (e.g., certain tissues) and others may not operate in the absence of other mutations or epimutations or other factors.

Determine: Many methodologies described herein include a step of “determining” Those of ordinary skill in the art, reading the present specification, will appreciate that such “determining” can utilize or be accomplished through use of any of a variety of techniques available to those skilled in the art, including for example specific techniques explicitly referred to herein. In embodiments, determining involves manipulation of a physical sample. In embodiments, determining involves consideration and/or manipulation of data or information, for example utilizing a computer or other processing unit adapted to perform a relevant analysis. In embodiments, determining involves receiving relevant information and/or materials from a source. In embodiments, determining involves comparing one or more features of a sample or entity to a comparable reference.

Duplex Sequencing (DS): As used herein. “Duplex Sequencing (DS)” is, in its broadest sense, refers to an error-correction method that achieves exceptional accuracy by comparing the sequence from both strands of individual DNA molecules.

Expression: As used herein, “expression” of a nucleic acid sequence refers to one or more of the following events; (1) production of an RNA template from a DNA sequence (e.g., by transcription); (2) processing of an RNA transcript (e.g., by splicing, editing. 5′ cap formation, and/or 3′ end formation); (3) translation of an RNA into a polypeptide or protein; and/or (4) post-translational modification of a polypeptide or protein.

Gene: As used herein, the term “gene” refers to a DNA sequence in a chromosome that codes for a product (e.g., an RNA product and/or a polypeptide product). In embodiments, a gene includes coding sequence (i.e., sequence that encodes a particular product); in embodiments, a gene includes non-coding sequence. In some particular embodiments, a gene may include both coding (e.g., exonic) and non-coding (e.g., intronic) sequences. In embodiments, a gene may include one or more regulatory elements that, for example, may control or impact one or more aspects of gene expression (e.g., cell-type-specific expression, inducible expression, etc.).

Mutation; As used herein, the term “mutation” refers to alterations to nucleic acid sequence or structure relative to a reference sequence. Mutations to a polynucleotide sequence can include point mutations (e.g., single base mutations), multi-nucleotide mutations, nucleotide deletions, sequence rearrangements, nucleotide insertions, and duplications of the DNA sequence in the sample, among complex multi-nucleotide changes. Mutations can occur on both strands of a duplex DNA molecule as complementary base changes (i.e., true mutations), or as a mutation on one strand but not the other strand (i.e., heteroduplex), that has the potential to be either repaired, destroyed or be mis-repaired/converted into a true double-stranded mutation. Reference sequences may be present in databases (i.e., HG38 human reference genome) or the sequence of another sample to which a sequence is being compared. Mutations are also known as genetic variant.

Mutant frequency; As used herein, the term “mutant frequency”, also sometimes referred to as “mutational frequency”, refers to the number of unique mutations detected per the total number of cells interrogated. In embodiments, the mutant frequency may refer to the number of unique mutations detected per the total number of base-pairs sequenced. In embodiments the unique mutations are defined as Duplex Sequencing verified mutations. The total number of cells interrogated may be determined by the number of cell indices identified following data analysis. The total number of base-pairs sequenced may be defined as those verified by Duplex Sequencing. In embodiments, the mutant frequency is the frequency of mutations within only a specific gene, a set of genes, or a set of genomic targets. In embodiments, the mutant frequency is the frequency of cells that comprise a target mutation. In embodiments mutant frequency may refer to only certain types of mutations (for example the frequency of A>T mutations, which is calculated as the number of A>T mutations per the total number of A bases). The frequency at which mutations arise into a population of cells or molecules can vary by age of a subject, over time, by tissue or organization type, by region of a genome, by type of mutation, by trinucleotide context, inherited genetic background, by exposure to mutagenic chemicals, by exposure to radiation, and by exposure to an environment comprising any of the above, among other things.

Nucleic acid: As used herein, in its broadest sense, refers to any compound and/or substance that is or can be incorporated into an oligonucleotide chain. In embodiments, a nucleic acid is a compound and/or substance that is or can be incorporated into an oligonucleotide chain via a phosphodiester linkage. As will be clear from context, in embodiments, “nucleic acid” refers to an individual nucleic acid residue (e.g., a nucleotide and/or nucleoside); in embodiments, “nucleic acid” refers to an oligonucleotide chain comprising individual nucleic acid residues. In embodiments, a “nucleic acid” is or comprises RNA; in embodiments, a “nucleic acid” is or comprises DNA. In embodiments, a nucleic acid is, comprises, or consists of one or more natural nucleic acid residues. In embodiments, a nucleic acid is, comprises, or consists of one or more nucleic acid analogs. In embodiments, a nucleic acid analog differs from a nucleic acid in that it does not utilize a phosphodiester backbone. For example, in embodiments, a nucleic acid is, comprises, or consists of one or more “peptide nucleic acids”, which are known in the art and have peptide bonds instead of phosphodiester bonds in the backbone, are considered within the scope of the present technology. Alternatively, or additionally, in embodiments, a nucleic acid has one or more phosphorothioate and/or 5′-N-phosphoramidite linkages rather than phosphodiester bonds. In embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In embodiments, a nucleic acid is, comprises, or consists of one or more nucleoside analogs (e.g., 2-aminoadenosine, 2-thiothymidine, inosine, pyrrolo-pyrimidine, 3-methyl adenosine. 5-methylcytidine, C-5 propynyl-cytidine, C-5 propynyl-uridine, 2-aminoadenosine, C5-bromouridine, C5-fluorouridine, C5-iodouridine. C5-propynyl-uridine. C5-propynyl-cytidine, C5-methylcytidine, 2-aminoadenosine, 7-deazaadenosine. 7-deazaguanosine, 8-oxoadenosine, 8-oxoguanosine, 0) (6)-methylguanine. 2-thiocytidine, methylated bases, intercalated bases, and combinations thereof). In embodiments, a nucleic acid comprises one or more modified sugars (e.g., 2′-fluororibose, ribose, 2′-deoxyribose, arabinose, and hexose) as compared with those in natural nucleic acids. In embodiments, a nucleic acid has a nucleotide sequence that encodes a functional gene product such as an RNA or protein. In embodiments, a nucleic acid includes one or more introns. In embodiments, nucleic acids are prepared by one or more of isolation from a natural source, enzymatic synthesis by polymerization based on a complementary template (in vivo or in vitro), reproduction in a recombinant cell or system, and chemical synthesis. In embodiments, a nucleic acid is at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In embodiments, a nucleic acid is partly or wholly single stranded; in embodiments, a nucleic acid is partly or wholly double-stranded. In embodiments a nucleic acid has a nucleotide sequence comprising at least one element that encodes, or is the complement of a sequence that encodes, a polypeptide. In embodiments, a nucleic acid has enzymatic activity. In embodiments the nucleic acid serves a mechanical function, for example in a ribonucleoprotein complex or a transfer RNA.

Passenger Mutation; As used herein, the term “passenger mutation” refers to mutations that are identified in a clone of cells but are not believed to have contributed to the clonal expansion itself. This is in contrast to a “driver mutation;” which is reasonably believed to have functionally contributed to clonal expansion itself.

Polynucleotide damage; As used herein, the term “polynucleotide damage” or “nucleic acid damage” refers to damage to a subject's deoxyribonucleic acid (DNA) sequence (“DNA damage”) or ribonucleic acid (RNA) sequence (“RNA damage”) that is directly or indirectly (e.g. a metabolite, or induction of a process that is damaging or mutagenic) caused by or precipitated by ex vivo or in vivo factors (e.g., exposure to a genotoxin, aging, metabolic processes, etc.). Damaged nucleic acid may lead to the onset of a disease or disorder, for example, a disease or disorder associated with genotoxin exposure in a subject, aging, or other mutagenic processes. In embodiments, detection of damaged nucleic acid in a subject may be an indication of a genotoxin exposure. Polynucleotide damage may further comprise chemical and/or physical modification of the DNA in a cell. In embodiments, the damage is or comprises, by way of non-limiting examples, at least one of oxidation, alkylation, deamination, methylation, hydrolysis, hydroxylation, nicking, intra-strand crosslinks, inter-strand cross links, blunt end strand breakage, staggered end double strand breakage, phosphory lation, dephosphorylation, sumoy lation, glycosylation, deglycosylation, putrescinylation, carboxylation, halogenation, formylation, single-stranded gaps, damage from heat, damage from desiccation, damage from UV exposure, damage from gamma radiation damage from X-radiation, damage from ionizing radiation, damage from non-ionizing radiation, damage from heavy particle radiation, damage from nuclear decay, damage from beta-radiation, damage from alpha radiation, damage from neutron radiation, damage from proton radiation, damage from cosmic radiation, damage from high pH, damage from low pH, damage from reactive oxidative species, damage from free radicals, damage from peroxide, damage from hypochlorite, damage from tissue fixation such formalin or formaldehyde, damage from reactive iron, damage from low ionic conditions, damage from high ionic conditions, damage from unbuffered conditions, damage from nucleases, damage from environmental exposure, damage from fire, damage from mechanical stress, damage from enzymatic degradation, damage from microorganisms, damage from preparative mechanical shearing, damage from preparative enzymatic fragmentation, damage having naturally occurred in vivo, damage having occurred during fixation, damage having occurred during nuclei extraction, damage having occurred during permeabilization, damage having occurred during nucleic acid extraction, damage having occurred during sequencing library preparation, damage having been introduced by a polymerase, damage having been introduced during nucleic acid repair, damage having occurred during nucleic acid end-tailing, damage having occurred during nucleic acid ligation, damage having occurred during sequencing, damage having occurred from mechanical handling of DNA, damage having occurred during passage through a nanopore, damage having occurred as part of aging in an organism, damage having occurred as a result if chemical exposure of an individual, damage having occurred by a mutagen, damage having occurred by a carcinogen, damage having occurred by a clastogen, damage having occurred from in vivo inflammation damage due to oxygen exposure, damage due to one or more strand breaks, and any combination thereof.

Reference; As used herein describes a standard or control relative to which a comparison is performed. For example, in embodiments, an agent, animal, individual, population, sample, sequence or value of interest is compared with a reference or control agent, animal, individual, population, sample, sequence or value. In embodiments, a reference or control is tested and/or determined substantially simultaneously with the testing or determination of interest. In embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Single Nucleotide Polymorphism (SNP); As used herein, the term “single nucleotide polymorphism” or “SNP” refers to a particular base position in the genome where alternative bases are known to distinguish one allele from another. SNPs refer to variations that are single nucleotide in nature as opposed to MNVs which refer to multinucleotide variants. A “copy number polymorphism” or “copy number variant” (referred to as CNPs or CNVs) refers to a variation in the number of copies of a sequence within the DNA. In embodiments, one or a few SNPs and/or CNPs is/are sufficient to distinguish complex genetic variants from one another so that, for analytical purposes, one or a set of SNPs and/or CNPs may be considered to be characteristic of a particular variant, trait, cell type, individual, species, etc., or set thereof. In embodiments, one or a set of SNPs and/or CNPs may be considered to define a particular variant, trait, cell type, individual, species, etc., or set thereof. In the most common usage, SNP generally implies the variant in question is inherited from the germline. The broader term Single Nucleotide Variant (SNV) may entail an inherited SNP or a somatically acquired mutation.

Strand Defining Element (SDE); As used herein, the term “Strand Defining Element” or “SDE”, refers to any material which allows for the identification of a specific strand of a double-stranded nucleic acid material and thus differentiation from the other/complementary strand (e.g., any material that renders the amplification products of each of the two single stranded nucleic acids resulting from a target double-stranded nucleic acid substantially distinguishable from each other after sequencing or other nucleic acid interrogation). In embodiments, an SDE may be or comprise one or more segments of substantially non-complementary sequence within an adapter sequence. In particular embodiments, a segment of substantially non-complementary sequence within an adapter sequence can be provided by an adapter molecule comprising a Y-shape or a “loop” shape. In other embodiments, a segment of substantially non-complementary sequence within an adapter sequence may form an unpaired “bubble” in the middle of adjacent complementary sequences within an adapter sequence. In other embodiments an SDE may encompass a nucleic acid modification. In embodiments an SDE may comprise physical separation of paired strands into physically separated reaction compartments. In embodiments an SDE may comprise a chemical modification. In embodiments an SDE may comprise a modified nucleic acid. In embodiments an SDE may relate to a sequence variation in a nucleic acid molecule caused by random or semi-random damage, chemical modification, enzymatic modification or other modification to the nucleic acid molecule. In embodiments the modification may be deamination of methylcytosine. In embodiments the modification may entail sites of nucleic acid nicks. Various embodiments of SDEs are further disclosed in International Patent Publication Nos. WO2013/142389, WO2017/100441, and WO2018/175997, all of which are incorporated by reference herein in their entireties.

Subject; As used herein, the term “subject” refers to an organism, typically a mammal (e.g., a human, in embodiments including prenatal human forms). In embodiments, a subject is suffering from a relevant disease, disorder or condition. In embodiments, a subject is susceptible to a disease, disorder, or condition. In embodiments, a subject displays one or more symptoms or characteristics of a disease, disorder or condition. In embodiments, a subject does not display any symptom or characteristic of a disease, disorder, or condition. In embodiments, a subject is someone with one or more features characteristic of susceptibility to or risk of a disease, disorder, or condition. In embodiments, a subject is a patient. In embodiments, a subject is an individual to whom diagnosis and/or therapy is and/or has been administered.

Substantially; As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Treatment; As used herein, the term “treatment” refers to the application or administration of a therapeutic agent to a subject, or application or administration of a therapeutic agent to an isolated tissue or cell line from a subject, who has a disorder, e.g., a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. Treatment may also refer to the application of an exposure or process to a subject or cells for the purpose of inducing a change that is not intended to cure, heal, alleviate, relieve, alter, remedy, ameliorate, improve, or affect the disease, the symptoms of disease. For example, a laboratory animal may be treated with a harmful chemical, for the purpose of assessing its adverse effect on the rodent subject, such as to predict its effect on humans.

Unique Molecular Identifier (UMI); As used herein, the term “unique molecular identifier” or “UMI”, (which may be referred to as a molecular “tag” a molecule “barcode”, a “molecular barcode”, a “Single Molecular Identifier”, or “SMI”, among other names) refers to any material (e.g., a nucleotide sequence, a nucleic acid molecule feature) that is capable of distinguishing an individual molecule in a large heterogeneous population of molecules. In embodiments, a UMI can be or comprise an exogenously applied UMI. In embodiments, an exogenously applied UMI may be or comprise a degenerate or semi-degenerate sequence. As used herein, a “degenerate sequence” is a nucleotide sequence that is known or unknown in which every nucleotide position is unrestricted in its nucleotide variability. Also as used herein, a “semi-degenerate sequence” is a nucleotide sequence that is known or unknown in which at least one. but not all, nucleotide positions are fixed or restricted in their nucleotide variability. In embodiments, a UMI may be randomly generated or synthesized, semi-randomly generated or synthesized, or can be non-randomly generated or synthesized (e.g., defined). In embodiments, a UMI may comprise a known nucleic acid sequence from within a pool of known sequences. In embodiments, a UMI can be or comprise an endogenous UMI. In embodiments, an endogenous UMI may be or comprise information related to specific fragment ends (e.g., shear points, mapping start or stop points relative to a reference sequence, etc.), or features relating to the terminal ends of individual molecules of or comprising a target sequence. In embodiments, a UMI may relate to a sequence variation in a nucleic acid molecule caused by random or semi-random damage, chemical modification, enzymatic modification or other modification to the nucleic acid molecule. In embodiments, the modification may be deamination of methylcytosine. In embodiments the modification may entail sites of nucleic acid nicks. In embodiments, a UMI may comprise both exogenous and endogenous elements. In embodiments, a UMI may comprise physically adjacent UMI elements. In embodiments. UMI elements may be spatially distinct in a molecule. In embodiments a UMI may be a non-nucleic acid. In embodiments, a UMI may comprise two or more different types of UMI information. Various embodiments of UMIs are further disclosed in International Patent Publication Nos. WO2013/142389, WO2017/100441, and WO2018/175997, all of which are incorporated by reference herein in their entireties.

Variant; As used herein, the term “variant” refers to an entity that shows significant structural identity with a reference entity, but differs structurally from the reference entity in the presence or level of one or more chemical moieties as compared with the reference entity. In the context of nucleic acids, a variant nucleic acid may have a characteristic sequence element comprised of a plurality of nucleotide residues having designated positions relative to another nucleic acid in linear or three-dimensional space. Sequences with homology differ by one or more variant. For example, a variant polynucleotide (e.g., DNA) may differ from a reference polynucleotide as a result of one or more differences in nucleic acid sequence. In embodiments, a variant polynucleotide sequence includes an insertion, deletion, substitution or mutation relative to another sequence (e.g., a reference sequence or other polynucleotide (e.g., DNA) sequences in a sample). Examples of variants include SNPs, SNVs, CNVs, CNPs, MNVs, MNPs, mutations, cancer mutations, driver mutations, passenger mutations, inherited polymorphisms.

Variant frequency; As used herein, the term “variant frequency” refers to the relative frequency of a genetic variant at a particular locus in a population, expressed as a fraction or percentage of the population. The population may be a population of cells, a population of organisms, a population of subjects, or a population of molecules or a population of DNA molecules, among others.

Variant allele frequency; As used herein, the term “variant allele frequency” refers to the relative frequency of an allele (variant of a gene) at a particular locus in a population (e.g., a fraction of all chromosomes in the population that carry a particular allele among a population of cells, a population of organisms, a population of subjects, or a population of molecules or a population of DNA molecules, among others.

### Selected Embodiments of Single-Cell Duplex Sequencing Methods and Associated Adapters and Reagents

Duplex Sequencing (DS) is a method for producing error-corrected DNA sequences from double stranded nucleic acid molecules, and which was originally described in International Patent Publication Nos. WO 2013/142389. WO 2017/100441, WO2018/175997, and in U.S. Pat. No. 9,752,188, in Schmitt et al., (Detection of ultra-rare mutations by next-generation sequencing. PNAS. 2012; 109 (36); 14508-14513); in Kennedy et, al. (Ultra-Sensitive Sequencing Reveals an Age-Related Increase in Somatic Mitochondrial Mutations that are inconsistent with oxidative damage. PLOS Genetics. 2013; 9 (9); 1-10); in Kennedy et al . . . (Detecting ultralow-frequency mutations by Duplex Sequencing. Nat Protoc. 2014; 9 (11); 2586-2606); and in Schmitt et al., (Sequencing small genomic targets with high efficiency and extreme accuracy. Nature Methods. 2015; 12 (5); 423-5). Each of the above-mentioned patents, patent applications and publications are incorporated herein by reference in their entireties. With reference to FIG. 1 and in embodiments, DS can be used to independently sequence both strands of individual DNA molecules in such a way that the derivative sequence reads can be recognized as having originated from the same double-stranded nucleic acid parent molecule during massively parallel sequencing (MPS), also commonly known as next generation sequencing (NGS), but also differentiated from each other as distinguishable entities following sequencing. The resulting sequence reads from each strand are then compared for the purpose of obtaining an error-corrected sequence of the original double-stranded nucleic acid molecule known as a Duplex Consensus Sequence (DCS). The process of DS makes it possible to explicitly confirm that both strands of an original double-stranded nucleic acid molecule are represented in the generated sequencing data used to form a DCS (e.g., FIG. 1, 100).

After providing double-stranded nucleic acid molecules from a biological source (e.g., FIG. 1, 102), various method steps can be performed to produce nucleic acid sequencing libraries suitable for generating duplex error-corrected sequences. In certain embodiments described herein, methods incorporating DS may include ligation or tagmentation of one or more adapters (e.g., barcoded adapters, sequencing adapters, etc.) to double-stranded nucleic acid molecules. Resultant double-stranded nucleic acid molecule complexes comprise a first strand target nucleic acid sequence and a second strand target nucleic sequence (e.g., FIG. 1. 104).

In various embodiments, a resulting nucleic acid complex can include at least one UMI sequence. which may entail an exogenously applied degenerate or semi-degenerate sequence, endogenous information related to the specific fragment ends (e.g., shear-points) or fragment end-segments of the double-stranded nucleic acid molecule, or a combination thereof. The UMI can render the nucleic acid molecule substantially distinguishable from the plurality of other molecules in a population being sequenced either alone or in combination with distinguishing elements of the nucleic acid fragments to which they were incorporated. The UMI element's substantially distinguishable feature can be independently carried by each of the single strands that form the double-stranded nucleic acid molecule such that derivative amplification products of each strand can be recognized as having come from the same original double-stranded nucleic acid molecule after sequencing. In other embodiments, the UMI may include additional information and/or may be used in other methods for which such molecule distinguishing functionality is useful, such as those described in the above-referenced publications. In another embodiment, the UMI element may be incorporated after adapter incorporation. In embodiments, the UMI is incorporated as a double-stranded element. In other embodiments, the UMI is initially incorporated as a single-stranded element (e.g., the UMI can be on a single-stranded portion(s) of the adapter). In certain embodiments, the initially single-stranded UMI element can be copied (e.g., as complementary sequence by polymerase extension, gap-filling followed by nick repair, etc.) to form a double-stranded UMI element. In other embodiments, the UMI comprises a combination of single-stranded and double-stranded elements.

In embodiments, each double-stranded nucleic acid sequence complex can further include an element (e.g., an SDE) that renders the amplification products of the two single-stranded nucleic acids that form the double-stranded nucleic acid molecule substantially distinguishable from each other after sequencing. In embodiments, an SDE may comprise asymmetric primer sites (e.g., for hybridization to amplification primers or sequencing primers) comprised within the incorporated adapters, or, in other arrangements, sequence asymmetries may be introduced into the adapter molecules not within the primer sequences, such that at least one position in the nucleotide sequences of the first strand target nucleic acid sequence complex and the second stand of the target nucleic acid sequence complex are different from each other following amplification and sequencing. In other embodiments, the UMI may comprise another biochemical asymmetry between the two strands that differs from the canonical nucleotide sequences A. T. C, G or U, but is converted into at least one canonical nucleotide sequence difference in the two amplified and sequenced molecules. In yet another embodiment, the SDE may be a means of physically separating the two strands before amplification, such that the derivative amplification products from the first strand nucleic acid sequence and the second strand nucleic acid sequence are maintained in substantial physical isolation from one another for the purposes of maintaining a distinction between the two. Other such arrangements or methodologies for providing an SDE function that allows for distinguishing the first and second strands may be utilized, such as those described in the above-referenced publications, or other methods that serves the functional purpose described.

After generating the population of double-stranded nucleic acid complexes wherein any particular complex comprises at least one UMI and, optionally, at least one SDE, or where one or both of these elements will be subsequently introduced, the complex can be subjected to DNA amplification, such as with PCR, or any other biochemical method of DNA amplification (e.g., rolling circle amplification, multiple displacement amplification, isothermal amplification, bridge amplification or surface-bound amplification, such that one or more copies of the first strand nucleic acid sequence and one or more copies of the second strand nucleic acid sequence are produced (e.g., FIG. 1, 106). The one or more amplification copies of the first strand nucleic acid molecule and the one or more amplification copies of the second strand nucleic acid molecule can then be subjected to DNA sequencing, preferably using a “Next-Generation” massively parallel DNA sequencing platform (e.g., FIG. 1, 108).

The sequence reads produced from either the first strand nucleic acid molecule and the second strand nucleic acid molecule, derived from the original double-stranded nucleic acid molecule, can be identified based on sharing a related UMI and distinguished from the opposite strand nucleic acid molecule by virtue of an SDE. In embodiments, the UMI may be a sequence based on a mathematically-based error correction code (for example, a Hamming code), whereby certain amplification errors, sequencing errors or UMI synthesis errors can be tolerated for the purpose of relating the sequences of the UMI sequences on complementary strands of an original duplex (e.g., a double-stranded nucleic acid molecule). For example, with a double-stranded exogenous UMI where the UMI comprises 15 base pairs of fully degenerate sequence of canonical DNA bases, a possible 415=1.073,741,824 individual UMIs will exist in such a population. In a particular example, if two UMIs are recovered from reads of sequencing data that differ by only one nucleotide within the UMI sequence out of a population of 10,000 sampled UMIs, one can mathematically calculate the probability of this occurring by random chance, a decision can be made whether it is more probable that the single base pair difference reflects one of the aforementioned types of errors, and the UMI sequences could be determined to have in fact derived from the same original duplex molecule. In embodiments where the UMI is, at least in part, an exogenously applied sequence where the sequence variants are not fully degenerate to each other and are, at least in part, known sequences, the identity of the known sequences can in embodiments be designed in such a way that one or more errors of the aforementioned types will not convert the identity of one known UMI sequence to that of another UMI sequence, such that the probability of one UMI being misinterpreted as that of another UMI is reduced. In embodiments, this UMI design strategy comprises a Hamming Code approach or derivative thereof.

Once identified, one or more sequence reads produced from the first strand nucleic acid molecule are compared with one or more sequence reads produced from the corresponding second strand nucleic acid molecule (e.g., FIG. 1. 110). In embodiments, technical errors represented in the compared sequencing reads (e.g., in the first and/or second sequence reads) may be identified as nucleotide positions where the first and second strand sequence reads disagree (e.g., FIG. 1. 112). In embodiments, DS includes producing an error-corrected nucleic acid molecule sequence. For example, nucleotide positions where the bases from both the first and second strand target nucleic acid sequences agree are deemed to be true sequences, whereas nucleotide positions that disagree between the two strands are recognized as potential sites of technical errors that may be discounted, eliminated, corrected or otherwise identified. An error-corrected sequence of the original double-stranded target nucleic acid molecule can thus be produced. In embodiments and following separately grouping of each of the sequencing reads produced from the first strand target nucleic acid molecule and the second strand target nucleic acid molecule, a single-strand consensus sequence can be generated for each of the first and second strands. The single-stranded consensus sequences from the first strand target nucleic acid molecule and the second strand target nucleic acid molecule can then be compared to produce an error-corrected target nucleic acid molecule sequence.

Alternatively, in embodiments, sites of sequence disagreement between the two strands can be recognized as potential sites of biologically-derived mismatches in the original double-stranded nucleic acid molecule. Alternatively, in embodiments, sites of sequence disagreement between the two strands can be recognized as potential sites of DNA synthesis-derived mismatches in the original double-stranded nucleic acid molecule. Alternatively, in embodiments, sites of sequence disagreement between the two strands can be recognized as potential sites where a damaged or modified nucleotide base was present on one or both strands and was converted to a mismatch by an enzymatic process (for example a DNA polymerase, a DNA glycosylase or another nucleic acid modifying enzyme or chemical process). In embodiments, this latter finding can be used to infer the presence of nucleic acid damage or nucleotide modification prior to the enzymatic process or chemical treatment.

In embodiments, sequencing reads generated from the DS steps discussed herein can be further filtered to eliminate sequencing reads from DNA-damaged molecules (e.g., damaged during storage, shipping, during or following tissue or blood extraction, during or following library preparation, etc.). For example, DNA repair enzymes, such as Uracil-DNA Glycosylase (UDG). Formamidopyrimidine DNA glycosylase (FPG), and 8-oxoguanine DNA glycosylase (OGGI), can be utilized to eliminate or correct DNA damage (e.g., in vitro DNA damage or in vivo damage). These DNA repair enzymes, for example, are glycoslyases that remove damaged bases from DNA. For example, UDG removes uracil that results from cytosine deamination (caused by spontaneous hydrolysis of cytosine) and FPG removes 8-oxo-guanine (e.g., a common DNA lesion that results from reactive oxygen species). FPG also has lyase activity that can generate a I base gap at abasic sites. Such abasic sites will generally subsequently fail to amplify by PCR, for example, because the polymerase fails to copy the template. Accordingly, the use of such DNA damage repair/elimination enzymes can effectively remove damaged DNA that doesn't have a true mutation but might otherwise be undetected as an error following sequencing and duplex sequence analysis. Although an error due to a damaged base can often be corrected by DS, in rare cases a complementary error could theoretically occur at the same position on both strands; thus, reducing error-increasing damage can reduce the probability of artifacts. Furthermore, during library preparation, certain fragments of DNA to be sequenced may be single-stranded from their source or from processing steps. These regions are typically converted to double-stranded DNA during an “end repair” step known in the art, whereby a DNA polymerase and nucleotide substrates are added to the cell samples to extend 5′ recessed ends of the target DNA. A mutagenic site of DNA damage in the single-stranded portion of the DNA being copied (i.e, single-stranded 5′ overhang at one or both ends of the DNA duplex or internal single-stranded nicks or gaps) can cause an error during the fill-in reaction that could render a single-stranded mutation, synthesis error, or site of nucleic acid damage into a double-stranded form that could be misinterpreted in the final duplex consensus sequence as a true mutation. This scenario, termed “pseudo-duplex”, can be reduced or prevented by use of such damage destroying/repair enzymes. In other embodiments, this occurrence can be reduced or eliminated through use of strategies to destroy or prevent single-stranded portions of the original duplex molecule to form (e.g, use of certain enzymes being used to fragment the original double stranded nucleic acid material rather than other enzymes that may leave nicks or gaps). In other embodiments, use of processes to eliminate single-stranded portions of original double-stranded nucleic acids (e.g, single-stand specific nucleases such as SI nuclease or mung bean nuclease) can be utilized for a similar purpose.

In further embodiments, sequencing reads generated from the DS steps discussed herein can be further filtered to eliminate the false mutations associated with the “pseudo-duplex” molecules discussed above by trimming ends of the reads most prone to pseudo-duplex artifacts during data analysis. As these artifacts of library preparation can incorrectly appear to be true mutations once sequenced, certain steps can be taken during post-sequencing analysis including computationally trimming the ends of the sequencing reads to exclude any mutations that may have occurred in higher risk regions, thereby reducing the number of false mutations. In embodiments, such trimming of sequencing reads can be accomplished automatically (e.g., a normal process step), in another embodiment, a mutation frequency can be assessed for fragment end regions and if a threshold level of mutations is observed in the fragment end regions, sequencing read trimming can be performed before generating a double-strand consensus sequence read of the DNA fragments.

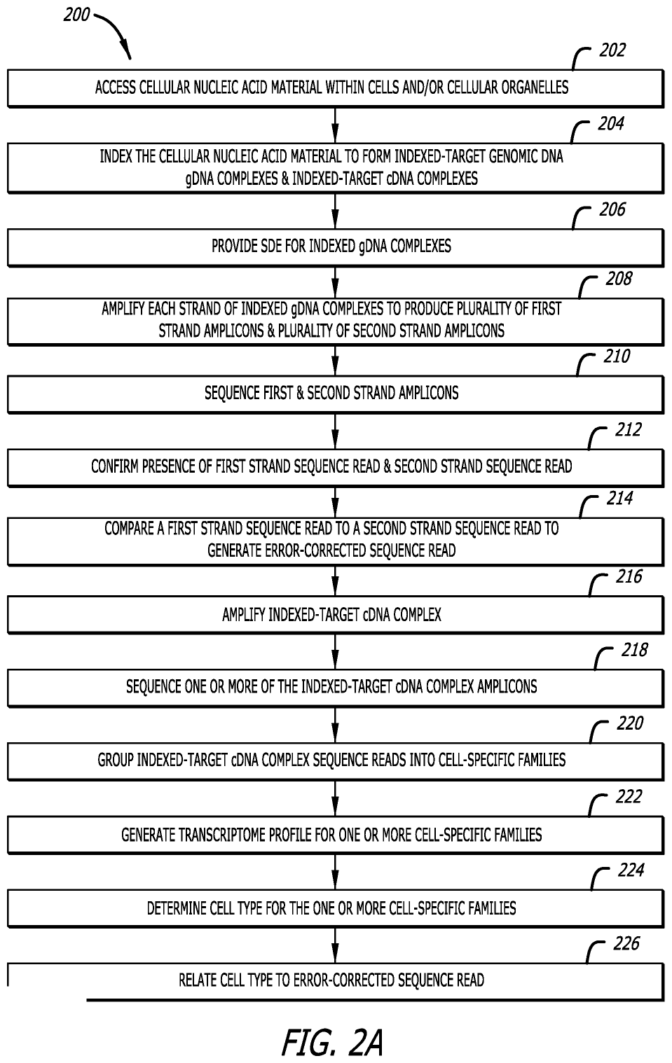

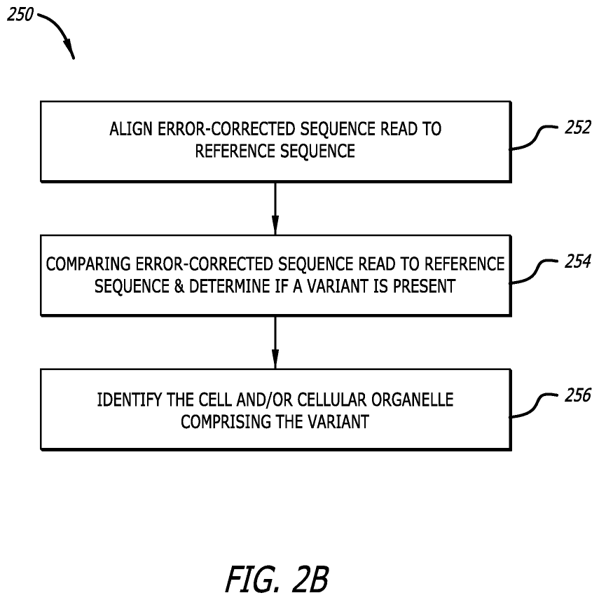

Aspects of the present disclosure are directed to methods of single-cell DS. In certain embodiments, the sequencing accuracy and sensitivity of variant detection achieved by DS method steps provided in a cell-specific or cell origin identifiable manner (e.g., FIG. 2A, 200). By way of specific example illustrated in the flow diagram of FIG. 2A, and in embodiments, provided herein are methods of generating an error-corrected sequence read of a double-stranded genomic DNA (gDNA) material in a cell-specific or cell-identifiable manner. Such methods include steps of accessing cellular nucleic acid material within cells and/or cellular organelles, wherein the cellular nucleic acid material comprises the double-stranded gDNA material and double-stranded cDNA material derived from RNA within the cells and/or cellular organelles (e.g., . FIG. 2A. 202). The method also includes indexing the cellular nucleic acid material thereby forming indexed-target nucleic acid complexes having a first population of complexes comprising indexed-target gDNA complexes and a second population of complexes comprising indexed-target cDNA complexes, wherein each indexed-target nucleic acid complex in a plurality of the indexed-target nucleic acid complexes comprises (a) a cell index that identifies the target nucleic acid material as originating from a particular cell among a population of cells, and (b) a molecular index comprising a UMI sequence that identifies the indexed-target nucleic acid complex among the first and second populations of complexes (e.g., FIG. 2A, 204). The method may also include providing an SDE for the indexed-target nucleic acid complexes of the first population, wherein the SDE identifies a particular strand of a particular indexed-target nucleic acid complex (e.g., . FIG. 2A, 206). For one or more indexed-target nucleic acid complexes in the first population, the method can include the steps of amplifying each strand of the indexed-target nucleic acid complex to produce a plurality of first strand amplicons and a plurality of second strand amplicons (e.g., FIG. 2A, 208). The method may also include the steps of sequencing one or more of the first strand amplicons and one or more of the second strand amplicons to produce one or more first strand sequence reads and one or more second strand sequence reads, and confirming the presence of at least one first strand sequence read and at least one second strand sequence read (e.g., FIG. 2A, and 212). The method may further include comparing the at least one first strand sequence read with the at least one second strand sequence read and generating an error-corrected sequence read of the double-stranded target nucleic acid material (e.g., FIG. 2A, 214) by discounting nucleotide positions that do not agree, or alternatively removing compared first and second strand sequence reads having one or more nucleotide positions where the compared first and second strand sequence reads are non-complementary.

For one or more indexed-target nucleic acid complexes in the second population, the method may also include amplifying the indexed-target cDNA complex to produce a plurality of indexed-target cDNA complex amplicons (e.g., FIG. 2A, 216), and sequencing one or more of the indexed-target cDNA complex amplicons to produce one or more indexed-target cDNA complex sequence reads (e.g., FIG. 2A, 218). The method may also include grouping the indexed-target cDNA complex sequence reads into a cell-specific family using the cell index (e.g., FIG. 2A. 220). In embodiments, the method further comprises generating a transcriptome profile for one or more cell-specific families and determining a cell type for the one or more cell-specific families based on the transcriptome profile (e.g., FIG. 2A. 222, 224). In embodiments, the method also comprises determining the cell type associated with the error-corrected sequence read of the double-stranded target nucleic acid material (e.g., FIG. 2A. 226). In other embodiments, the method includes identifying the cell and/or cellular organelle from which the error-corrected sequence read of the double-stranded target nucleic acid material is derived.

In embodiments, cDNA molecules having molecular indices can be subjected to molecule counting, as is known in the art. For example, a UMI associated with a cDNA molecule can be used to deduplicate or identify redundancy of sequence reads derived from the same cDNA molecule, such that each original RNA molecule can be counted one time. Molecular counting can be considered to be part of a cell or transcriptome profile, in accordance with aspects of the present technology.

In embodiments and with reference to FIG. 2B, the method (250) further includes aligning the error-corrected sequence read of the double-stranded target nucleic acid material to a reference sequence (e.g., FIG. 2B, 252) and determining if there is a variant present in the double-stranded target nucleic acid material by comparing the error-corrected sequence read to the reference sequence (e.g., FIG. 2B, 254). The method may also include identifying the cell and/or cellular organelle comprising the variant (e.g., . FIG. 2B. 256). In another embodiment, the step of determining if there is a variant present can include determining the presence of a plurality of variants present in the double-stranded target nucleic acid material originating from a particular cell.

By way of an additional specific example, in embodiments, provided herein are methods of identifying a DNA variant in a cell within a population of cells including the steps of providing a population of cells from a biological sample and accessing cellular nucleic acid material within cells of the population, wherein the cellular nucleic acid material comprises double-stranded gDNA material and single-stranded RNA material within the cells. The method can further include indexing the double-stranded gDNA material to generate indexed-target gDNA complexes, wherein each indexed-target gDNA complex in a plurality of the indexed-target gDNA complexes comprises (a) a cell index that identifies the target gDNA material as originating from a particular cell among the population of cells, and (b) a UMI that identifies the indexed-target gDNA complex among the plurality of the indexed-target gDNA complexes. The method can also include the step of providing an SDE for the indexed-target gDNA complexes, wherein the SDE identifies a particular strand of a particular indexed-target gDNA complex. The method can further include the steps of reverse transcribing the single-stranded RNA material within the cells to generate double stranded cDNA material and indexing the double-stranded cDNA material to generate indexed-target cDNA complexes, wherein each indexed-target cDNA complex in a plurality of the indexed-target cDNA complexes comprises (a) the cell index that identifies the target gDNA material originating from the same particular cell among the population of cells, and (b) a UMI that identifies the indexed-target cDNA complex among the plurality of the indexed-target cDNA complexes and indexed-target gDNA complexes. In embodiments, for one or more indexed-target gDNA complexes in the plurality of the indexed-target gDNA complexes, the method further includes the steps of amplifying each of a first strand and a second strand of the indexed-target gDNA complex, resulting in each strand generating a distinct. yet related, set of amplified indexed-target gDNA products, sequencing each of a plurality of first strand indexed-target gDNA products and a plurality of second strand indexed-target nucleic acid products, and confirming the presence of at least one sequence read from each strand of the indexed-target gDNA complex. The method may also include comparing the at least one sequence read obtained from the first strand with the at least one sequence read obtained from the second strand to form a consensus sequence read of the indexed-target gDNA complex having only nucleotide bases at which the sequence of both strands of the indexed-target gDNA complex are in agreement, such that a variant occurring at a particular position in the consensus sequence read is identified as a true DNA variant. In embodiments, the step of confirming the presence of at least one sequence read from each strand comprises identifying the presence of a first strand sequence read and a second strand sequence read using the cell index, the UMI and the SDE.

In embodiments, and wherein for one or more indexed-target cDNA complexes in the plurality of the indexed-target cDNA complexes, the method may also include amplifying the indexed-target cDNA complex to produce a set of amplified indexed-target cDNA products. sequencing one or more of the amplified indexed-target cDNA products to generate one or more indexed-target cDNA complex sequence reads, and grouping the indexed-target cDNA complex sequence reads into a cell-specific family using the cell index. In embodiments, the step of grouping the indexed-target cDNA complex sequence reads comprises grouping indexed-target cDNA complex sequence reads that share the same cell-index. In other embodiments, the step of grouping the indexed-target cDNA complex sequence reads comprises grouping indexed-target cDNA complex sequence reads that share the same cell-index and UMI. In further embodiments, the method also includes generating a transcriptome profile for one or more cell-specific families and determining a cell type for the one or more cell-specific families based on the transcriptome profile. In embodiments, the method further includes determining the cell type associated with the consensus sequence read of the double-stranded gDNA material. In embodiments, the method includes identifying the cell within the population of cells from which the consensus sequence read of the double-stranded gDNA material is derived.

One of ordinary skill in the art will recognize that DS method steps and reagents, workflow schemes, and protocols described herein may be used with various single-cell DS steps and reagents described herein.

In embodiments, provided herein are methods of generating a high accuracy consensus sequence from a double-stranded nucleic acid material in a cell-specific or cell-identifiable manner. In further embodiments, provided herein are methods of detecting and/or quantifying DNA mutations and/or variants from a sample in a cell-specific or cell-identifiable manner.

The high degree of error correction provided by the strand-comparison technology of Duplex Sequencing reduces sequencing errors of double-stranded nucleic acid molecules by multiple orders of magnitude as compared with standard next-generation sequencing methods. In particular embodiments, single-cell DS can also be used for the accurate detection of sequence variants in one or more cells among a population of normal or non-variant containing cells. One non-limiting example of this application is detection of a small number of abnormal or mutant-containing cells among a larger number of healthy cells (e.g., comprising non-mutated DNA). Such detection can be used in embodiments for determining the presence or absence of a disease state within a subject. In embodiments, the disease state can be cancer. In other embodiments, the disease state can be an elevated risk of developing cancer. In particular embodiments, methods described herein can be used to identify disease-associated mutations harbored within particular cells. In embodiments, methods described herein can be used to identify combinations of variants (e.g., mutations, disease-associated mutations) harbored within particular embodiments.

### Selected Embodiments for the Preparation of Sequencing Libraries for use in Single-Cell Duplex Sequencing Methods and Associated Adapters and Reagents

In general, aspects of the present technology provide approaches for detection of low frequency biological variants in a high throughput manner (i.e., thousands of cells at a time). The approaches utilize cell indexing for unique labeling of cellular nucleic acid molecules within cells and/or cellular organelles (e.g., nuclei, mitochondria), and strand indexing for unique labeling of each strand of (double-stranded) cellular nucleic acid molecules. The approaches enable differentiation of technical variants that occur during NGS workflows from biological variants and real mutations in a cell-specific or cell identifiable manner. In certain embodiments, the methods described herein provide for the assignment of identified variants or combinations of variants to particular cells (e.g., cell types, cells from particular biological samples, etc.) within a population of cells (e.g., a heterogenous mixture of cells/cell types, a homogenous mixture of cells/cell types). Accordingly, the present technology provides for the sensitive detection of variants provided by DS with the specificity of single-cell interrogation in a high throughput manner.

As described herein, the present technology provides a method for preparing a sequencing library for duplex sequencing (DS) detection of low-frequency variants in cells. The method comprises introducing a sample index to cellular nucleic acid molecules of cells of a biological sample, introducing a cell index to the cells according to a method of cell indexing, introducing a molecular index as a UMI to the cellular nucleic acid molecules, and introducing a strand index as an SDE to the cellular nucleic acid molecules.

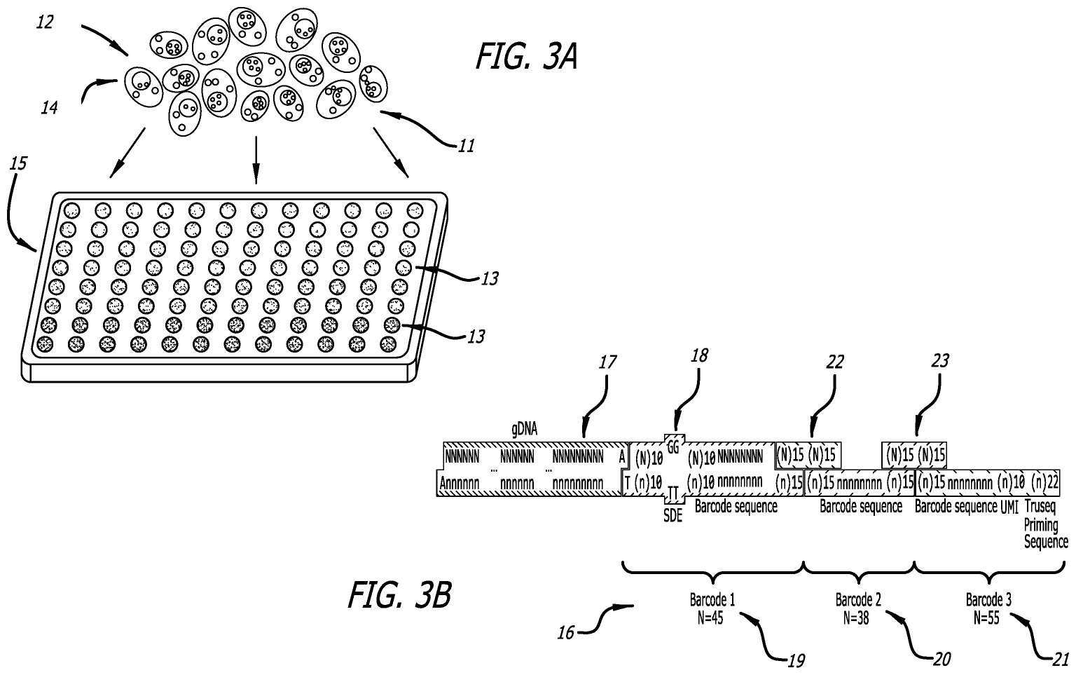

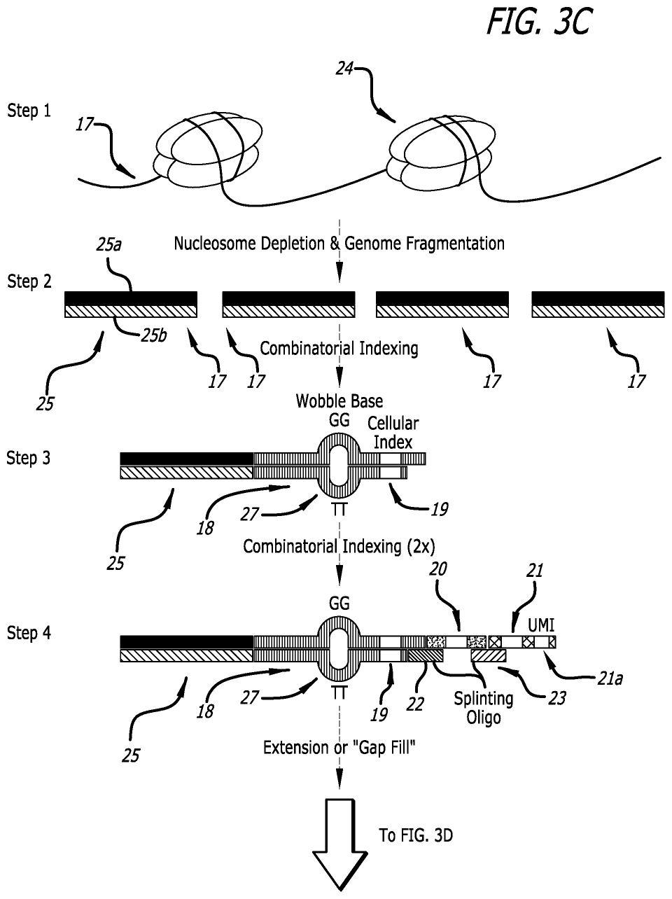

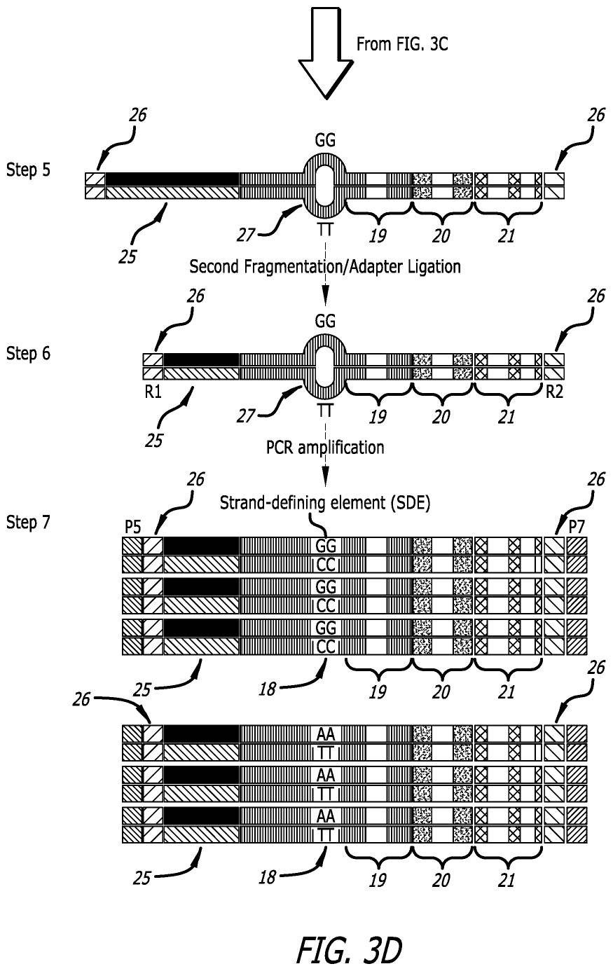

In embodiments and as part of sequence data analysis, the sample index is for identification of a particular biological sample among biological samples thereby enabling multiplexing of samples, the cell index is for identification of a particular cell among a population of cells, the molecular index is for identification of a particular cellular nucleic acid molecule among cellular nucleic acid molecules, and the strand index is for identification of a particular strand of the particular cellular nucleic acid molecule. In this manner, biological nucleic acid variants can be detected within samples and can be effectively correlated with particular cells or cell types, for example, as can be determined with transcriptome or other analyses. While any method of cell indexing can be implemented certain embodiments are described in detail herein and with respect to FIGS. 3A-9B.

In embodiments, methods are provided for generating sequencing libraries for single-cell DS. FIG. 3A is a conceptual illustration of various single-cell DS methods steps in accordance with embodiments of the present technology. For example. FIG. 3A illustrates a high-level overview of a combinatorial approach of cell indexing. whereby nucleic acids such as genomic DNA (gDNA) within a plurality of cells are labeled according to a cell index (e.g., FIG. 3B). While the method is shown for gDNA, the same or a similar method can be performed with other cellular nucleic acid molecules, including but not necessarily limited to mitochondrial DNA (mtDNA), RNA, and the like. In instances where RNA is to be prepared for sequencing, a reverse transcription step can be performed to produce complementary DNA (cDNA), as is known in the art.

With reference to FIGS. 3A and 3B together, a plurality of cells 11, initially without any cell index or other labelling, is subjected to an iterative barcode labeling procedure for cell indexing whereby cellular nucleic acid material therein are given unique (or substantially unique) labels or codes such that any particular sequence generated from the resultant sequencing library may be grouped with other information derived from any same particular individual cell 12. Referring to FIGS. 3A and 3B together, and at the conclusion of the procedure, nucleic acid molecules (e.g., 17) within individual cells 12 are uniquely (or almost uniquely, e.g., about 100%, about 99%, about 98%, about 97%, about 96%, about 95%, about 90%, about 85%, about 80%, about 75%, about 70%, about 65%, about 60%, about 55%, or about 50% uniquely) labeled or tagged with barcode combinations resulting in a cell index 16 that is unique (or substantially unique, e.g., about 100%, about 99%, about 98%, about 97%, about 96%, about 95%, about 90%, about 85%, about 80%, about 75%, about 70%, about 65%, about 60%, about 55%, or about 50% unique) to the nucleic acid molecules within individual cells 12. In the illustrated embodiment of FIG. 3A, the plurality of cells 11 are initially pooled to form pooled cells 14, then split into a plurality of aliquots 13 as split cells 15. Once split or divided, the cells of each aliquot are interrogated and steps are provided for generating modified nucleic acid molecules (e.g., barcoded, labelled, indexed) suitable for DS. The cell index 16 provides a labelling tool that may identify or aid in identifying the cell from which the gDNA and/or the cDNA originated. In certain embodiments, cell indexed nucleic acid molecules can be extracted from the cells 12 and mixed together and sequenced (e.g., using NGS), such that data can be gathered regarding, for example, gDNA variants and RNA expression at the level of a single cell.

The term “binding” is used broadly throughout this disclosure to refer to any form of attaching or coupling two or more components, entities, or objects. For example, two or more components may be bound to each other via chemical bonds, covalent bonds, ionic bonds, hydrogen bonds, electrostatic forces. Van der Waals forces, Watson-Crick hybridization, etc.

The pooled cells 14 can be separated into different reaction vessels or containers and a first set of nucleic acid barcodes can be added to the plurality double-stranded nucleic acid molecules. As described herein, methods can include iterative rounds of pooling and splitting cells, wherein each splitting of the cells further comprises adding additional barcode sequences such that indexed nucleic acid molecules are likely bound to a unique cell index or label (e.g., combination of barcodes).

In embodiments, the methods of labeling nucleic acids in a plurality of cells may comprise fixing the plurality of cells prior to splitting the cells. For example, components of a cell may be fixed or cross-linked such that the components are immobilized or held in place. The plurality of cells may be fixed using formaldehyde in phosphate buffered saline (PBS). The plurality of cells may be fixed, for example, in about 4% formaldehyde in PBS. In various embodiments, the plurality of cells may be fixed using methanol (e.g., 100% methanol). In yet various other embodiments, the plurality of cells may be fixed using ethanol (e.g., about 70-100% ethanol). In still various other embodiments, the plurality of cells may be fixed using acetic acid. In various other embodiments, the plurality of cells may be fixed using acetone. Other suitable methods of fixing the plurality of cells are also within the scope of this disclosure.

In certain embodiments, the methods of labeling nucleic acids in a plurality of cells may comprise permeabilizing the plurality of cells prior to cell indexing. For example, holes or openings may be formed in outer membranes of the plurality of cells. TRITON™ X-100 may be added to the plurality of cells, followed by the addition of HCl to form the one or more holes. About 0.2% TRITON™ X-100 may be added to the plurality of cells, for example, followed by the addition of about 0.1 N HCl. In certain other embodiments, the plurality of cells may be permeabilized using ethanol (e.g., about 70% ethanol), methanol (e.g., about 100% methanol), Tween 20 (e.g., about 0.2% Tween 20), and/or NP-40 (e.g., about 0.1% NP-40). Other suitable methods of permeabilizing the plurality of cells are also within the scope of this disclosure. In various embodiments, the methods of labeling nucleic acids in cells may comprise fixing and permeabilizing the plurality of cells prior to cell indexing.

In embodiments, nuclei are removed from cells following fixation and prior to permeabilization. Nuclei are pooled and permeabilized as discussed above. In such embodiments, cell indexing steps (e.g., addition of barcodes) are performed within nuclei. Where examples are discussed herein with regard to cell indexing and labelling of cellular nucleic acid material within cells, one skilled in the art will recognize that such method steps can also take place in extracted/isolated nuclei.

In embodiments, the cells may be adherent cells (e.g., adherent mammalian cells). Fixing and/or permeabilizing may be conducted or performed on adherent cells (e.g., on cells that are adhered to a plate). For example, adherent cells may be fixed and/or permeabilized followed by trypsinization to detach the cells from a surface. Alternatively, the adherent cells may be detached prior to the separation and/or indexing steps. In some other embodiments, the adherent cells may be trypsinized prior to the fixing and/or permeabilizing steps.

In embodiments, unfragmented gDNA can be fragmented into smaller gDNA fragments that can be cell indexed and/or prepared for NGS sequencing. When in fixed cells, gDNA is associated with proteins (e.g., nucleosomes, etc.). In various embodiments, protein (e.g., nucleosomes) are depleted and gDNA can be fragmented. In various embodiments, methods can include use of reagents or tools for fragmenting double-stranded DNA, such as by enzymatic means (e.g., enzymes for random or semi-random genomic shearing and appropriate reaction buffers and reagents). Steps for enzymatically fragmenting double-stranded DNA can include providing one or more of enzymes for random or targeted digestion (e.g., restriction endonucleases. CRISPR/Cas endonuclease(s) and RNA guides, and/or other targeted endonucleases), double-stranded Fragmentase cocktails, single-stranded DNase enzymes (e.g., mung bean nuclease, Sl nuclease) for rendering fragments of DNA predominantly double-stranded and/or destroying single-stranded DNA, and appropriate buffers and solutions to facilitate such enzymatic reactions. In another embodiment, nucleosomes may not be depleted prior to transcriptome mediated adapter integration. As such, single cell DS can provide information related to chromatin accessibility, the concept of chromatin accessibility being as is known in the art (see, e.g., ATAC-seq methods, including for example Buenrostro et al., 2013. Transposition of native chromatin for fast and sensitive epigenomic profiling of open chromatin, DNA-binding proteins and nucleosome position. Nature Methods. 10 (12); 1213-8, which is incorporated by reference herein in its entirety for all purposes).

In embodiments. RNA molecules (e.g., RNA transcripts) can be reverse transcribed into cDNA following fragmentation of double-stranded gDNA. In embodiments, the reverse transcription primer may be configured to reverse transcribe all, or substantially all, RNA in a cell (e.g., a random hexamer with a 5′ overhang). In some other embodiments, the reverse transcription primer may be configured to reverse transcribe RNA having a poly (A) tail (e.g., a poly (dT) primer, such as a dT (15) primer, with a 5′ overhang). In yet some other embodiments, the reverse transcription primer may be configured to reverse transcribe predetermined RNAs (e.g., a transcript-specific primer). For example, the reverse transcription primer may be configured to barcode specific transcripts such that fewer transcripts may be profiled per cell, but such that each of the transcripts may be profiled over a greater number of cells.

In embodiments, and following cell preparation steps such as fixing, permeabilizing. DNA fragmenting, and/or reverse transcribing RNA molecules, steps for cell indexing can result in double-stranded DNA molecules labeled in a manner illustrated in FIG. 3B. Once split, the cells of each aliquot are provided a corresponding unique aliquot-specific barcode nucleic acid (e.g., aliquot-specific barcode 19). which binds (e.g., via ligation) to A-tailed genomic DNA (gDNA) of the cells of the aliquot. In further steps, cells are subsequently re-pooled (e.g., pooled cells 14), and then split again and the process repeated for ligating the subsequent corresponding aliquot-specific barcode (e.g., aliquot-specific barcode 20). This iterative process is repeated a sufficient number of times until each cell, or most cells, of the plurality of cells Il contains a unique (or almost unique) cell index 16 that includes a unique (or almost unique) combination of the corresponding aliquot-specific barcode sequences 19, 20, 21. Splinting oligonucleotides 22, are used to provide a substrate for annealing of the corresponding aliquot-specific barcodes. In the shown embodiment, a strand-defining element (SDE) is provided as a base pair mismatch 18, as further described herein. Accordingly, in various embodiments, methods include introducing a cell index 16 to nucleic acid molecules originating from the cells 11 according to a method of cell indexing, the method of cell indexing comprising; dividing an initial pool 14 of the cells into a plurality of aliquots 13, wherein cell membranes of the cells retain cellular nucleic acid molecules therein (or in another embodiment, nuclear membranes of the nucleus of the cell retains nuclear nucleic acid molecules therein); contacting an aliquot of the plurality of aliquots 13 with a corresponding aliquot-specific barcode (e.g., 19, 20, 21) such that the cellular nucleic acid molecules within cells split into any particular aliquot are ligated to the corresponding aliquot-specific barcode to form aliquot-barcoded cellular nucleic acid molecules within the cells; re-pooling the plurality of aliquots of cells to form a subsequent pool 14, wherein the subsequent pool serves as the initial pool of the cells for a subsequent repetition of these steps; and repeating these steps until most cells or every cell of the biological sample contains cellular nucleic acid molecules therein that are uniquely labeled according to the cell index 16. It is understood that the barcodes and other sequences depicted in FIG. 3B and elsewhere are illustrated with an arbitrary number of bases (represented by “Ns” or another variable). As the number of “Ns” or other variable depicted are only exemplary, it is recognized that the number of bases of an oligo, a barcode, a sequence region, an adapter, etc., may have fewer or more than the number of bases shown.

FIGS. 3C-3D are conceptual illustrations of various single-cell DS method steps using the combinatorial cell indexing scheme of FIGS. 3A-3B, and in accordance with an embodiment of the present technology. For example, FIGS. 3C and 3D illustrate a method (e.g., Steps 1-7) for preparing a sequencing library suitable for single cell DS. In embodiments, such a sequencing library is suitable for detection of low-frequency variants in cells. In certain embodiments, cells are fixed and permeabilized such that the cell membranes of the cells retain cellular nucleic acid molecules therein, and cellular gDNA are subjected to depletion of nucleosomes 24 (FIG. 3C, Step 1) prior to fragmentation of the genome to produce genome fragments 17 (FIG. 3C, Step 2), e.g., cellular nucleic acid molecules and/or polynucleotide sequences (e.g., gDNA) 25 comprised of a top strand 25a and a bottom strand 25b. In embodiments, following gDNA fragmentation, the method can include reverse transcribing RNA to generate double-stranded cDNA molecules. The fragmented gDNA and generated cDNA molecules can be subject to the combinatorial indexing steps described below.

While it is understood that a plurality of gDNA fragments and/or cDNAs will be labelled as part of the combinatorial cell indexing method, Steps 3-7 illustrate the steps showing the process for one gDNA molecule. As part of combinatorial indexing (cell indexing), cellular nucleic acid molecules 25 can be ligated to a cell indexing adapter 27 with a corresponding aliquot-specific barcode that comprises a base pair mismatch for the SDE 18 (e.g., “Wobble base”, shown as GG< >TT for exemplary purposes) and an index (e.g., labeled as “cellular index”) as an aliquot-specific barcode 19. Between Step 3 and Step 4, additional rounds of iterative cell indexing can occur. For example, cells provided with aliquot-specific barcode 19 can be re-pooled with other split cells and a first splinting oligonucleotide 22 can be provided to the re-pooled cells where splinting oligo 22 will hybridize to a single-stranded overhang of cell indexing adapter 27. Next, cells can be split again into a plurality of aliquots wherein an additional aliquot specific barcode 20 molecule hybridizes with a single-stranded overhang of the splinting oligo 22 (see Step 4). Re-pooling of the split cells can occur (not shown) where splinting oligo 23 can be provided for hybridization to a single-stranded overhang of the aliquot specific barcode 20 molecule. Further splitting of cells into yet another plurality of aliquots allows for introduction of aliquot-specific barcode 21 molecule which hybridizes with a single-stranded overhang of splinting oligo 23 (see, e.g., Step 4). In embodiments, a ligase enzyme is used to covalently link the cell indexing adapter 27, the aliquot-specific barcode 20 molecule, and the aliquot-specific barcode 21 molecule. Accordingly, with use of splinting oligonucleotides (e.g., splinting oligonucleotide 22. 23), and after two additional rounds of combinatorial indexing, the cellular nucleic acid molecules 25 are labeled with aliquot-specific barcodes 19. 20, and 21, resulting in a unique (or almost unique) label or tag (“cell index”) for nucleic acid molecules from each or most cells of the plurality of cells, as shown at FIG. 3C. Step 4. In the illustrated embodiment, aliquot-specific barcode 21 comprises a unique molecular identifier (UMI, 21a) as a sequence element of the barcode, however, the UMI can be introduced at any step of the process without departing from the scope of the disclosure. While three rounds of splitting and barcoding are illustrated in FIG. 3C, one skilled in the art will recognize that fewer rounds or more rounds of combinatorial cell indexing may be used. For example, the number of rounds of combinatorial cell indexing may depend on the total number of pooled cells; the larger number of pooled cells, the greater the need for additional rounds of cell indexing to provide a substantially unique cell identifier/index.

In embodiments, a cell indexing adapter 27 is introduced to the cellular nucleic acid molecules 25 by contacting the biological sample comprising the cellular nucleic acid molecules with the cell indexing adapter 27 (FIG. 3C, Step 3). In embodiments, the cell indexing adapter may include a sample index sequence (not shown), such that the cellular nucleic acid molecules within the plurality of cells belonging to a same biological sample are ligated to the cell indexing adapter 27 having a sample-specific barcode to form sample-barcoded cellular nucleic acid molecules. In embodiments, the sample-specific barcode can be added to nucleic acid molecules prior to splitting the cells. In other embodiments, the first aliquot specific barcode 19 can be the sample index. As shown, cell index adapter 27 can provide the SDE 18 and aliquot-specific barcode 19 as discussed. However, the SDE can be introduced to the cellular nucleic acid molecules by other methods without departing from the scope of the disclosure. In embodiments. and as shown at FIG. 3C, the UMI 21a can be introduced to the cellular nucleic acid molecules by way of a cell indexing round (e.g., shown as a third round) in conjunction with the aliquot-specific barcode 21.

With reference to FIGS. 3C and 3D (following Step 4), and in conjunction with an embodiment, the cells can be lysed and the preliminary nucleic acid sequencing library comprising a plurality of cell indexed gDNA and cDNA molecules can be extracted, purified (e.g., via streptavidin beads or other DNA purification method) and pooled for further processing. For example, FIG. 3D, Step 5 illustrates a cell indexed gDNA molecule following polymerase extension or gap fill-in (e.g., with BST or other polymerase) to form a double-stranded complex (cell indexed cDNA molecules, not shown, can also be subject to Step 5). As shown, polymerase extension converts the UMI 21a to a double-stranded form of the UMI (e.g., FIG. 3D, Step 5). In other embodiments, a UMI can be introduced to the cellular nucleic acid molecules by other methods (e.g., primer-based UMI added during primer extension) without departing from the scope of the disclosure. Following Step 5, cell indexed cDNA molecules can be separated from cell indexed gDNA molecules as is known in the art and described further herein. As such, cell indexed cDNA molecules can be amplified and bulk sequenced separately.

Following primer extension and/or gap fill-in (FIG. 3D, Step 5), the double-stranded indexed gDNA molecule comprises the gDNA sequence 25, base pair mismatch 18, and barcodes 19, 20, and 21 including the UMI 21a. Although only depicted on one end of the gDNA fragment, a combinatorial cell index can be provided at both ends of any gDNA fragment.

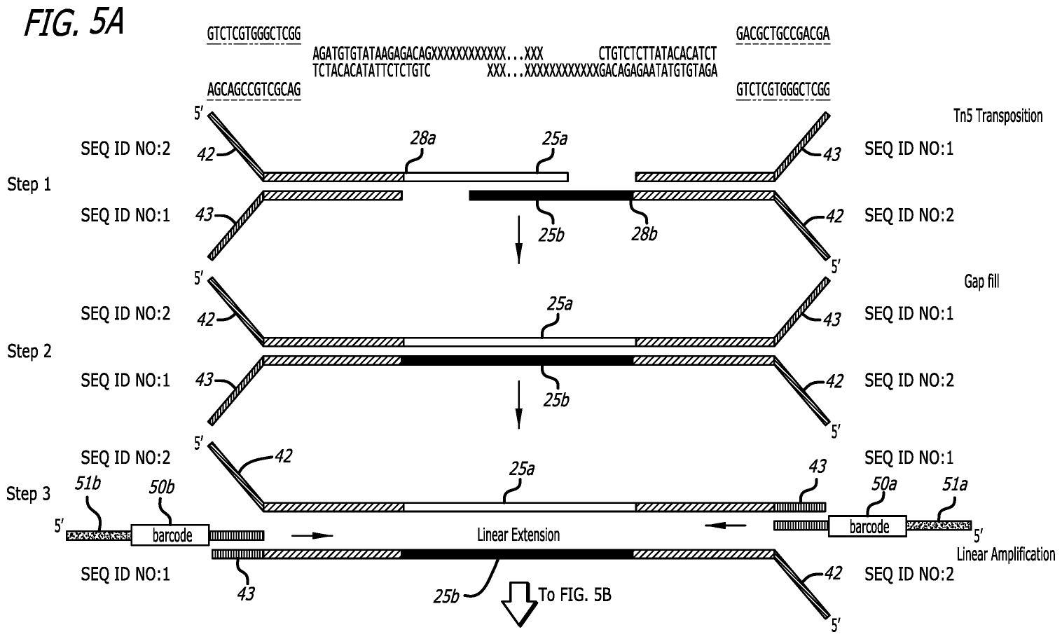

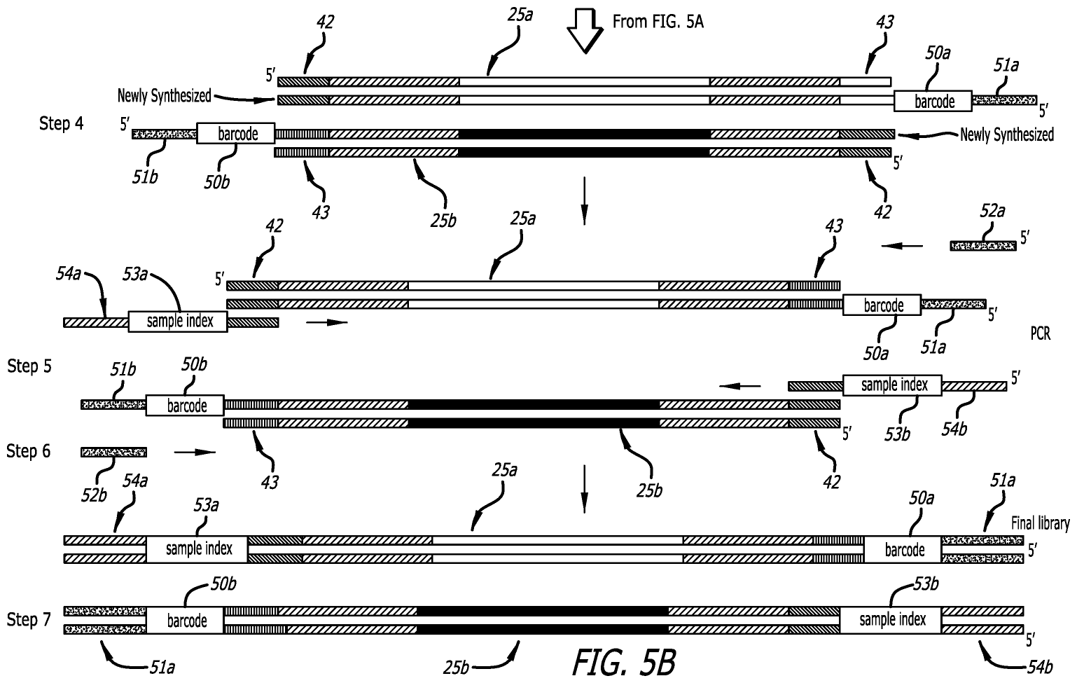

At FIG. 3D. Step 6, a second fragmentation step is performed to further fragment the gDNA sequence 25 (e.g., to generate suitable fragment sizes for NGS sequencing). In embodiments, double-stranded cell-indexed gDNA molecules can be enzymatically fragmented as discussed above (e.g., with enzymes for targeted digestion (e.g., restriction endonucleases, CRISPR/Cas endonuclease(s) and RNA guides, and/or other endonucleases), double-stranded Fragmentase cocktails, single-stranded DNase enzymes (e.g., mung bean nuclease, SI nuclease) for rendering fragments of DNA predominantly double-stranded and/or destroying single-stranded DNA, and appropriate buffers and solutions to facilitate such enzymatic reactions). In embodiments, the method further comprises ligating sequencing adaptors 26 to the cellular nucleic acid molecules for DS thereby generating double-stranded cell-indexed gDNA complexes. For example, following the second fragmentation step, the method can include ligation of the cell indexed gDNA molecules to platform-specific adapters, for example, Illumina® adaptor (Index 2 or Index I adaptor; i7 or i5 adaptor) 26, on each end of the double-stranded molecule (as shown at FIG. 5D. Step 6). In embodiments, platform specific adapters having amplification primer binding sites may be ligated such that additional platform specific sequences (e.g., P5/P7 as shown at FIG. 3D, Step 7) may be added through an amplification step (e.g., through PCR amplification using primers that comprise adapter sequences, thereby producing the adapter sequences of 26).

As shown at FIG. 3D, Step 7, the method includes PCR amplification of the double-stranded cell-indexed gDNA complex to generate a first strand amplicon family and a distinct yet related second strand amplicon family. For example, each family of the pair of molecular families results from amplification of one of the two strands of the original cellular nucleic acid molecule 25. In embodiments, the probability of a technical variant or sequencing error occurring during NGS workflows at the same base position in each family of the two families is very low, and therefore sequence variants that are detected in only one of the two families can be identified as technical variants or sequencing errors during data analysis. Sequence variants that occur at the same base position in each family of the two families may be identified as biological variants during data analysis.

As discussed above, single-cell DS may include additional steps to provide cell-specific or cell-identifiable information. For example, after gDNA fragmentation at FIG. 3C, Step 2, the method may also include reverse transcribing RNA to generate cDNA molecules, cDNA molecules may then be processed as discussed above in the same process steps as gDNA 25. For example, both cDNA and gDNA molecules can be subject to sequencing library preparation. In embodiments, and after cell lysis (e.g., following FIG. 3D, Step 4), cDNA and gDNA molecules are extracted and prepared for sequencing. In other embodiments, cDNA molecules can be pulled out via biotinylated reverse transcription (RT) primer and streptavidin beads can be used separate cell-indexed cDNA molecules from cell-indexed gDNA molecules such that cDNA molecules may be sequenced in a different manner (e.g., NGS sequencing without DS). In additional embodiments, RT primers can have a specific “RT tag” (not shown) of defined/known sequence that is incorporated during reverse transcription that indicates a particular sequence read is derived from cDNA (e.g., rather than gDNA). Sequencing results from cDNA molecules can provide transcript or transcriptome information that identifies cell specific information, including but not limited to cell type, cell state, cell origin, etc. Because both gDNA duplex families (e.g., derived from a first/top stand and from a second/bottom strand) contain the same cell index (e.g., 19, 20, 21), the biological variants are traceable to individual cells and cell type may confirmed via cDNA analysis.

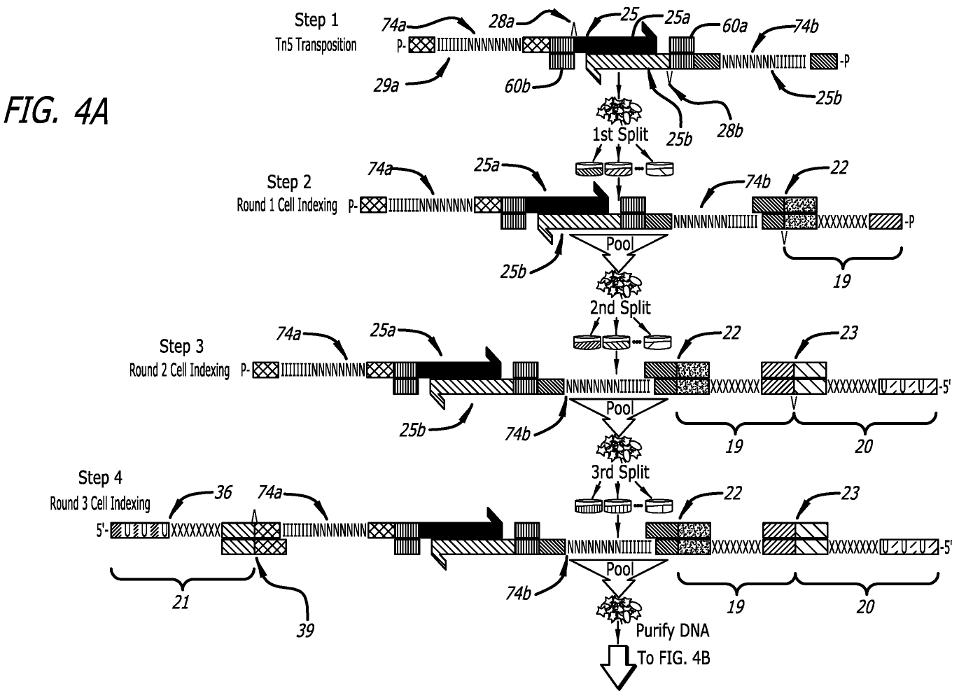

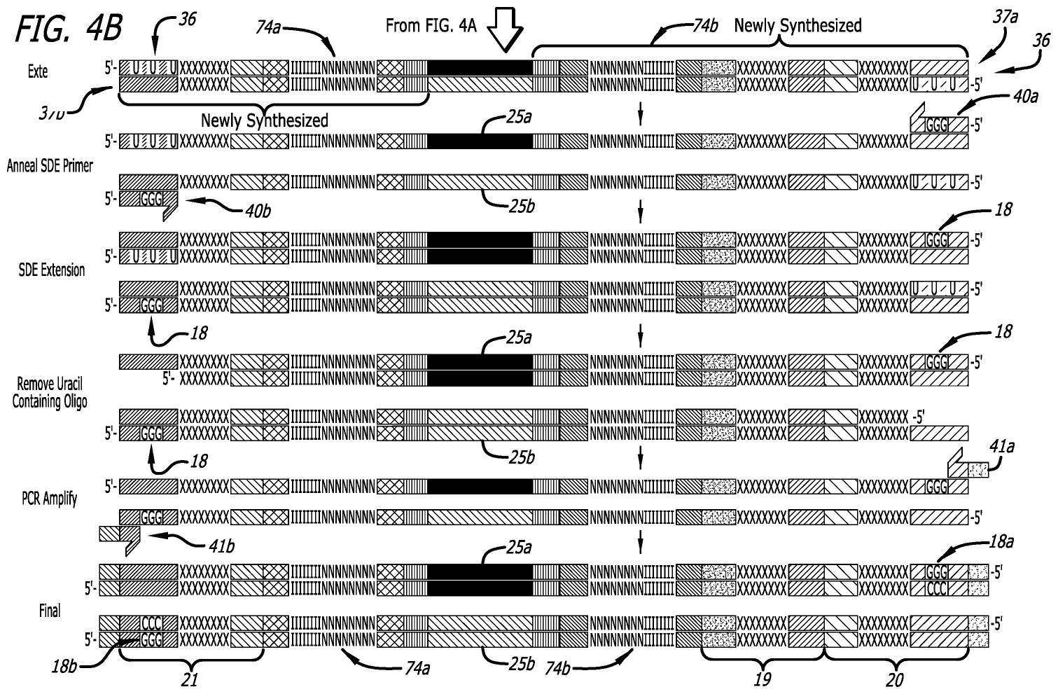

FIGS. 4A-4B are conceptual illustrations of various single-cell DS method steps using a combinatorial cell indexing scheme as discussed above and utilizing transposon-mediated adapter integration, in accordance with another embodiment of the present technology. In reference to the FIGS. 4A-4B the “IIIIIIII” refers to “Sample index”, “NNNNNNNN” refers to “UMI”, and “XXXXXXXX” refers to “Aliquot-specific barcode”. Newly synthesized strand elements are denoted in brackets.

In embodiments, attachment of the adapter is achieved by or in conjunction with at least one of a transposase enzyme belonging to the DDE Transposase family (e.g., this family includes, but is not limited to, the maize Ac transposon, Drosophila P element, bacteriophage Mu, Tn3. Tn21, Tn1721, Tn2501, Tn3926, Tn5, Tn10, Mariner, IS10, IS50, etc.) Listings of DDE Transposase family enzymes are available in the publicly available literature in both printed and computer readable forms. It will be appreciated by one of ordinary skill in the art that any DDE transposase family enzyme may be used in accordance with various embodiments of the present technology. In other embodiments, a targeted transposase is or comprises at least one of a ribonucleoprotein complex, such as, for example, a CRISPR-associated (Cas) enzyme/guide RNA complex (e.g. Cas12k) or Cas12k-like enzyme. In other embodiments, this targeted transposase and ribonucleoprotein complex belongs to the IS200/605 transposon family. A listing of targeted transposons can be obtained in Altae-Tran H. Kannan S, et al. Science 374 (6563), 57-65;2021 (incorporated herein by reference). It will be appreciated by one of ordinary skill in the art that any member of the IS200/605 transposon family could be used in accordance with various embodiments of the present technology. In embodiments, a targeted transposase or a mixture of targeted transposases may be used to attach an adapter at more than one potential target region of a nucleic acid material (e.g., 2, 3, 4, 5, 6, 7, 8, 9, 10 or more). In embodiments, where there is more than one target region of a nucleic acid material, each target region may be of the same (or substantially the same) length. In embodiments, where there is more than one target region or a nucleic acid material, at least two of the target regions of known length differ in length (e.g., a first target region with a length of 100 bp and a second target region with a length of 1,000 bp). Certain embodiments presented herein describe use of a Tn5 transposase, however one of ordinary skill in the art will recognize that this is for illustrative purposes and that additional transposase enzymes, such as those described here, may also be suitable for use in the described methods.

Referring to FIGS. 4A and 4B together, and following steps described above with respect to fixing and permeabilizing cells and/or organelles thereof such that cell membranes (or nuclear membranes) of the cells retain cellular nucleic acid molecules therein, a barcode nucleic acid molecule 74 is covalently attached via transposase activity, to cellular nucleic acid molecules 25 within cells of a biological sample to form barcoded cellular nucleic acid molecules (FIG. 4A, Step 1). One of ordinary skill in the art will appreciate that any suitable transpose may be used. For purposes of illustration only, the examples described herein refer to a Tn5 transposase and suitable system for using a Tn5 transposase. For example, transposition of adapter molecules 29a, 29b to double-stranded nucleic acid molecules 25 can occur at Tn5 transposition sites 28a and 28b.

In the embodiment illustrated in FIG. 4A, a combinatorial cell indexing method is performed in Steps 2-4, to form cell indices, followed by DNA extraction and purification from the cells for further sequencing library preparation steps (FIG. 4B). The cell index enables cellular nucleic acid molecules derived from a particular cell to be identified following sequencing and data analysis. While any method of cell indexing can be used following adapter attachment via transposase, in certain embodiments and as shown in FIGS. 4A and 4B, the method of cell indexing comprises dividing an initial pool of the cells into a plurality of aliquots (e.g., 1st split of FIG. 3A. Step 2). Addition of splinting oligonucleotide 22 allows for hybridization with the single-stranded overhang of adapter molecule 29b. Splinting oligonucleotide 22 allows the hybridization of aliquot-specific barcode molecule 19 to each molecule within the specific aliquot. In Step 3, cells are re-pooled from the plurality of aliquots of cells to form a subsequent pool, wherein the subsequent pool serves as the initial pool of the cells for a subsequent repetition of these steps (e.g., 2^{nd} split, 3^{rd} split, and corresponding pooling steps of FIG. 4A, Steps 3 and 4). For example, in the 2^{nd} split (Step 3), splinting oligonucleotide 23 provides the hybridization bridge for aliquot-specific barcode molecule 20 comprising a uracil-containing element a 5′ end (and which is associated with the bottom strand 25b of the cellular nucleic acid molecule 25). In the illustrated 3^{rd} split (Step 4), splinting oligonucleotide 39 provides the hybridization bridge for aliquot-specific barcode molecule 21 comprising a uracil-containing element a 5′ end (and which is associated with the top strand 25a of the cellular nucleic acid molecule 25). These steps are shown in the example as being performed three times, however, a skilled artisan will appreciate that these steps can be repeated fewer times or more times until most cells or every cell of the biological sample contains cellular nucleic acid molecules therein that are uniquely (or nearly uniquely, e.g., about 100%, about 99%, about 98%, about 97%, about 96%, about 95%, about 90%, about 85%, about 80%, about 75%, about 70%, about 65%, about 60%, about 55%, or about 50% uniquely) labeled according to the cell index.

Following Step 4, cells may be re-pooled, lysed and the cell indexed nucleic acid molecules (e.g., indexed gDNA or cDNA molecules) may be purified as is known in the art and described herein. Following target nucleic acid extraction, and as shown at FIG. 4B, Step 5, polymerase extension with a uracil tolerant polymerase can be performed to generate double-stranded cell-indexed DNA molecules. In embodiments, removal of splinting oligos can be achieved through use of a strand displacing polymerase or, in another embodiment, through use of a polymerase with 5′-3′ exonuclease activity. Alternatively, the structures can be made double-stranded through use of polymerase gap fill and ligation with the splinting oligos.

At Steps 6 and 7, cell-indexed DNA molecules can be melted and primer annealing and polymerase extension with an SDE primer 40a (e.g., making a copy of the top strand 25a) and SDE primer 40b (e.g., making a copy of the bottom strand25b) is performed to provide an SDE for each individual strand 25a, 25b. As illustrated, SDE primers 40a and 40b anneal to the non-uracil containing elements 37a and 37b, respectfully (Step 6). As shown, SDE primers 40a and 40b comprise base mismatches (e.g., “GGG”) relative to the non-uracil containing elements 37a, 37b of the cell indexed nucleic acid molecules 25 and which will used as the SDE 18 during data analysis.

At Step 7, the method includes extending. with a DNA polymerase, from the SDE primer 40a or 40b to form a copy strand complexed with the original template strand, such that the copy strand comprises the base pair mismatch (FIG. 4B. Step 7). While the SDE is shown as being introduced with use of the SDE primers 40a and 40b, the SDE can be introduced with other methods without departing from the scope of the disclosure.

In Step 8, the resulting double-stranded cell indexed acid molecules are enzymatically treated to remove the uracil-containing elements 36 from the original cell-indexed strands. As such, the uracil-containing element 36 is removed and/or destroyed (e.g., made inoperable for primer binding or future copying) from both the top and bottom strand molecules. In certain embodiments, the uracil-containing element 36 can be destroyed using a uracil DNA glycosylase (UDG) or a mixture of uracil DNA glycosylase and single-strand endonuclease, such as endonuclease III or endonuclease VIII or endonuclease IV (e.g., USERR Il Enzyme (New England Biolabs. M5508S or M5508L)). In other embodiments, not shown, a different element that could be made to be inoperable could be used. For example, an element incorporating inosine instead of uracil could be used and the element could be removed or destroyed through use of an enzyme from the DNA-deoxyinosine glycosylase family of enzymes. In embodiments, use of and subsequent removal of the uracil-containing elements allows for filtering or removing from processing of the original nucleic acid strands that do not yet have an SDE. Alternatively, sequence reads not having an SDE can be removed during data analysis.

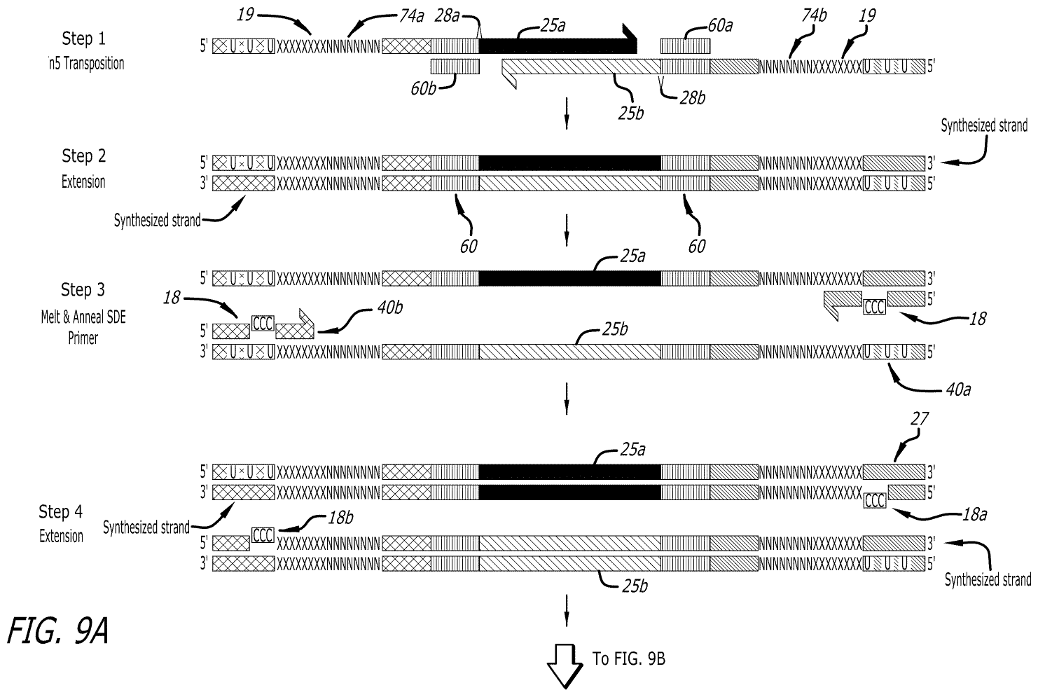

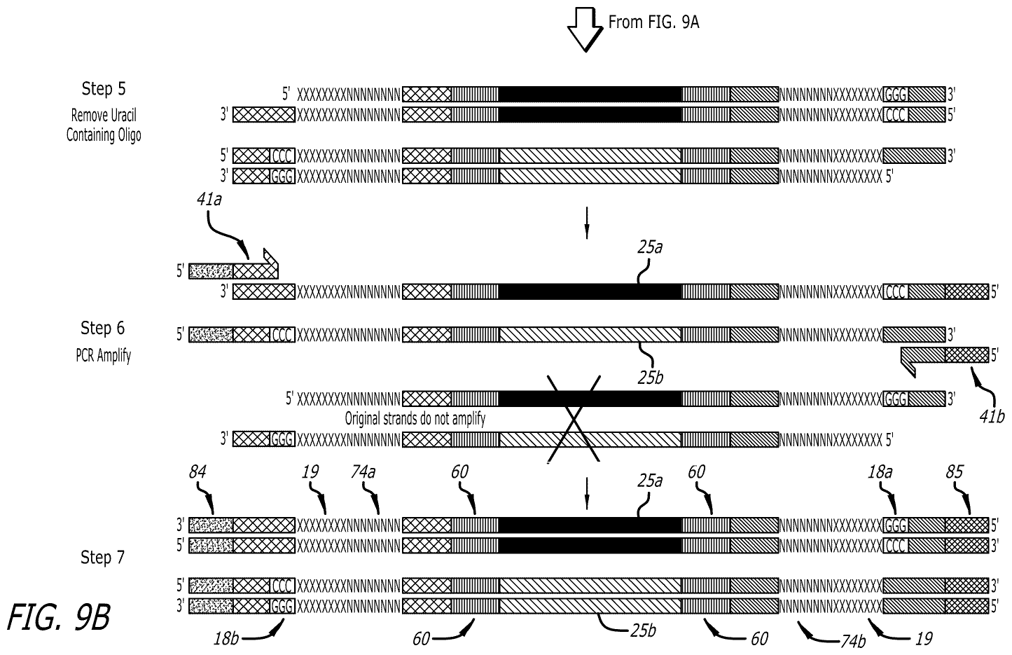

With reference to FIG. 4B, Step 9, the method includes providing primers 41a and 41b which anneal to the 3′ ends (e.g., non-uracil containing elements 37a, 37b) of the “copy” strands. As shown, primers 41a and 41b include primer tails providing platform-specific adapter sequences (e.g., . Illumina® adaptor (Index 2 or Index 1 adaptor; i7 or i5 adaptor sequences). With reference to Steps 9) and 10, polymerase extension from annealed primers 41a and 41b provide double stranded adapter-nucleic acid molecules wherein each nucleic acid fragment 25a, 25b has flanking adapter regions together comprising UMI sequences 74a. 74b, sample index barcodes 19, strand index as SDE 18a. 18b, and primer binding sites (for further exponential amplification) and/or flow cell binding sites (FIG. 9B, Steps 9 and 10). In other embodiments, the method may include ligating sequencing adaptors (e.g., comprising platform-specific adapter sequences) to the cell indexed nucleic acid molecules to configure the cell-indexed cellular nucleic acid molecules for NGS sequencing.

The resultant sequencing library as prepared through transposon-mediated fragmentation and adapter integration followed by combinatorial cell indexing provides a sample index (for differentiating nucleic acids within multiplexed samples), cell indexing (for differentiating cellular nucleic acids derived from individual cells from other cells in a population), molecular indexes (for differentiating molecules from other molecules in a cell and/or sample), and a strand index for differentiating sequencing information derived from each of the two strands to facilitate sequence comparison from the two strands of an original molecule for duplex sequence error correction. In embodiments not shown, the above transposon-mediated tagging scheme can be used without the use of an additional UMI sequence (e.g., 74a. 74b). In such embodiments, information related to fragment ends/transposition sites, mosaic elements (ME) or a combination thereof can be used as a molecular index.

As shown at FIG. 4B, Step 10, a prepared sequencing library contains distinct yet related molecular families derived from each of the original first strand (e.g., top strand, Watson strand) and original second strand (e.g., bottom strand, Crick strand). As such, each family of the pair of molecular families results from amplification of one of the two strands of the original cellular nucleic acid molecule 25. The probability of a technical variant or sequencing error occurring during NGS workflows at the same base position in each family of the two families is very low, and therefore sequence variants that are detected in only one of the two families may be identified as technical variants or sequencing errors during data analysis. In other embodiments. differences in sequence between the two strands may be a result of an in vivo condition where cellular DNA repair processes have not yet cured particular mismatches. However, sequence variants that occur at the same base position in each family of the two families may be identified as true biological variants during data analysis. Because both families contain the same cell index (e.g., 19, 20, 21), the biological variants are traceable to individual cells as discussed above.

Accordingly, following sequencing and data analysis of the sequencing library prepared according to the steps of FIGS. 4A and 4B, nucleic acid molecules of the sequencing library comprise a sample index (e.g., IIIIIIII), a cell index (e.g., combination of 19, 20, 21), a molecular index (e.g., NNNNNNNN), and a strand index (e.g., SDE 18). In various embodiments, the sample index is for identification of a particular biological sample among biological samples, the cell index is for identification of a particular cell among cells, the molecular index is for identification of a particular cellular nucleic acid molecule among cellular nucleic acid molecules, and the strand index is for identification of a particular strand of the particular cellular nucleic acid molecule.

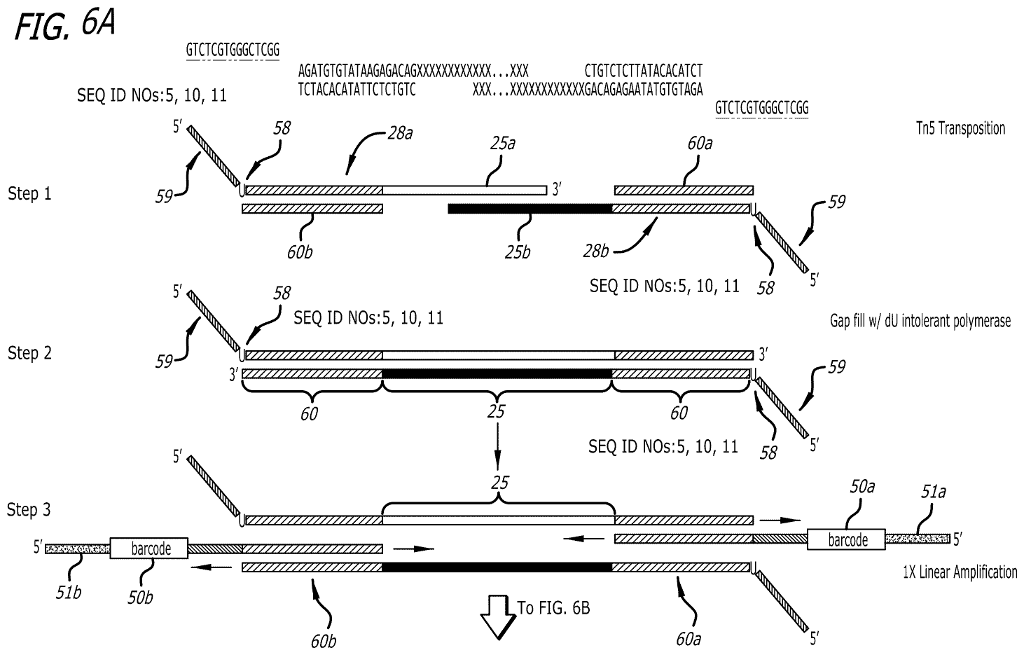

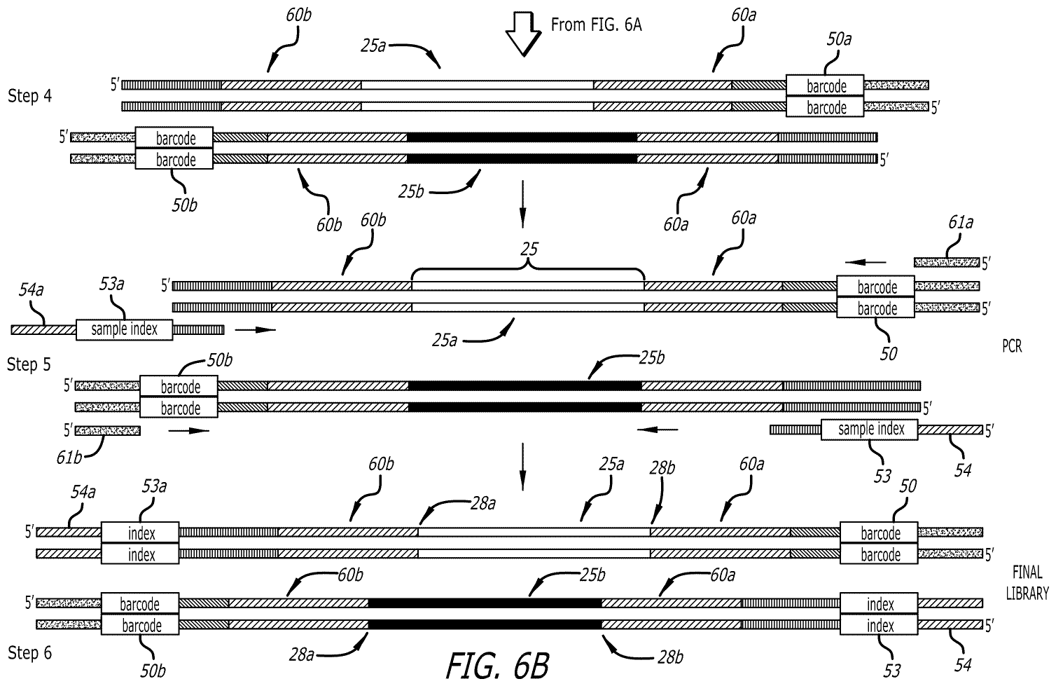

FIGS. 5A and 5B and FIGS. 6A and 6B are conceptual illustrations of various single-cell DS method steps utilizing a transposase for adaptor integration and in accordance with yet another embodiment of the present technology. In the drawings, “XXX. . . . XXX” refers to genomic DNA (gDNA). The embodiments described in FIGS. 5A-6B are suitable for and make use of systems of single-cell interrogation utilizing microfluidic droplets (e.g., monodisperse emulsion microdroplets) containing primers with droplet-specific barcodes. In such embodiments, the droplet-specific (e.g., cell-specific) barcode is an oligonucleotide that can be affixed to a bead or a solid surface and provided into the emulsion microfluidic droplet. The barcode sequence may contain a plurality of bases and should contain enough bases to provide a sufficiently unique cell index for the number of cells being interrogated. In embodiments, the droplet-specific barcode at least 6 nucleotides, at least 8 nucleotides, at least 10 nucleotides, at least 12 nucleotides, at least nucleotides, or at least 16 nucleotides. Such systems are described in Zheng, et al., (Massively parallel digital transcriptional profiling of single cells. Nat Commun. 2017;8;14049. doi; 10.1038/ncomms14049), which is incorporated herein by reference in its entirety. As used herein for RNA as well as gDNA fragments, a UMI can be “assigned” the genomic coordinates at which two consecutive Tn5 transposition events occur resulting in a DNA fragment in a particular partitioned cell or nuclei. In these embodiments, the coordinates at which Read 1 and Read 2 start at for a given pair of genomic coordinates and at which two consecutive Tn5 insertions gave rise to fragment of DNA can be used an SDE. Said a different way, the orientation of the cell index barcode and the genomic transposition site (e.g., mapping coordinates with respect to a reference sequence) is different between the top strand and the bottom strand and becomes possible to differentiate the strands. For example, one strand would have (sequencing) Read I coordinates <Read 2 coordinates, whereas the other strand would have Read 2 coordinates <Read 1 coordinates in a given cell. As it is unlikely that any two transposition events would occur at the same genomic position/coordinates in both chromosomes of a diploid genome within the same cell, this information becomes a unique label for distinguishing molecules from each other and from differentiating strands of any particular molecule.

Referring now to FIGS. 5A and 5B together, method steps are illustrated for preparing a sequencing library (e.g., for single cell DS), utilizing schemes for adaptor integration and attachment via a transposase and utilizing orientation of barcode(s) for the strand-defining element (SDE). With reference to FIG. 5A, Step 1, and within cells or nuclei partitioned in microfluidic droplets as described above. Tn5 transposition can be used to bind and/or integrate sequencing adaptors 42 and 43 to cellular nucleic acid molecules 25a and 25b at Tn5 transposition sites 28a and 28b. This step functions to fragment double-stranded gDNA. In Step 2, gap filling can produce two complementary strands.

In the illustrated embodiment, a first nucleic acid molecule with barcode 50a, 50b having a 5′ single-strand overhang 51a or 51b is annealed to a 3′ end of the first sequencing adaptor 43 (FIG. 5A, Step 3) and linear extension via polymerase generates a nucleic acid complex comprising a cell index (FIG. 5B, Step 4). As such, DNA polymerase extension from a 3′ end of the first barcode nucleic acid molecule (50a/50b and 51a/51b), produces complementary or “copy” strands with 5′ overhangs 51a and 51b. The barcode may be any length suitable to provide diversity of barcodes to be able to identify nucleic acid molecules as derived from a particular cell. In embodiments, the barcode is between about 10-30 bases long, between about 10-50 bases long, about 45 bases, about 16 bases, etc.

With reference to FIG. 5B, Step 5 illustrates melting of the original adapter-strand and the cell-indexed adapter strand followed by annealing of oligonucleotides comprising a sample index sequence 53a, 53b. The sample index oligos 53 with 5′ single-strand overhang 54a and 54b, respectively, can be annealed to a 3′ end of second sequencing adaptor 42 (FIG. 5B. Step 5). As illustrated in Step 6, extension with PCR using DNA polymerase and added primers 52a and 52b, and from a 3′ end of the second barcode 53a. 53b, can generate a final cellular nucleic acid sequencing library. In the final library, the SDE is determined according to an orientation of the sequencing adaptors (51a/51b, 50a/50b, 53a/53b, 54a/54b) of the adaptor-labeled cellular nucleic acid molecules with respect to a 5′ end or a 3′ end of the adaptor-labeled cellular nucleic acid molecules. In various embodiments, a polynucleotide sequence of the first barcode nucleic acid molecule 50a/50b is different from a polynucleotide sequence of the sample index 53a/53b.

In various embodiments, the sample index 53 is for identification of a particular biological sample among biological samples, the cell index (e.g., barcode 50) is for identification of a particular cell among cells, the molecular index (e.g., transposition sites 28a and 28b) is for identification of a particular cellular nucleic acid molecule among cellular nucleic acid molecules, and the strand index (barcode orientation differences between top and bottom strands) is for identification of a particular strand of the particular cellular nucleic acid molecule. Because both strands and derivatives thereof contain the same cell index (e.g., 50), the biological variants are traceable to individual cells as discussed above. For example, following transposase-mediated adapter integration within gDNA, steps for reverse transcription of RNA to cDNA can occur within the cells, cDNA can be separated from the gDNA fragments prior to sequencing via biotin/strepavidin pulldown or other technique known for separation of cDNA for transcriptome analysis. As cDNA are labelled with the same cell index/barcode 50, transcript information can be related to gDNA sequence information and variants detected by DS can be attributed to a particular cell or cell type.

Referring now to FIGS. 6A and 6B together, method steps are illustrated for preparing a sequencing library (e.g., for single cell DS), utilizing schemes for adaptor integration and covalent attachment with a transposase and utilizing orientation of barcode(s) for the strand-defining element (SDE) in accordance with another embodiment. As discussed above, cells or nuclei are separated from each other into microfluidic droplets (e.g., monodisperse emulsion microdroplets) for single cell labeling steps. With reference to FIG. 6A, Step 1, a first sequencing adaptor 59 is integrated (e.g., attached via transposition) to cellular gDNA molecules (25, 25a, 25b) within cells of a biological sample to form adaptor-labeled and fragmented gDNA molecules. As shown, the first sequencing adaptor 59 is attached to a single strand of a mosaic end (ME) element (e.g., 60. 60a, 60b) by a deoxyuracil (dU) residue.

As shown in Step 2, a partially double-stranded/partially single-stranded adapter-labeled gDNA molecule is generated following gap-filling with a dU-intolerant polymerase (Step 2). As shown, the dU-intolerant polymerase does not copy the first sequencing adapter 59 segments leaving these segments single-stranded. A skilled artisan would understand that a dU-intolerant polymerase is a polymerase that is unable to copy a uracil and is prevented from copying a template strand beyond an incorporated uracil. In embodiments, a dU-intolerant polymerase may include, but is not limited to, use of Q5R High-Fidelity DNA Polymerase, PhusionR High-Fidelity DNA Polymerase *. VentR DNA Polymerase, Vent® (exo-) DNA Polymerase, Deep VentR DNA Polymerase, and Deep VentR (exo-) DNA Polymerase (available at New England Biolabs. Ipswich, MA).

In Step 3, oligonucleotides comprising a first barcode nucleic acid molecule 50a, 50b and a second adaptor sequence (51a, 51b) that anneals to the ME (60, 60a. 60b) is provided and polymerase extension with a dU-tolerant polymerase is used to extend from a 3′ end of the first barcode nucleic acid molecule 50a, 50b and from a 3′ end of the ME (60a, 60b) to form a complementary first-barcoded nucleic acid molecules (FIG. 6B, Step 4). In embodiments a dU-tolerant polymerase may include, but is not limited to, use of Taq polymerase (and mixtures and/or modified versions, thereof), Bst DNA polymerase (and mixtures and/or modified versions), phi-29, E, coli DNA pol I (and it's derivatives), and Pfu DNA polymerase with mutation that removes dU intolerance (available as Thermo Scientific Phusion U DNA polymerase).

As illustrated, the method further comprises providing and annealing a nucleic acid oligonucleotide with sample index 53a, 53b to a 3′ end of the first-barcoded nucleic acid molecules (FIG. 6B, Step 5) and extending. with a DNA polymerase and primers 61a and 61b, from a 3′ end of the sample index molecule 53 to generate cell-and sample-barcoded nucleic acid molecules (FIG. 6B, Steps 5 and 6) to form an NGS sequencing library. In various embodiments, a polynucleotide sequence of the first barcode nucleic acid molecule 50a, 50b (e.g., cell index) is different from a polynucleotide sequence of the sample index 53a 53b.

In embodiments, the method of FIGS. 6A and 6B comprises introducing a strand index as a strand-defining element (SDE) to the cellular nucleic acid molecules 25a, 25b, such that the SDE is determined according to an orientation of the barcode (e.g., 50a/50b, 53a/b of FIG. 6B) of the adaptor-labeled cellular gDNA molecules with respect to a sequence (e.g., 25a. 25b) of the cellular gDNA molecules.

In various embodiments, the sample index 53 is for identification of a particular biological sample among biological samples, the cell index (e.g., barcode 50) is for identification of a particular cell among cells, the molecular index (e.g., transposition sites 28a and 28b) is for identification of a particular cellular nucleic acid molecule among cellular nucleic acid molecules, and the strand index (barcode orientation differences between top and bottom strands) is for identification of a particular strand of the particular cellular nucleic acid molecule. Because both strands and derivatives thereof contain the same cell index (e.g., 50), the biological variants are traceable to individual cells as discussed above. For example, following transposase-mediated adapter integration within gDNA, steps for reverse transcription of RNA to cDNA can occur within the cells, cDNA can be separated from the gDNA fragments prior to sequencing via biotin/streptavidin pulldown or other technique known for separation of cDNA for transcriptome analysis. As cDNA are labelled with the same cell index/barcode 50, transcript information can be related to gDNA sequence information and variants detected by DS can be attributed to a particular cell or cell type.

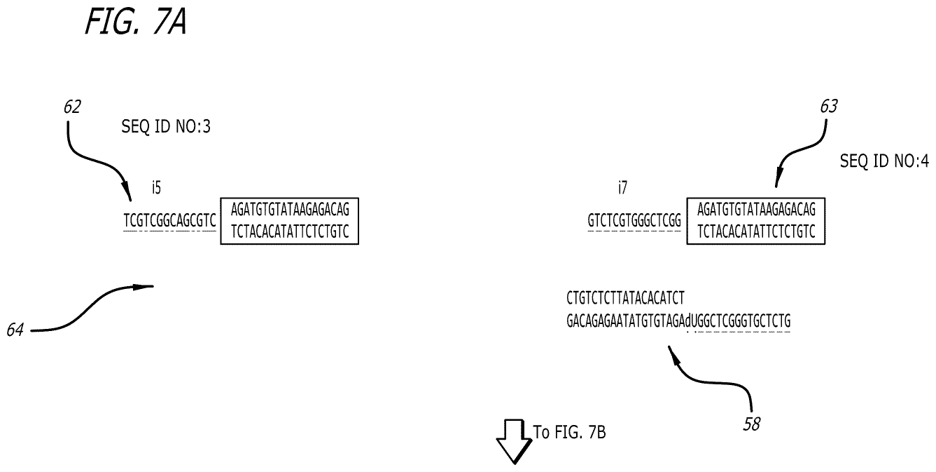

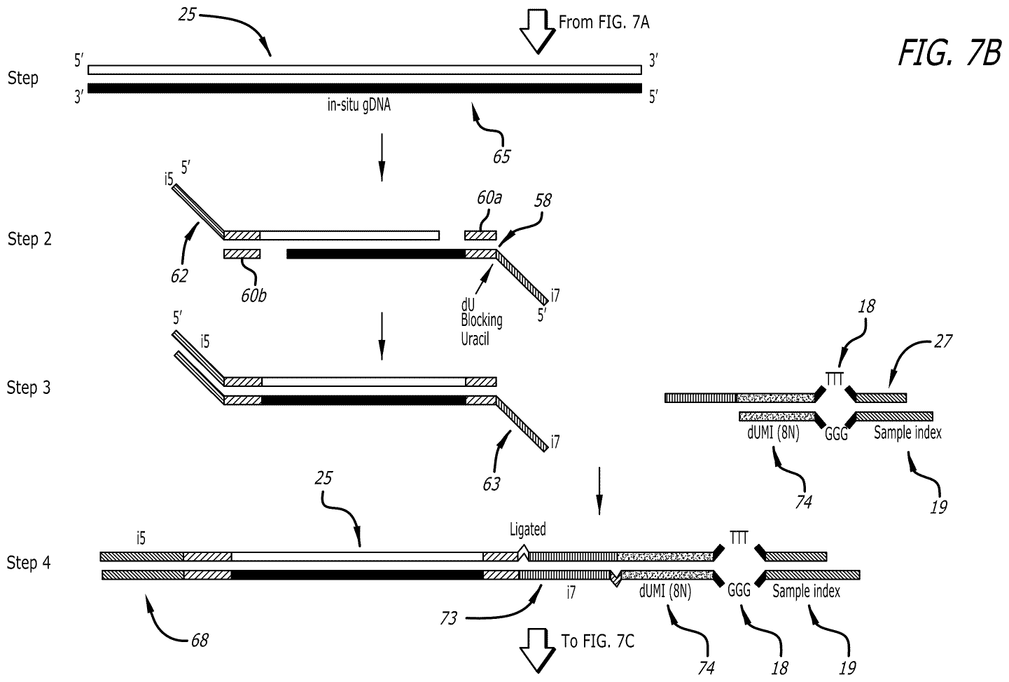

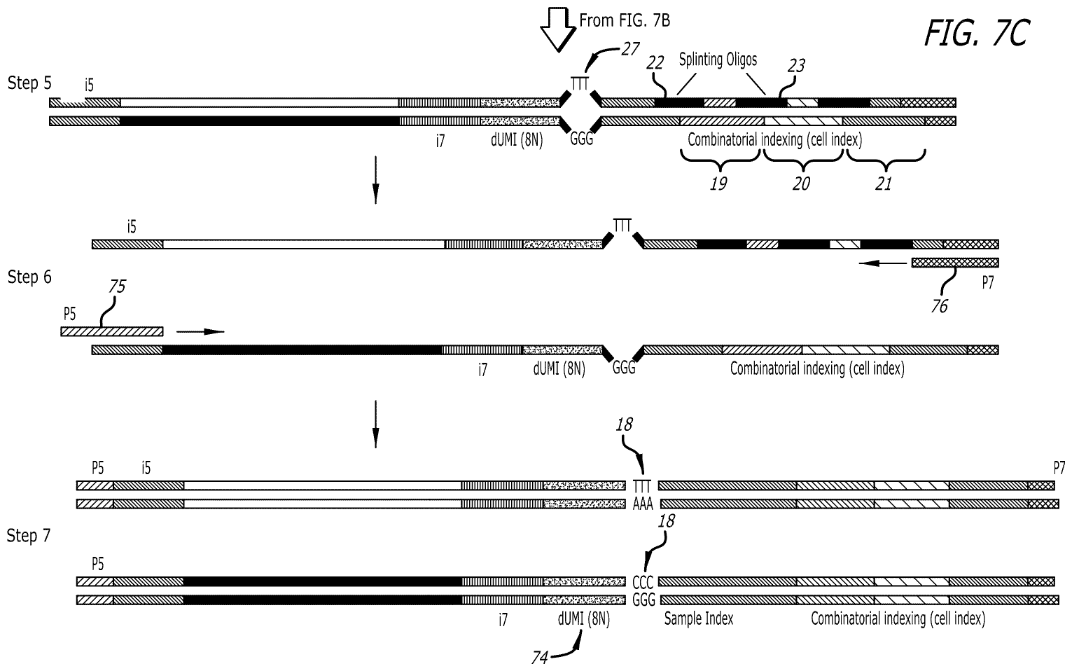

FIG. 7A illustrates nucleic acid sequencing adapter molecules for use with embodiments of the present technology. FIGS. 7B-7C are conceptual illustrations of various single-cell DS method steps utilizing a transposase, the sequencing adapters of FIG. 7A, and a combinatorial cell indexing scheme, and in accordance with another embodiment of the present technology. Newly synthesized strands denoted by text labels and/or brackets.

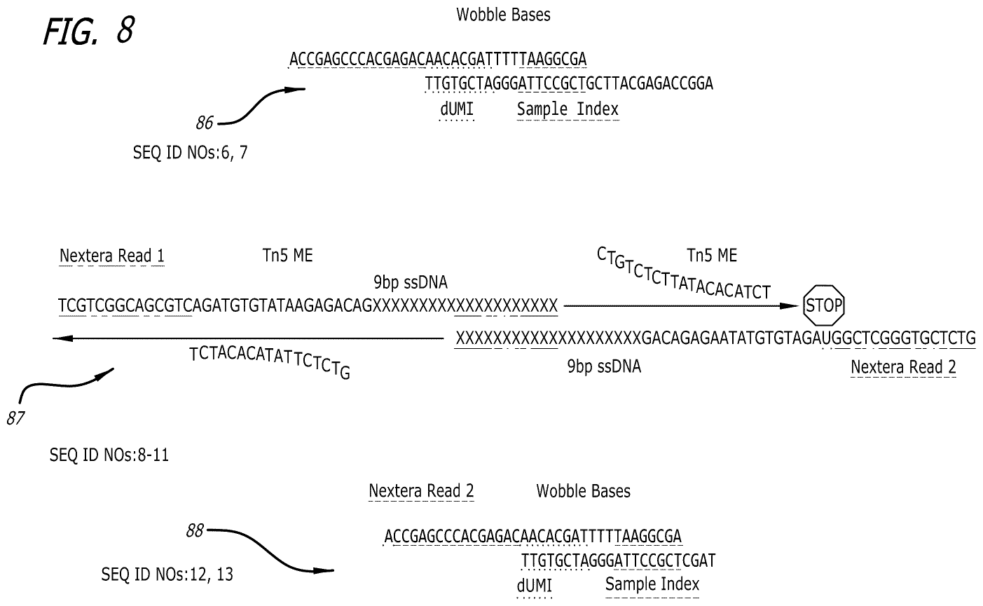

Referring now to FIGS. 7A, 7B, and 7C together, methods for preparing a sequencing library for duplex sequencing, utilizing adaptor ligation with a transposase, are presented. FIG. 8 is an illustration showing an example mechanistic detail for utilizing adaptor ligation with a transposase as shown at FIG. 7B, according to the disclosure.

As shown in FIG. 7A, sequencing adapters 64, comprised of adaptors 62 and 63, are included, and sequencing adaptor 58 (e.g., Illumina® i7 sequencing adaptor 1, wherein n=deoxyuracil (dU) (SEQ ID NOs.; 5, 10, 11)) can be used in the method illustrated in FIGS. 7B-7C. It will be recognized that while Illumina® compatible sequences and sequencing adapters are illustrated and discussed, one of ordinary skill in the art will recognize that the methods are suitable for preparing sequencing libraries for use on other NGS platforms (e.g., Element®, Singular®, MGI®, Ultima Genomics®, etc.).

Referring to FIGS. 7B and 7C together, fixed and permeabilized cells are provided for interrogation of cellular nucleic acid molecules and for preparing a sequencing library for single-cell DS. In Steps 1 and 2, cellular nucleic acid molecules 25 (e.g., in-situ gDNA 65 of FIG. 7B) are covalently attached to the adaptors 62, 58 via a transposase (e.g., Tn5). Integration of the adapter sequences occur at Tn5 transposition sites and according to ME elements 60a and 60b. FIG. 8 illustrates the mechanism of Tn5-adapter integration at Tn5 ME in accordance with embodiments. The deoxyuracil residue (dU) of adapter 58 blocks dU-intolerant polymerases, such that extension (Step 3) with a dU-intolerant polymerase results in a double-stranded i5 adaptor sequence and a single-stranded i7 adaptor sequence 63.

Referring to FIG. 7B, Steps 3 and 4, an indexing adapter 27 is provided with an overhang complementary to the single-stranded portion of i7 adapter sequence of 63. The indexing adapter 27 includes a double-stranded UMI 74, a mismatched base region that can function as a strand index 18 (e.g., an SDE), and a sample index barcode 19. In Step 4, the indexing adapter 27 is ligated to adapter-DNA complex (62, 25, 63) via ligation. The resultant molecule 68, e.g., gDNA ligated with a barcode containing a UMI 74, a base pair mismatch 18 for SDE, and a sample index 19, are ready for cell indexing.

Accordingly, in embodiments of such transposase methods, cell indexing is performed, as described previously herein, to form cell indices (FIG. 7C. Step 5; see FIGS. 3A-3D). As illustrated in FIG. 7C. Step 6, primers 75, 76 are annealed and primer extension via DNA polymerase is used to generate the NGS library (Step 7). Introduction of a platform-specific adaptor, e.g., . Illumina® adaptor (Index 2 or Index I adaptor; i7 or i5 adaptor; P5 and/or P7 as shown) enables interfacing of the final library with the sequencing platform.

As shown at FIG. 7C. Step 7, the NGS library provides distinct, yet related double-stranded molecules derived from the top and bottom strands of an original cellular nucleic acid molecule 25. Because both families contain the same sample and cell index (e.g., 19, 20, 21), the biological variants are traceable to individual cells.

Accordingly, in embodiments and as shown at FIG. 7C, nucleic acid molecules of the NGS library comprise a sample index (19), a cell index (19. 20. 21), a molecular index (UMI 74), and a strand index (e.g., 18). With duplex sequencing and data analysis, the sample index is for identification of a particular biological sample among biological samples, the cell index is for identification of a particular cell among cells, the molecular index is for identification of a particular cellular nucleic acid molecule among cellular nucleic acid molecules, and the strand index is for identification of a particular strand of the particular cellular nucleic acid molecule.

In the shown embodiment, the SDE 18 is introduced to the cellular nucleic acid molecules 25 by ligation with a corresponding aliquot-specific barcode nucleic acid 27 that comprises a base pair mismatch for the SDE 18 (e.g., “Wobble base”; see also FIG. 8). While the SDE is shown as being introduced in this example embodiment in this example manner, the SDE can be introduced with other methods without departing from the scope of the disclosure.

FIG. 9A-9B are conceptual illustrations of various duplex sequencing (DS) library preparation steps utilizing a transposase and in accordance with an embodiment of the present technology. In reference to the FIGS. 9A-9B the “IIIIIIII” refers to “Sample index”, “NNNNNNNN” refers to “UMI”, and (XXXXXXX″ refers to “Aliquot-specific barcode”. Newly synthesized strand elements are denoted in brackets.

Further aspects of the present technology include methods for preparing a duplex sequencing library from extracted DNA (e.g., ex vivo) using a transposase-mediated barcoding scheme. By way of example, FIGS. 9A-9B are conceptual illustrations of various duplex sequencing (DS) library preparation steps utilizing a transposase and in accordance with an embodiment of the present technology. In reference to the FIGS. 9A-9B the “NNNNNNNN” refers to “UMI”, and “XXXXXXXX” refers to “Sample-specific index” or other “barcode”. Newly synthesized strand elements are denoted with text labels and/or brackets.

With reference to FIGS. 9A and 9B together, a nucleic acid adapter molecule having a UMI 74 (UMI 74a, 74b) is integrated (e.g., bound, attached, ligated, etc.) with nucleic acid material (e.g., non-fragmented nucleic acid material) with a transposase. Following a transposition event (FIG. 9A, Step 1), the adapter molecules comprising UMIs 74a, 74b flank double-stranded nucleic acid fragments 25 (e.g., showing top strand 25a and bottom strand 25b) thereby forming tagged adapter-nucleic acid complexes. In the illustrated embodiment, integration of the adapter molecules occurs at transposition sites 28a and 28b and according to ME elements 60a and 60b (FIG. 9A, Step 1). As an example. FIG. 8 illustrates the mechanism of Tn5-adapter integration at Tn5 MEs in accordance with embodiments.

In the illustrated embodiment, the adapter molecules have 5′ overhang elements with one or more uracil bases (dUs) such that the original 5′ ends of each of the adapted first strand and adapted second strand include the element with uracil (Step 1). In Step 2, polymerase extension generates a double-stranded adapter-nucleic acid complexes wherein the copy strands do not have the uracil bases. In this embodiment, the polymerase is a uracil-tolerant polymerase. In this Step 2, polymerase extension allows for both original strands 25a, 25b to be associated with the UMIs 74a, 74b and the sample-specific index 19. In embodiments the non-uracil elements can be copied (e.g., with conical bases ATGC) to provide a platform specific adapter sequence (e.g., Illumina® adaptor (Index 2 or Index 1 adaptor; i7 or i5 adaptor sequences) or other amplification primer-binding site for library amplification and/or sequencing in subsequence method steps.

In FIG. 9A, Step 3, the top and bottom strands of the adapter-nucleic acid complexes are melted and SDE primers 40a and 40b are annealed (e.g., to non-uracil containing elements of the “copy” strands). In this embodiment, the SDE primers 40a. 40b comprise base pair mismatches (depicted as “CCC” in FIG. 9A, Step3). Polymerase extension from the SDE primers 40a, 40b generates a double-stranded nucleic acid molecule representing the original top strand and a double-stranded nucleic acid molecule representing the original bottom strand (FIG. 9A, Step 4). In this embodiment, the polymerase used in Step 4 is a uracil-tolerant polymerase. The resulting double-stranded adapter-nucleic acid molecules have SDE elements 18a and 18b. While the SDE elements associated with the derivatives of these top and bottom strands can be the same nucleotide sequence (e.g., “CCC”), the orientation of these elements (e.g., top strand has SDE 18a adjacent to the 3′ end of the nucleic acid molecule 25a, and bottom strand has SDE 18b adjacent to the 5′ end of the nucleic acid molecule 25b) provides the function of the strand index. In other embodiments, the SDE primers 40a and 40b can have different SDE elements (e.g., different from each other) so the different sequence of the base pair mismatch can, at least in part, provide the function of the strand index. While the SDE is shown as being introduced with use of the SDE primer 40, the SDE can be introduced with other methods without departing from the scope of the disclosure.

Referring to FIG. 9A, Step 5, the resulting double-stranded adapter-nucleic acid molecules are enzymatically treated to remove the uracil-containing elements from the original adapter-nucleic acid strands. In one example, the adapter-nucleic acid molecules can be treated with uracil DNA glycosylase (UDG) or a mixture of uracil DNA glycosylase and a single-strand endonuclease, such as endonuclease III or endonuclease VIII or endonuclease IV (e.g., also commercially available as USERR II Enzyme (New England Biolabs, M5508S or M5508L)) to destroy the element, thereby preventing subsequence primer binding. In embodiments, use of and subsequent removal of the uracil-containing elements allows for filtering or removing from processing of the original nucleic acid strands that do not yet have an SDE. Alternatively, sequence reads not having an SDE can be removed during data analysis.

With reference to FIG. 9B. Step 6, the method includes providing primers 41a and 41b which anneal to the 3′ ends (e.g., non-uracil containing elements) of the “copy” strands. As shown, primers 41a and 41b include primer tails providing platform-specific adapter sequences (e.g., IlluminaR adaptor (Index 2 or Index 1 adaptor; i7 or i5 adaptor sequences). With reference to Steps 6 and 7, polymerase extension from annealed primers 41a and 41b provide double stranded adapter-nucleic acid molecules wherein each nucleic acid fragment 25a. 25b has flanking adapter regions comprising mosaic end sequences 60. UMI sequences 74a, 74b, sample index barcodes 19, strand index as SDE 18a. 18b, and primer binding sites (for further exponential amplification) and/or flow cell binding sites 84. 85 (FIG. 9B. Steps 6 and 7). The resultant sequencing library as prepared through transposon-mediated fragmentation and adapter integration provides a sample index (for differentiating nucleic acids within multiplexed samples), molecular indexes (for differentiating molecules from other molecules in a sample), and a strand index for differentiating sequencing information derived from each of the two strands to facilitate sequence comparison from the two strands of an original molecule for duplex sequence error correction. In embodiments not shown, the above transposon-mediated tagging scheme can be used without the use of an additional UMI sequence (e.g., 74a, 74b). In such embodiments, information related to fragment ends, mosaic elements (ME) or a combination thereof can be used as a molecular index.

### Unique Molecule Identifier Sequences (UMIs)

In accordance with various embodiments, provided methods and compositions include one or more UMI sequences on each strand of a double-stranded nucleic acid material. The UMI can be independently carried by each of the single strands that result from a double-stranded nucleic acid molecule such that the derivative amplification products of each strand can be recognized as having come from the same original substantially unique double-stranded nucleic acid molecule after sequencing. In embodiments, the UMI may include additional information and/or may be used in other methods for which such molecule distinguishing functionality is useful, as will be recognized by one of skill in the art. In embodiments, a UMI element may be incorporated before, substantially simultaneously, or after cell index integration steps (e.g., adapter ligation, adapter tagmentation) to a nucleic acid material.

In embodiments, a UMI sequence may include at least one degenerate or semi degenerate nucleic acid. In other embodiments, a UMI sequence may be non-degenerate. In embodiments, the UMI can be the sequence associated with or near a fragment end of the nucleic acid molecule (e.g., randomly or semi-randomly fragmented ends of indexed nucleic acid material). In embodiments, an exogenous sequence may be considered in conjunction with the sequence corresponding to randomly or semi-randomly fragmented ends of indexed nucleic acid material (e.g., DNA) to obtain a UMI sequence capable of distinguishing, for example, individual DNA molecules from one another. In embodiments, a UMI sequence is a portion of an adapter sequence that is ligated to a double-strand nucleic acid molecule. In other embodiments, the adapter sequencing comprising a UMI is incorporated with double-stranded nucleic acid material by transposase tagmentation. In certain embodiments, the adapter sequence comprising a UMI sequence is double-stranded such that each strand of the double-stranded nucleic acid molecule includes a UMI following ligation or tagmentation to the adapter sequence. In another embodiment, the UMI sequence is single-stranded before or after ligation to a double-stranded nucleic acid molecule and a complimentary UMI sequence can be generated by extending the opposite strand with a DNA polymerase to yield a complementary double-stranded UMI sequence. In other embodiments, a UMI sequence is in a single-stranded portion of the adapter (e.g., an arm of an adapter having a Y-shape). In such embodiments, the UMI can facilitate grouping of families of sequence reads derived from an original strand of a double-stranded nucleic acid molecule, and in some instances can confer relationship between original first and second strands of a double-stranded nucleic acid molecule (e.g., all or part of the UMIs maybe relatable via look up table). In embodiments, where the first and second strands are labeled with different UMIs, the sequence reads from the two original strands may be related using one or more of an endogenous UMI (e.g., a fragment-specific feature such as sequence associated with or near a fragment end of the nucleic acid molecule), or with use of an additional molecular tag shared by the two original strands (e.g., a barcode in a double-stranded portion of the adapter), or a combination thereof. In embodiments, each UMI sequence may include between about 1 to about nucleic acids (e.g., 1, 2, 3, 4. 5. 8, 10, 12. 14. 16. 18. 20, or more degenerate or semi-degenerate nucleic acids).

In embodiments, a UMI is capable of being ligated to one or both of a nucleic acid material and an adapter sequence. In embodiments, a UMI may be ligated to at least one of a T-overhang, an A-overhang, a CG-overhang, a dehydroxylated base, and a blunt end of a nucleic acid material. In embodiments, a UMI sequence may be added via primer extension or combinatorial cell indexing (e.g., during sequencing library preparation) as described herein. In other embodiments, the UMI may comprise all or portion of mosaic elements.

In embodiments, a sequence of a UMI may be considered in conjunction with (or designed in accordance with) the sequence corresponding to, for example, randomly or semi randomly fragmented ends of a nucleic acid material (e.g., a ligated nucleic acid material, fragment ends generated during tagmentation, etc.), to obtain a UMI sequence capable of distinguishing individual nucleic acid molecules from one another.

In embodiments, at least one UMI may be an endogenous UMI (e.g., a UMI related to a fragment end region (e.g., a fragment end, a sheared point, reference sequence mapping coordinates, etc.), for example, using the fragment end itself or using a defined number of nucleotides in the nucleic acid material immediately adjacent to the fragment end (e.g., 2, 3, 4. 5, 6, 7, 8, 9. 10 nucleotides from the fragment end)). In embodiments, at least one UMI may be an exogenous UMI sequence (e.g., a UMI comprising a sequence that is not found on a target nucleic acid material).

In embodiments, a UMI may be or comprise an imaging moiety (e.g., a fluorescent or otherwise optically detectable moiety). In embodiments, such UMIs allow for detection and/or quantitation without the need for an amplification step.

In various embodiments, the UMI sequence may be a combination of sequences that together form the UMI. Such UMI sequences may be adjacent to one other or separated from one another in any particular molecule within a prepared sequence library. In embodiments a UMI element may comprise two or more distinct UMI elements that are located at different locations on the indexed-target nucleic acid complex.

### Strand-Defining Element (SDE)

In embodiments, each strand of a double-stranded nucleic acid material may further include an element that renders the amplification products of the two single-stranded nucleic acids that form the target double-stranded nucleic acid material substantially distinguishable from each other after sequencing. In embodiments, an SDE may be or comprise a base-pair mismatch (e.g., wobble base(s)). In embodiments, each base-pair mismatch may include between about 1 to about mismatched nucleic acids (e.g., 1, 2, 3, 4, 5, 8, 10, 12, 14, 16, 18, 20). In embodiments, each base-pair mismatch may include between about 2 and about 10 bases (e.g., about 2, 3, 4, 5, 6, 7, 8, 9, or about 10 bases).

In embodiments, a SDE may be or comprise asymmetric primer sites comprised within a sequencing adapter, or, in other arrangements, sequence asymmetries may be introduced into the adapter sequences and not within the primer sequences, such that at least one position in the nucleotide sequences of a first strand target nucleic acid sequence complex and a second stand of the target nucleic acid sequence complex are different from each other following amplification and sequencing. In other embodiments, the SDE may comprise another biochemical asymmetry between the two strands that differs from the canonical nucleotide sequences A. T. C. G or U, but is converted into at least one canonical nucleotide sequence difference in the two amplified and sequenced molecules. In yet another embodiment, the SDE may be or comprise a means of physically separating the two strands before amplification, such that derivative amplification products from the first strand target nucleic acid sequence and the second strand target nucleic acid sequence are maintained in substantial physical isolation from one another for the purposes of maintaining a distinction between the two derivative amplification products. Other such arrangements or methodologies for providing an SDE function that allows for distinguishing the first and second strands may be utilized.

In embodiments, a SDE may be capable of forming a loop (e.g., a hairpin loop). In embodiments, a loop may comprise at least one endonuclease recognition site. In embodiments, the target nucleic acid complex may contain an endonuclease recognition site that facilitates a cleavage event within the loop. In embodiments, a loop may comprise a non-canonical nucleotide sequence. In embodiments, the contained non-canonical nucleotide may be recognizable by one or more enzyme that facilitates strand cleavage. In embodiments, the contained non-canonical nucleotide may be targeted by one or more chemical process facilitates strand cleavage in the loop. In embodiments the loop may contain a modified nucleic acid linker that may be targeted by one or more enzymatic, chemical or physical process that facilitates strand cleavage in the loop. In embodiments this modified linker is a photocleavable linker.

A variety of other molecular tools could serve as UMIs and SDEs. Other than shear points and DNA-based tags, single-molecule compartmentalization methods that keep paired strands in physical proximity or other non-nucleic acid tagging methods could serve the strand-relating function. Similarly, asymmetric chemical labelling of the adapter strands in a way that they can be physically separated can serve an SDE role. A recently described variation of Duplex Sequencing uses bisulfite conversion to transform naturally occurring strand asymmetries in the form of cytosine methylation into sequence differences that distinguish the two strands. Although this implementation limits the types of mutations that can be detected, the concept of capitalizing on native asymmetry is noteworthy in the context of emerging sequencing technologies that can directly detect modified nucleotides. Various embodiments of SDEs are further disclosed in International Patent Publication No. WO2017/100441, which is incorporated by reference in its entirety.

### Adapters and Adapter Sequences

In various arrangements, adapter molecules that comprise UMIs (e.g., molecular barcodes), SDEs, primer sites, flow cell sequences and/or other features are contemplated for use with many of the embodiments disclosed herein. In embodiments, provided adapters may be or comprise one or more sequences complimentary or at least partially complimentary to PCR primers (e.g., primer sites) that have at least one of the following properties; 1) high target specificity; 2) capable of being multiplexed; and 3) exhibit robust and minimally biased amplification.

In embodiments, adapter molecules can be “Y”-shaped. “U”-shaped, “hairpin” shaped, have a bubble (e.g., a portion of sequence that is non-complimentary), be single-stranded, be double-stranded, be partially double-stranded or have other features. In other embodiments, adapter molecules can comprise a “Y”-shape, a “U”-shape, a “hairpin” shape, or a bubble. Certain adapters may comprise modified or non-standard nucleotides, restriction sites, or other features for manipulation of structure or function in vitro. Adapter molecules may ligate to a variety of nucleic acid material having a terminal end. For example, adapter molecules can be suited to ligate to a T-overhang, an A-overhang, a CG-overhang, a multiple nucleotide overhang, a dehydroxylated base, a blunt end of a nucleic acid material and the end of a molecule where the 5′ of the target is dephosphorylated or otherwise blocked from traditional ligation. In other embodiments the adapter molecule can contain a dephosphorylated or otherwise ligation-preventing modification on the 5′ strand at the ligation site. In the latter two embodiments, such strategies may be useful for preventing dimerization of library fragments or adapter molecules.

An adapter sequence can mean a single-strand sequence, a double-strand sequence, a complimentary sequence, a non-complimentary sequence, a partial complimentary sequence, an asymmetric sequence, a primer binding sequence, a flow-cell sequence, a ligation sequence, or other sequence provided by an adapter molecule. In particular embodiments, an adapter sequence can mean a sequence used for amplification by way of compliment to an oligonucleotide.

In embodiments, provided methods and compositions include at least one adapter sequence (e.g., two adapter sequences, one on each of the 5′ and 3′ ends of a nucleic acid material). In embodiments, provided methods and compositions may comprise 2 or more adapter sequences (e.g., 3. 4, 5, 6, 7, 8, 9, 10 or more). In embodiments, at least two of the adapter sequences differ from one another (e.g., by sequence). In embodiments, each adapter sequence differs from each other adapter sequence (e.g., by sequence). In embodiments, at least one adapter sequence is at least partially non-complementary to at least a portion of at least one other adapter sequence (e.g., is non-complementary by at least one nucleotide).

In embodiments, an adapter sequence comprises at least one non-standard nucleotide. In embodiments, a non-standard nucleotide is selected from an abasic site, a uracil. tetrahydrofuran, 8-oxo-7,8-dihydro-2′deoxyadenosine (8-oxo-A). 8-oxo-7,8-dihydro-2′-deoxyguanosine (8-oxo-G), deoxyinosine. 5′nitroindole, 5-Hydroxymethyl-2′-deoxycytidine, iso cytosine. 5 ‘-methyl-isocytosine, or isoguanosine, a methylated nucleotide, an RNA nucleotide, a ribose nucleotide, an 8-oxo-guanine, a photocleavable linker, a biotinylated nucleotide, a desthiobiotin nucleotide, a thiol modified nucleotide, an acrydite modified nucleotide an iso-dC. an iso dG, a 2’-O-methyl nucleotide, an inosine nucleotide Locked Nucleic Acid, a peptide nucleic acid, a 5 methyl dC, a 5-bromo deoxyuridine, a 2,6-Diaminopurine. 2-Aminopurine nucleotide, an abasic nucleotide, a 5-Nitroindole nucleotide, an adenylated nucleotide, an azide nucleotide, a digoxigenin nucleotide, an I-linker, an 5′ Hexynyl modified nucleotide, an 5-Octadiynyl dU. photocleavable spacer, a non-photocleavable spacer, a click chemistry compatible modified nucleotide, and any combination thereof.

In embodiments, an adapter sequence comprises a moiety having a magnetic property (i.e., a magnetic moiety). In embodiments this magnetic property is paramagnetic. In embodiments where an adapter sequence comprises a magnetic moiety (e.g., a nucleic acid material ligated to an adapter sequence comprising a magnetic moiety), when a magnetic field is applied, an adapter sequence comprising a magnetic moiety is substantially separated from adapter sequences that do not comprise a magnetic moiety (e.g., a nucleic acid material ligated to an adapter sequence that does not comprise a magnetic moiety).

### Primers

In embodiments, one or more PCR primers that have at least one of the following properties; 1) high target specificity; 2) capable of being multiplexed; and 3) exhibit robust and minimally biased amplification are contemplated for use in various embodiments in accordance with aspects of the present technology. A number of prior studies and commercial products have designed primer mixtures satisfying a certain number of these criteria for conventional PCR. However, it has been noted that these primer mixtures are not always optimal for use with MPS. Indeed, developing highly multiplexed primer mixtures can be a challenging and time-consuming process. Conveniently, both IlluminaR and PromegaR have recently developed multiplex compatible primer mixtures for the Illumina® platform that show robust and efficient amplification of a variety of standard and non-standard STR and SNP loci. Because these kits use PCR to amplify their target regions prior to sequencing, the 5′-end of each read in paired-end sequencing data corresponds to the 5′-end of the PCR primers used to amplify the DNA. In embodiments, provided methods and compositions include primers designed to ensure uniform amplification, which may entail varying reaction concentrations, melting temperatures, and minimizing secondary structure and intra/inter-primer interactions. Many techniques have been described for highly multiplexed primer optimization for MPS applications. In particular, these techniques are often known as ampliseq methods, as well described in the art.

### Amplification

Provided methods and compositions, in various embodiments, make use of, or are of use in, at least one amplification step wherein a nucleic acid material (or portion thereof, for example, a specific target region or locus) is amplified to form an amplified nucleic acid material (e.g., some number of amplicon products).

In embodiments, amplifying a nucleic acid material includes a step of amplifying nucleic acid material derived from each of a first and second nucleic acid strand from an original double-stranded nucleic acid material using at least one single-stranded oligonucleotide at least partially complementary to a sequence present in a first adapter sequence. An amplification step further includes employing a second single-stranded oligonucleotide to amplify each strand of interest, and such second single-stranded oligonucleotide can be (a) at least partially complementary to a target sequence of interest, or (b) at least partially complementary to a sequence present in a second adapter sequence such that the at least one single-stranded oligonucleotide and a second single-stranded oligonucleotide are oriented in a manner to effectively amplify the nucleic acid material.

In embodiments, amplifying nucleic acid material in a sample can include amplifying nucleic acid material in “tubes” (e.g., PCR tubes), in wells (e.g., of multi-well plate), in emulsion droplets, microchambers, and other examples described above or other known vessels.

In embodiments, at least one amplifying step includes at least one primer that is or comprises at least one non-standard nucleotide. In embodiments, a non-standard nucleotide is selected from a uracil, a methylated nucleotide, an RNA nucleotide, a ribose nucleotide, an 8-oxo-guanine, a biotinylated nucleotide, a locked nucleic acid, a peptide nucleic acid, a high-Tm nucleic acid variant, an allele discriminating nucleic acid variant, any other nucleotide or linker variant described elsewhere herein and any combination thereof.

While any application-appropriate amplification reaction is contemplated as compatible with embodiments, by way of specific example, in embodiments, an amplification step may be or comprise a polymerase chain reaction (PCR), rolling circle amplification (RCA), multiple displacement amplification (MDA), isothermal amplification, polony amplification within an emulsion, bridge amplification on a surface, the surface of a bead or within a hydrogel, and any combination thereof.

In embodiments, amplifying a nucleic acid material includes use of single-stranded oligonucleotides at least partially complementary to regions of the adapter sequences on the 5′ and 3′ ends of each strand of the nucleic acid material. In embodiments, amplifying a nucleic acid material includes use of at least one single-stranded oligonucleotide at least partially complementary to a target region or a target sequence of interest (e.g., a genomic sequence, a mitochondrial sequence, a plasmid sequence, a synthetically produced target nucleic acid, etc.) and a single-stranded oligonucleotide at least partially complementary to a region of the adapter sequence (e.g., a primer site).

In general, robust amplification (for example PCR amplification), can be highly dependent on the reaction conditions. Multiplex PCR, for example, can be sensitive to buffer composition, monovalent or divalent cation concentration, detergent concentration, crowding agent (i.e. PEG, glycerol, etc.) concentration, primer concentrations, primer Tms, primer designs, primer GC content, primer modified nucleotide properties, and cycling conditions (i.e. temperature and extension times and rate of temperature changes). Optimization of buffer conditions can be a difficult and time-consuming process. In embodiments, an amplification reaction may use at least one of a buffer, primer pool concentration, and PCR conditions in accordance with a previously known amplification protocol. In embodiments, a new amplification protocol may be created, and/or an amplification reaction optimization may be used. By way of specific example, in embodiments, a PCR optimization kit may be used, such as a PCR Optimization Kit from PromegaR. which contains a number of pre-formulated buffers that are partially optimized for a variety of PCR applications, such as multiplex, real-time. GC-rich, and inhibitor-resistant amplifications. These pre-formulated buffers can be rapidly supplemented with different Mg²⁺ and primer concentrations, as well as primer pool ratios. In addition, in embodiments, a variety of cycling conditions (e.g., thermal cycling) may be assessed and/or used. In assessing whether or not a particular embodiment is appropriate for a particular desired application, one or more of specificity, allele coverage ratio for heterozygous loci, interlocus balance, and depth, among other aspects, may be assessed. Measurements of amplification success may include DNA sequencing of the products, evaluation of products by gel or capillary electrophoresis or HPLC or other size separation methods followed by fragment visualization, melt curve analysis using double-stranded nucleic acid binding dyes or fluorescent probes, mass spectrometry or other methods known in the art.

In accordance with various embodiments, any of a variety of factors may influence the length of a particular amplification step (e.g., the number of cycles in a PCR reaction, etc.). For example, in embodiments, a provided nucleic acid material may be compromised or otherwise suboptimal (e.g., degraded and/or contaminated). In such a case, a longer amplification step may be helpful in ensuring a desired product is amplified to an acceptable degree. In embodiments, an amplification step may provide an average of 3 to 10 sequenced PCR copies from each starting DNA molecule, though in other embodiments, only a single copy of each of a first strand and second strand are required. Without wishing to be held to a particular theory, it is possible that too many or too few PCR copies could result in reduced assay efficiency and, ultimately, reduced depth. Generally, the number of nucleic acid (e.g., DNA) fragments used in an amplification (e.g., . PCR) reaction is a primary adjustable variable that can dictate the number of reads that share the same UMI/barcode sequence.

### Nucleic Acid Material Types

In accordance with various embodiments, any of a variety of nucleic acid material may be targeted in a single-cell DS method. In embodiments, the nucleic acid material is or comprises at least one of double-stranded DNA, single-stranded DNA, double-stranded RNA, single-stranded RNA. In embodiments, the nucleic acid material is or comprises gDNA, mitochondrial DNA, and/or RNA, such as messenger RNA (mRNA) (e.g., RNA transcripts). In accordance with aspects of the present technology, the nucleic acid material can be naturally-occurring cellular nucleic acid material.

### Modifications

In accordance with various embodiments, nucleic acid material may receive one or more modifications prior to, substantially simultaneously, or subsequent to, any particular step. depending upon the application for which a particular provided method or composition is used.

In embodiments, a modification may be or comprise repair of at least a portion of the nucleic acid material. While any application-appropriate manner of nucleic acid repair is contemplated as compatible with embodiments, certain exemplary methods and compositions therefore are described below and in the Examples.

By way of non-limiting example, in embodiments, DNA repair enzymes, such as Uracil-DNA Glycosylase (UDG), Formamidopyrimidine DNA glycosylase (FPG), and 8-oxoguanine DNA glycosylase (OGGI), can be utilized to correct DNA damage. As discussed above, these DNA repair enzymes, for example, are glycoslyases that remove damaged bases from DNA. For example, UDG removes uracil that results from cytosine deamination (caused by spontaneous hydrolysis of cytosine) and FPG removes 8-oxo-guanine (e.g., most common DNA lesion that results from reactive oxygen species). FPG also has lyase activity that can generate 1 base gap at abasic sites. Such abasic sites will subsequently fail to amplify by PCR, for example, because the polymerase fails copy the template. Accordingly, the use of such DNA damage repair enzymes can effectively remove damaged DNA that doesn't have a true mutation but might otherwise be undetected as an error following sequencing and duplex sequence analysis.

### Targeted Enrichment

In accordance with various embodiments, sequencing libraries may be prepared for whole genome. whole transcriptome, whole exome, or for targeted panels of nucleic acid material, wherein targeted regions of the genome or transcriptome may be enriched for particular regions of the genome. Any known ways of enriching target nucleic acids for NGS library preparation may be used, including but not limited to hybridization-based targeted capture, or in another embodiment. with multiplex PCR using primer(s) specific for an adapter sequence and primer(s) specific to the target nucleic acid region(s) of interest (e.g., . Split-DS. U.S. Pat. No. 11,479,807. incorporated herein by reference in its entirety).

### Sequencing Platforms

It will be recognized that while IlluminaR compatible sequences and sequencing adapters are illustrated and discussed, one of ordinary skill in the art will recognize that the methods are suitable for preparing sequencing libraries for use on other NGS platforms (e.g., ElementR. SingularR. MGIR. Ultima GenomicsR, etc.).

Kits with Reagents

Aspects of the present technology further encompass kits for conducting various aspects of single cell DS methods (also referred to herein as a “scDS kit”). In embodiments, a kit may comprise various reagents along with instructions for conducting one or more of the methods or method steps disclosed herein for single cell fixation, permeabilization, nucleosome removal, double-strand DNA fragmentation (e.g., in vivo), sample and cell indexing, molecular indexing. nucleic acid extraction, and further steps of nucleic acid library preparation, amplification (e.g., via PCR) and sequencing. In embodiments, a kit may further include a computer program product (e.g., coded algorithm to run on a computer, an access code to a cloud-based server for running one or more algorithms, etc.) for analyzing sequencing data (e.g., raw sequencing data, sequencing reads, etc.) to identify, for example, a sequence variant or mutation associated with a specific cell type or a particular cell, a combination of variants associated with a specific cell type or a particular cell, a disease-associated mutation or a disease state of a subject, a cancer, a cancer risk, etc, associated with a sample and in accordance with aspects of the present technology. Kits may include DNA standards and other forms of positive and negative controls.

In embodiments, a scDS kit may comprise reagents or combinations of reagents suitable for performing various aspects of sample preparation (e.g., tissue manipulation, cell separation, cell fixation, nuclei isolation, cell and organelle permeabilization, DNA fragmentation, sample and cell indexing and other molecule labeling steps, combinatorial cell indexing, tagmentation of DNA, adapter and oligo design. DNA extraction. DNA purification. RNA reverse transcription. cDNA purification, etc.), other nucleic acid library preparation, amplification and sequencing. For example, a scDS kit may optionally comprise one or more DNA extraction reagents (e.g., buffers, columns, etc.) and/or tissue extraction reagents. Optionally, a scDS kit may further comprise one or more reagents or tools for fragmenting double-stranded DNA, such as by enzymatic means (e.g., enzymes for random or semi-random genomic shearing and appropriate reaction enzymes). For example, a kit may include DNA fragmentation reagents for enzymatically fragmenting double-stranded DNA that includes one or more of enzymes for targeted digestion (e.g., restriction endonucleases. CRISPR/Cas endonuclease(s) and RNA guides, and/or other endonucleases), double-stranded Fragmentase cocktails, single-stranded DNase enzymes (e.g., mung bean nuclease, Sl nuclease) for rendering fragments of DNA predominantly double-stranded and/or destroying single-stranded DNA, and appropriate buffers and solutions to facilitate such enzy matic reactions.

In other embodiments, scDS kits may include DNA transposase and tagmentation reagents for preparing sequence libraries suitable for single cell DS. For example, a scDS kit may include one or more transposase enzymes and tagmentation reaction buffer(s).

In embodiments, a scDS kit comprises reagents for preparing cells for in vivo indexing of cellular nucleic acid material. For example, the kit may include reagents for cell fixation (e.g., formaldehyde in phosphate buffered saline (PBS), methanol, ethanol, acetic acid, acetone or other solutions). The kit may also include reagents for permeabilization of cells and/or organelles (e.g., nuclei, mitochondria) such as, e.g., . TRITON™ X-100, HCl, ethanol, methanol. Tween 20, and/or NP-40, and/or other solutions. In other embodiments, the kit may include reagents for nucleosome depletion (e.g., sodium dodecyl sulfate (SDS), lithium diiodosalicylate).

In embodiments, the scDS kit comprises enzymes and reagent for reverse transcribing RNA molecules to provide cDNA molecules. For example, the kit may include a reverse transcriptase, and a reverse transcription primer that can be designed to (a) reverse transcribe all, or substantially all, RNA in a cell (e.g., a random hexamer with a 5′ overhang). (b) reverse transcribe RNA having a poly (A) tail (e.g., a poly (dT) primer, such as a dI (15) primer, with a 5′ overhang), or (c) reverse transcribe predetermined RNAs (e.g., a transcript-specific primer).

In an embodiment, a scDS kit comprises primers and adapters for preparing a nucleic acid sequence library from a sample that is suitable for performing single cell Duplex Sequencing process steps to generate error-corrected (e.g., high accuracy) sequences of double-stranded nucleic acid molecules in the cells. For example, the kit may comprise at least one pool or mix of adapter molecules and/or oligonucleotides comprising sample index barcodes. UMI sequences. cell indexing barcodes, base pair mismatches, uracil or other non-conical bases, etc., or the tools (e.g., single-stranded oligonucleotides) for the user to create it. In embodiments, the pool of adapter molecules will comprise a suitable number of substantially unique UMI sequences such that a plurality of nucleic acid molecules in a cell can be substantially uniquely labeled following attachment of the adapter molecules, either alone or in combination with unique features of the fragments to which they are attached. One experienced in the art of molecular tagging will recognize that what entails a “suitable” number of UMI sequences will vary by multiple orders of magnitude depending on various specific factors (input DNA, type of DNA fragmentation, average size of fragments, complexity vs repetitiveness of sequences being sequenced within a genome etc.). Optionally, the adaptor molecules and/or oligonucleotides further include one or more PCR primer binding sites, one or more sequencing primer binding sites, or both. In another embodiment, a scDS kit does not include adapter molecules comprising UMI sequences or barcodes, but instead includes conventional sequencing adapter molecules (e.g., platform-specific adaptor sequence, e.g., Illumina® adaptor (Index 2 or Index 1 adaptor; 17 or i5 adaptor), etc.) and various method steps can utilize endogenous UMIs to relate molecule sequence reads. In embodiments, the adapter molecules are indexing adapters and/or comprise an indexing sequence. In other embodiments, indexes are added to specific samples through “tailing in” by PCR using primers supplied in a kit.

Additionally, a kit may further include DNA quantification materials such as, for example, DNA binding dye such as SYBRIM green or SYBRIM gold (available from Thermo Fisher ScientificR′. Waltham, MA) or the alike for use with a Qubit fluorometer (e.g., available from Thermo Fisher ScientificR, Waltham, MA), or PicoGreen™ dye (e.g., available from Thermo Fisher ScientificR, Waltham, MA) for use on a suitable fluorescence spectrometer or a real-time PCR machine or digital-droplet PCR machine. Other reagents suitable for DNA quantification on other platforms are also contemplated. Further embodiments include kits comprising one or more of nucleic acid size selection reagents (e.g., Solid Phase Reversible Immobilization (SPRI) magnetic beads, gels, columns), columns for target DNA capture using bait/pray hybridization, qPCR reagents (e.g., for copy number determination) and/or digital droplet PCR reagents. In embodiments, a kit may optionally include one or more of library preparation enzymes (ligase, polymerase(s), endonuclease(s), transposase(s), reverse transcriptase for e.g., RNA interrogations), dNTPs, buffers, capture reagents (e.g., beads, surfaces, coated tubes. columns, etc.), indexing primers, amplification primers (PCR primers) and sequencing primers. In embodiments, a kit may include reagents for assessing types of DNA damage such as an error-prone DNA polymerase and/or a high-fidelity DNA polymerase. Additional additives and reagents are contemplated for PCR or ligation reactions in specific conditions (e.g., high GC rich genome/target).

In an embodiment, the kits further comprise reagents, such as DNA error correcting enzymes that repair DNA sequence errors that interfere with polymerase chain reaction (PCR) processes (versus repairing mutations leading to disease). By way of non-limiting example, the enzymes comprise one or more of the following; monofunctional uracil-DNA glycosylase (hSMUGI). Uracil-DNA Glycosylase (UDG). N-glycosylase/AP-Ivase NEIL 1 protein (hNEILI), Formamidopyrimidine DNA glycosylase (FPG), 8-oxoguanine DNA glycosylase (OGG 1). human apurinic/apyrimidinic endonuclease (APE 1), endonuclease III (Endo III), endonuclease IV (Endo IV), endonuclease V (Endo V), endonuclease VIII (Endo VIII), T7 endonuclease I (T7 Endo I), T4 pyrimidine dimer glycosylase (T4 PDG), human single-strand-selective human alkyladenine DNA glycosylase (hAAG), etc., among other glycosylases. Iyases, endonucleases and exonucleases etc.; and can be utilized to correct DNA damage (e.g., in vitro or in vivo DNA damage). Some of such DNA repair enzymes, for example, are glycoslyases that remove damaged bases from DNA. For example, UDG removes uracil that results from cytosine deamination (caused by spontaneous hydrolysis of cytosine) and FPG removes 8-oxo-guanine (e.g., most common DNA lesion that results from reactive oxygen species). FPG also has lyase activity that can generate 1 base gap at abasic sites. Such abasic sites will subsequently fail to amplify by PCR, for example, because the polymerase fails copy the template. Accordingly, the use of such DNA damage repair enzymes, and/or others listed here and as known in the art, can effectively remove damaged DNA that does not have a true mutation but might otherwise be undetected as an error.

In other embodiments where uracil containing elements are present and desirable to remove as part of the sequencing library preparation steps, the scDS kit may include using a uracil DNA glycosylase (UDG) or a mixture of uracil DNA glycosy lase and single-strand endonuclease, such as endonuclease III or endonuclease VIII or endonuclease IV (e.g., USERR II Enzyme (New England Biolabs®, M5508S or M5508L)).

The kits may further comprise appropriate controls, such as DNA amplification controls, nucleic acid (template) quantification controls, sequencing controls, nucleic acid molecules derived from a reference biological source. In embodiments, a kit may include a control population of cells. In an embodiment, the kit comprises containers for shipping samples, storage material for stabilizing samples, material for freezing samples, such as cell samples, for analysis to detect DNA variants in a subject sample. In another embodiment, a kit may include nucleic acid contamination control standards (e.g., hybridization capture probes or gene specific amplification primers with affinity to genomic regions in an organism that is different than the test or subject organism).

The kit may further comprise one or more other containers comprising materials desirable from a commercial and user standpoint, including PCR and sequencing buffers, diluents, subject sample extraction tools (e.g., syringes, swabs, etc.), and package inserts with instructions for use. In addition, a label can be provided on the container with directions for use, such as those described above; and/or the directions and/or other information can also be included on an insert which is included with the kit; and/or via a website address provided therein. The kit may also comprise laboratory tools such as, for example, sample tubes, plate sealers, microcentrifuge tube openers, labels, magnetic particle separator, foam inserts, ice packs, dry ice packs, insulation, etc.

The kits may further comprise a computer program product installable on an electronic computing device (e.g., laptop/desktop computer, tablet, etc.) or accessible via a network (e.g., remote server), wherein the computing device or remote server comprises one or more processors configured to execute instructions to perform operations comprising single cell DS analysis steps. For example, the processors may be configured to execute instructions for processing raw or unanalyzed sequencing reads to generate single cell DS or cDNA sequencing data. In additional embodiments, the computer program product may include a database comprising subject or sample records (e.g., information regarding a particular subject or sample or groups of samples) and empirically-derived information regarding known mutations, cell types. known biomarkers, phased mutations, epigenetic profiles, transcriptomes, etc. The computer program product is embodied in a non-transitory computer readable medium that, when executed on a computer. performs steps of methods disclosed herein.

The kits may further comprise include instructions and/or access codes/passwords and the like for accessing remote server(s) (including cloud-based servers) for uploading and downloading data (e.g., sequencing data, reports, other data) or software to be installed on a local device. All computational work may reside on the remote server and be accessed by a user/kit user via internet connection, etc.

### Selected Examples of Applications

As is described herein, provided methods may be used for any of a variety of purposes and/or in any of a variety of scenarios. Below are described examples of non-limiting applications and/or scenarios for the purposes of specific illustration only.

Of particular interest is the detection of one or more variants that includes a functionally disruptive mutation. In embodiments, provided methods are useful for detection of one or more variants in one or more cancer-associated genes, such as cancer driver genes. In embodiments, a cancer driver is a known cancer driver from Cancer Gene Census (CGC) or the COSMIC database (genes causally implicated in cancer). In embodiments, a cancer driver gene is or includes; ABL, ACC. BCR BLCA, BRCA. CESC. CHOL, COAD, DLBC. DNMT3A, EGFR, ESCA, GBM, HNSC, RICH, KIRC. KIRP. LAML, LGG. LIHC, LUAD. LUSC. MESO. OV, PAAD. PCPG. PI3K. PIK3CA, PRAD, PTEN READ, SARC, SKCM, STAD, TGCT. THCA, THYM, UCEC. UCS, and/or UVM. In embodiments, a cancer driver gene is or includes TP53. In embodiments, a cancer driver gene is or includes HRAS. NRAS, or KRAS.

In embodiments, if a variant is detected in one or more cancer drivers among a plurality of enriched tagged DNA molecules in the population of cells, then the method may further include a step of determining a variant frequency of the variant among the plurality of cells.

Accurate variant detection among cells may be used in a number of applications. The present technology relates generally to methods for detecting rare variants in nucleic acid sequences in a cell-specific or cell-identified manner. In embodiments, the methods provide for single cell analysis and interrogation, and associated reagents for use in such methods. Applications of cell specific variant detection (alone or in combination with a plurality of variants) include, but are not limited to, disease detection, disease-state assessment, disease risk assessment. cancer detection including early cancer detection, measurable residual disease (MRD) detection, identifying a cancer risk, identifying a treatment-resistant clone, identifying genotoxic or carcinogenic agents, monitoring cell therapy treatment, monitoring transplant rejection, etc.

Clonal hematopoiesis of indeterminate potential (CHIP) is a common aging-related phenomenon in which hematopoietic stem cells (HSCs) or other early blood cell progenitors contribute to the formation of a genetically distinct subpopulation of blood cells. Subpopulations formed by HSCs in the blood can be characterized by a shared unique mutation profile that is carried forward in daughter cells of the clones. In embodiments, single-cell DS can be used to interrogate blood cell populations to better understand, or map, clones derived from HSCs. In other embodiments, age-related disease states can be identified with single cell DS analysis, e.g., as disclosed herein.

In embodiments, provided methods and compositions allow for the detection of mutations in a population of cells within a biological sample, wherein the mutations are associated with a specific cell type or a particular cell. Accordingly, aspects of the present technology can interrogate multiple cells in a high throughput manner and provide both RNA (e.g., transcriptome) and DNA derived sequencing information with a high level of sensitivity and specificity. In embodiments, the number of cells that can be interrogated by the methods and systems disclosed herein can be in the 10s, the 100s, the 1000s, the 10.000s, the 100.000s, or in the 1.000.000s.

In embodiments, a disease state or a disease-associated mutation is identified as actionable (e.g., via treatment) because the particular identified mutation or combination of mutations are associated with a particular cell type.

It is contemplated that nucleic acid material may come from a variety of sources. For example, in embodiments, nucleic acid material (e.g., comprising one or more double-stranded nucleic acid molecules) is provided from a sample from a human subject, an animal, a plant, a fungi, a virus, a bacterium, a protozoan or any other life form. In other embodiments, the sample comprises nucleic acid material that has been at least partially artificially synthesized. In embodiments, a sample is or comprises a body tissue, a biopsy, a skin sample, blood, serum, plasma, sweat, saliva, cerebrospinal fluid, mucus, uterine lavage fluid, a vaginal swab, a pap smear, a nasal swab, an oral swab, a tissue scraping, hair, a finger print, urine, stool, vitreous humor, peritoneal wash, sputum, bronchial lavage, oral lavage, pleural lavage, gastric lavage. gastric juice, bile, pancreatic duct lavage, bile duct lavage, common bile duct lavage, gall bladder fluid, synovial fluid, an infected wound, a non-infected wound, an archaeological sample, a forensic sample, a water sample, a tissue sample, a food sample, a bioreactor sample, a plant sample, a bacterial sample, a protozoan sample, a fungal sample, an animal sample, a viral sample, a multi-organism sample, a fingernail scraping, semen, prostatic fluid. vaginal fluid, a vaginal swab, a fallopian tube lavage, a cell free nucleic acid, a nucleic acid within a cell, a metagenomics sample, a lavage or a swab of an implanted foreign body, a nasal lavage, intestinal fluid, epithelial brushing, epithelial lavage, tissue biopsy, an autopsy sample, a necropsy sample, an organ sample, a human identification sample, a non-human identification sample, an artificially produced nucleic acid sample, a synthetic gene sample, a banked or stored nucleic acid sample, tumor tissue, a fetal sample, an organ transplant sample, a microbial culture sample, a nuclear DNA sample, a mitochondrial DNA sample, a chloroplast DNA sample, an apicoplast DNA sample, an organelle sample, and any combination thereof. In embodiments, the nucleic acid material is derived from more than one source.

In various applications, the present technology can be used to screen or monitor a subject for disease-associated mutations within select cell types for (a) an early detection of cancer, (b) detection of minimal residual disease, (c) assessing a cancer risk, (d) stratifying a subject (e.g., patient) for determining a recommended treatment course, etc. In embodiments, a cancer detection screening can be a stand-alone screening test. For example, a healthy subject can exhibit no symptoms of a disease state and provide a biological sample for single cell DS screening for a disease-associated mutation within a target cell type.

In other embodiments, a subject may have had an early cancer detection liquid biopsy test that provided an indication of a presence of a cancer-associated mutation in a cell-free DNA (cfDNA) sample, but the test was unable to provide the specific cell type to appreciate if the mutation was indeed associated with an actual disease state in the subject. In accordance with embodiments of the present technology, a single cell DS assay can be used on one or more biological samples to determine a tissue/cell source of the cancer-associated mutation.

In a particular embodiment, the biological sample is a uterine lavage fluid. In embodiments, the uterine lavage can provide about 10,000 to about 1,500,000 cells to interrogate. In embodiments, about 100 ng to about 10 μg of genomic DNA is interrogated from cells isolated from the uterine lavage fluid. In other embodiments, the sample can comprise fewer or more cell numbers. In accordance with embodiments disclosed herein, a single cell DS assay can be used to determine if there is a disease-associated mutation present in one or more cells from the uterine lavage. In embodiments, the disease-associated mutation can be in one or more of TP53, BRCA1/2, PIK3CA, and KRAS. Transcriptome analysis can be used to determine a cell type associated with an identified disease-associated mutation. In embodiments, the disease-associated mutation is in a cell associated with a gynecological cancer (e.g., ovarian cancer, cancer of the fallopian tube cells, endometrial cancer, uterine cancer, etc.). Optionally, the assay can be used to determine the presence or absence of a disease state within the subject based on the cell-type of the cell and/or cellular organelle from which the disease-associated mutation was derived. If the disease-state is identified, it is contemplated that appropriate therapeutic treatment could be provided.

In another embodiment, the biological sample is a urine sample, and a single cell DS assay can be used to determine if there is a disease-associated mutation present in one or more cells collected from the urine sample. Transcriptome analysis can be used to determine a cell type associated with an identified disease-associated mutation. In embodiments, the disease-associated mutation is in a cell associated with a bladder cancer. In other embodiments, the disease-associated mutation is in a cell associated with a prostate cancer. Optionally, the assay can be used to determine the presence or absence of a disease state within the subject based on the cell-type of the cell and/or cellular organelle from which the disease-associated mutation was derived. If the disease-state is identified, it is contemplated that appropriate therapeutic treatment could be provided.

In yet another embodiment, the biological sample is a blood sample, and a single cell DS assay can be used to determine if there is a disease-associated mutation present in one or more cells collected from the blood sample. Transcriptome analysis can be used to determine a cell type associated with an identified disease-associated mutation. In embodiments, the disease-associated mutation is in a cell associated with a leukemia, a lymphoma or a myeloma. Optionally, the assay can be used to determine the presence or absence of a disease state within the subject based on the cell-type of the cell and/or cellular organelle from which the disease-associated mutation was derived. If the disease-state is identified, it is contemplated that appropriate therapeutic treatment could be provided.

In a further embodiment, the biological sample is a gastric lavage sample, and wherein a single cell DS assay is used to determine a cell type associated with an identified disease-associated mutation. In embodiments, the disease-associated mutation is in a cell associated with a gastric cancer. In another embodiment, a single cell DS assay can be used to associate mutations identified in oral tissue scrapings with an oral cancer. In a further embodiment, a stool sample can be subjected to single cell DS interrogations and a disease-associated mutation can reveal a colon cancer.

In other embodiments, a medical disorder is treated by administration of a genome-edited immune effector cell (e.g., a T cell) that elicits a specific immune response. In embodiments, cells for use in a therapeutic application may be propagated for days, weeks, or months ex vivo as a bulk population within about 1, 2, 3, 4. 5 days or more following a genome editing event. In embodiments, genome edited cells may be obtained from a subject after administration and analyzed. For example, in the case of an engineered immune cell, such edited cells may be obtained from the blood of a treated subject and characterized using the single cell DS methods of the present disclosure. In embodiments, abundance of one or more variants in a cell population is monitored over time. In embodiments, clonal expansion of one or more variants in a cell population is monitored over time.

In yet another embodiment, a single cell DS assay can be used to identify a fetal cell in population of maternal cells. In a further embodiment, a single cell DS assay can be used to identify a transplant rejection in a patient (e.g., at earlier time points than currently available assays).

In other embodiments, a variant frequency or mutation frequency of particular mutations can be assessed from a biological sample. In embodiments, the genotoxicity or carcinogenicity of an agent (e.g., on a test animal, cell culture or other subject) can be assessed efficiently and with tissue specificity (e.g., liver, bone, brain, lung, blood, etc.) using single cell DS as described in the embodiments presented herein.

### Embodiments of Systems and Computing Environments for Characterization of Cell Specific Nucleic Acids

### Suitable Computing Environments

The following discussion provides a general description of a suitable computing environment in which aspects of the disclosure can be implemented. Although not required, aspects and embodiments of the disclosure will be described in the general context of computer-executable instructions, such as routines executed by a general-purpose computer, e.g., a server or personal computer. Those skilled in the relevant art will appreciate that the disclosure can be practiced with other computer system configurations, including Internet appliances, handheld devices, wearable computers, cellular or mobile phones, multi-processor systems, microprocessor-based or programmable consumer electronics, set-top boxes, network PCs, mini computers, mainframe computers and the like. The disclosure can be embodied in a special purpose computer or data processor that is specifically programmed, configured or constructed to perform one or more of the computer-executable instructions explained in detail below. Indeed, the term “computer”, as used generally herein, refers to any of the above devices, as well as any data processor.

The disclosure can also be practiced in distributed computing environments, where tasks or modules are performed by remote processing devices, which are linked through a communications network, such as a Local Area Network (“LAN”), Wide Area Network (“WAN”) or the Internet. In a distributed computing environment, program modules or sub-routines may be located in both local and remote memory storage devices. Aspects of the disclosure described below may be stored or distributed on computer-readable media, including magnetic and optically readable and removable computer discs, stored as firmware in chips (e.g., EEPROM chips), as well as distributed electronically over the Internet or over other networks (including wireless networks). Those skilled in the relevant art will recognize that portions of the disclosure may reside on a server computer, while corresponding portions reside on a client computer. Data structures and transmission of data particular to aspects of the disclosure are also encompassed within the scope of the disclosure.

Embodiments of computers, such as a personal computer or workstation, can comprise one or more processors coupled to one or more user input devices and data storage devices. A computer can also be coupled to at least one output device such as a display device and one or more optional additional output devices (e.g., printer, plotter, speakers, tactile or olfactory output devices, etc.). The computer may be coupled to external computers, such as via an optional network connection, a wireless transceiver, or both.

Various input devices may include a key board and/or a pointing device such as a mouse. Other input devices are possible such as a microphone. joystick, pen, touch screen, scanner, digital camera, video camera, and the like. Further input devices can include sequencing machine(s) (e.g., massively parallel sequencer), fluoroscopes, and other laboratory equipment, etc. Suitable data storage devices may include any type of computer-readable media that can store data accessible by the computer, such as magnetic hard and floppy disk drives, optical disk drives, magnetic cassettes, tape drives, flash memory cards, digital video disks (DVDs). Bernoulli cartridges, RAMs, ROMs, smart cards, etc. Indeed, any medium for storing or transmitting computer-readable instructions and data may be employed, including a connection port to or node on a network such as a local area network (LAN), wide area network (WAN) or the Internet.

Aspects of the disclosure may be practiced in a variety of other computing environments. For example, a distributed computing environment with a network interface can include one or more user computers in a system where they may include a browser program module that permits the computer to access and exchange data with the Internet, including web sites within the World Wide Web portion of the Internet. User computers may include other program modules such as an operating system, one or more application programs (e.g., word processing or spread sheet applications), and the like. The computers may be general-purpose devices that can be programmed to run various types of applications, or they may be single-purpose devices optimized or limited to a particular function or class of functions. More importantly, while shown with network browsers, any application program for providing a graphical user interface to users may be employed, as described in detail below; the use of a web browser and web interface are only used as a familiar example here.

At least one server computer, coupled to the Internet or World Wide Web (“Web”), can perform much or all of the functions for receiving, routing and storing of electronic messages, such as web pages, data streams, audio signals, and electronic images that are described herein. While the Internet is shown, a private network, such as an intranet may indeed be preferred in some applications. The network may have a client-server architecture, in which a computer is dedicated to serving other client computers, or it may have other architectures such as a peer-to-peer, in which one or more computers serve simultaneously as servers and clients. A database or databases, coupled to the server computer(s), can store much of the web pages and content exchanged between the user computers. The server computer(s), including the database(s), may employ security measures to inhibit malicious attacks on the system, and to preserve integrity of the messages and data stored therein (e.g., firewall systems, secure socket layers (SSL), password protection schemes, encryption, and the like).

A suitable server computer may include a server engine, a web page management component, a content management component and a database management component, among other features. The server engine performs basic processing and operating system level tasks. The web page management component handles creation and display or routing of web pages. Users may access the server computer by means of a URL associated therewith. The content management component handles most of the functions in the embodiments described herein. The database management component includes storage and retrieval tasks with respect to the database. queries to the database, read and write functions to the database and storage of data such as video. graphics and audio signals.

Many of the functional units described herein have been labeled as modules, in order to more particularly emphasize their implementation independence. For example, modules may be implemented in software for execution by various types of processors. An identified module of executable code may, for instance, comprise one or more physical or logical blocks of computer instructions which may, for instance, be organized as an object, procedure, or function. The identified blocks of computer instructions need not be physically located together but may comprise disparate instructions stored in different locations which, when joined logically together. comprise the module and achieve the stated purpose for the module.

A module may also be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

A module of executable code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network.

### System for Characterizing Cell Specific Sequencing Data

The present technology further comprises a system (e.g, a networked computer system, a high throughput automated system, etc.) for processing a biological sample comprising a nucleic acid mixture, and transmitting the sequencing data via a wired or wireless network to a server to determine the sample's error-corrected sequence reads (e.g., duplex sequence reads, duplex consensus sequence, etc.), cell-specific transcriptome sequence reads (cDNA sequence reads), error-corrected sequence reads at target regions (e.g., cancer driver loci), groups of sequence reads associated with a specific cell (e.g., error-corrected sequence reads, cDNA sequence reads), reference sequences, variant identification, variant frequency, quantification of cell specific/attributable genotypes, and the like.

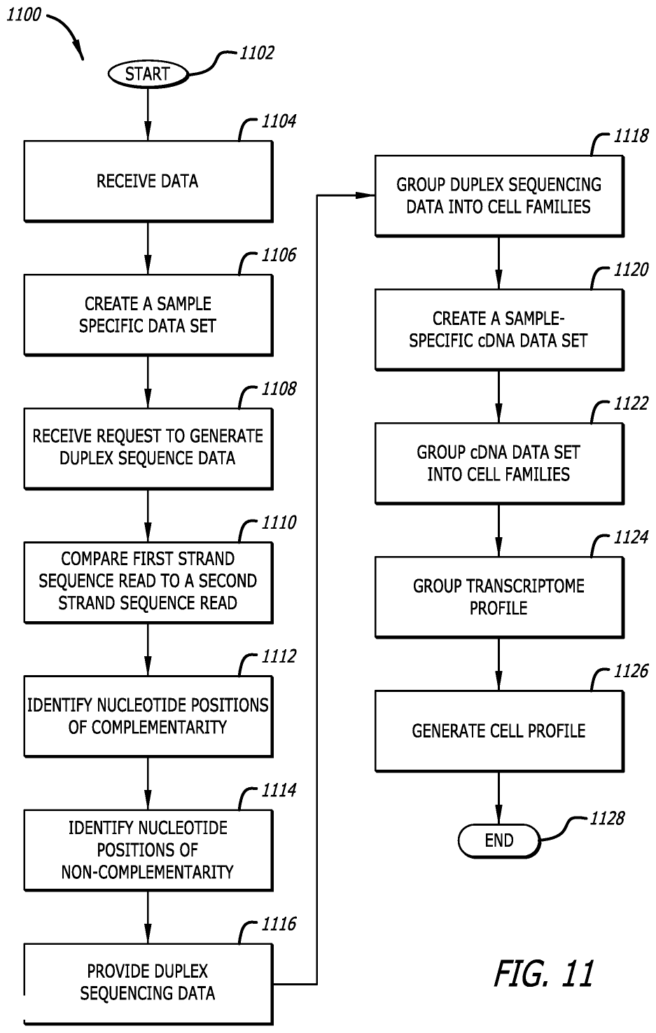

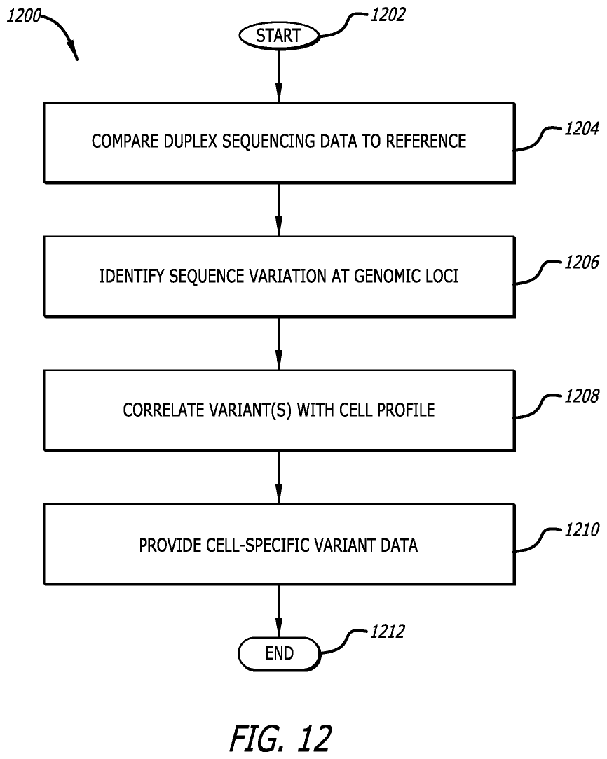

In embodiments, a computerized system for characterization of nucleic acids of a cell population comprises; (1) a server (e.g., a remote server, or locally stored server); (2) a plurality of user electronic computing devices able to generate and/or transmit sequencing data; (3) optionally, a database with reference sequences (e.g., expected genomic sequences, anticipated genomic sequences, a subject's prior sequence reads, genomic sequence derived from a healthy subject, etc.) and associated information (optional); and (4) a wired or wireless network for transmitting electronic communications between the electronic computing devices, database, and the server. The server further comprises; (a) a database storing sequence record results, and records of variant profiles (e.g, mutation profiles, variant frequency results, etc.); (b) one or more processors communicatively coupled to a memory; and one or more non-transitory computer-readable storage devices or medium comprising instructions for processor(s), wherein said processors are configured to execute said instructions to perform operations comprising one or more of the steps described in FIGS. 11-12.

In embodiments, the present technology further comprises a non-transitory computer-readable storage media comprising instructions that, when executed by one or more processors. performs methods for determining the presence of a sequence variant in population of cells, determining a presence of a variant(s) within a particular cell or cell type, determining a combination of variants present within a particular cell or cell type, determining a cell type for one or more cells in a population of cells, determining the presence of one or more variants in a target genomic region (e.g., cancer driver or non-cancer driver loci), determining a variant frequency at a particular genomic locus among a cell population, determining a mutation frequency at a particular genomic locus among a cell population, determine a variant frequency for a combination of variants present in a cell population, and the like.

Additional aspects of the present technology are directed to computerized methods for determining the presence of a sequence variant in population of cells, determining a presence of a variant(s) within a particular cell or cell type, determining a combination of variants present within a particular cell or cell type, determining a cell type for one or more cells in a population of cells, determining the presence of one or more variants in a target genomic region (e.g., cancer driver or non-cancer driver loci), determining a variant frequency at a particular genomic locus among a cell population, determining a mutation frequency at a particular genomic locus among a cell population, determine a variant frequency for a combination of variants present in a cell population, and the like.

FIGS. 11-12 are flow diagrams illustrating various routines for identifying and/or quantifying variants (e.g., variants, combinations of variants, mutations, combination of mutations, disease associated mutations, etc.) within particular cells in a cell population. In accordance with aspects of the present technology, methods described with respect to FIGS. 11-12 can provide sample data including, for example, genetic profiles of single cells within a cell population (e.g., a cell population from a biological sample), including genome variants present in a cell, transcriptome data of a cell. variants in cancer drivers that would suggest a disease state in one or more cells and/or the subject from which a biological sample was derived, and information derived from comparison of sample data to data sets of reference sequences.

A suitable computing system invokes a number of routines. While some of the routines are described herein, one skilled in the art is capable of identifying other routines the system could perform. Moreover, the routines described herein can be altered in various ways. As examples, the order of illustrated logic may be rearranged, substeps may be performed in parallel, illustrated logic may be omitted, other logic may be included, etc.

FIG. 11 is a flow diagram illustrating routine 1100 for providing Duplex Sequencing Data for double-stranded nucleic acid molecules in a cell (e.g., a cell from a biological mixture). The routine 1100 can be invoked by a computing device, such as a client computer or a server computer coupled to a computer network. As an example, the computing device may invoke the routine 1100 after an operator engages a user interface in communication with the computing device.

The routine 1100 begins at block 1102 and a sequence module receives raw sequence data from a user computing device (block 1104) and creates a sample-specific data set comprising a plurality of raw sequence reads derived from a plurality of nucleic acid molecules in the sample (block 1106). In embodiments, the server can store the sample-specific data set in a database for later processing. Next, a DS module receives a request for generating Duplex Consensus Sequencing data from the raw sequence data in the sample-specific data set (block 1108). The DS module groups sequence reads from families representing an original double-stranded nucleic acid molecule (e.g., based on UMI sequences) and compares representative sequences from individual strands to each other (block 1110). In embodiments, the representative sequences can be one or more than one sequence read from each original nucleic acid molecule. In another embodiment, the representative sequences can be single-strand consensus sequences (SSCSs) generated from alignment and error-correction within representative strands. In such embodiments, a SSCS from a first strand can be compared to a SSCS from a second strand.

At block 1112, the DS module identifies nucleotide positions of complementarity between the compared representative strands. For example, the DS module identifies nucleotide positions along the compared (e.g., aligned) sequence reads where the nucleotide base calls are in agreement. Additionally, the DS module identifies positions of non-complementarity between the compared representative strands (block 1114). Accordingly, the DS module can identify nucleotide positions along the compared (e.g., aligned) sequence reads where the nucleotide base calls are in disagreement.

Next, the DS module can provide Duplex Sequencing Data for double-stranded nucleic acid molecules in a sample (block 1116). Such data can be in the form of duplex consensus sequences for each of the processed sequence reads. Duplex consensus sequences can include, in embodiments, only nucleotide positions where the representative sequences from each strand of an original nucleic acid molecule are in agreement. Accordingly, in embodiments, positions of disagreement can be eliminated or otherwise discounted such that the duplex consensus sequence is a high accuracy sequence read that has been error-corrected. In another embodiment, Duplex Sequencing Data can include reporting information on nucleotide positions of disagreement in order that such positions can be further analyzed (e.g., in instances where DNA damage can be assessed). At block 1118, the DS module groups duplex consensus sequences into cell families representing an original cell (e.g., based on a cell index).

At block 1120, the sequencing module receives cDNA raw sequencing reads from a user computing device and creates a sample-specific cDNA data set comprising a plurality of raw sequence reads derived from a plurality of cDNA in the sample. At block 1122, a transcriptome module groups sequence reads from families representing an original double-stranded cDNA molecule (e.g., based on UMI sequences) and receives a request for grouping cDNA sequence reads into the cell families representing an original cell (e.g., based on a cell index). At block 1126, the transcriptome module (block 1124). Next, the transcriptome module determines a cell profile (e.g., cell type, cell state, etc.) from the transcriptome profile (block 1126). The routine may then continue at block 1128, where it ends.

FIG. 12 is a flow diagram illustrating a routine 1200 for detecting and identifying variant(s) in a cell in a cell population. The routine 1200 begins at block 1202 and the variant module compares the Duplex Sequencing Data from FIG. 11 (e.g., following block 1116) to reference sequence information (block 1204) and identifies sequence correspondence and/or variation (e.g., where the subject sequence corresponds with a reference sequence, where the subject sequence varies from the reference sequence, etc.) at one or more genomic loci (block 1206).

Next, the variant module correlates variant(s) identified in a cell family with the cell profile of the cell family (block 1208) As such, a variant analysis can be provided with information regarding the type of variant(s) (e.g., disease-associated variants, non-disease associated variants), and type of cell harboring the variant(s). Next, the variant module can provide cell-specific variant data (block 1210) that can be stored in the sample-specific data set in the database. The routine may then continue at block 1212. where it ends.

Other steps and routines are also contemplated by the present technology. For example, the cell profile may include other cellular information such as epigenetic data, spatial data and the like. In another example, the system can be configured to identify a disease state of a subject (source of biological sample) based on the cell-specific variant data. Other steps may include determining if a subject should be prophylactically or therapeutically treated based on an identified disease state. Further steps may include determining if a subject should be therapeutically treated for cancer based on the cell-specific variant data derived from a particular subject's biological sample. If so, then disease treatments may be initiated.

### EXAMPLES

By example and without limitation, embodiments are described according to the following examples;

### Example 1. Combinatorial Cell Indexing Mediated Single Cell DS

The present Example describes the use of combinatorial cellular barcoding with Duplex Sequence (scDuplex-Seq) to detect low frequency mutations in individual cells. In the present example, isolated nuclei were used instead of whole cells. It is known in the art that isolated nuclei can be used in lieu of whole cells in single-cell applications concerning genomic DNA. In the present Example, a mixture of cells culture lines from mice (T3T cell) and humans (HEK293) cells were used.

### Nuclei Fixation and Nucleosome Depletion

In the present Example, the following nucleosome depletion steps are adapted from Vitak et al. (Sequencing thousands of single-cell genomes with combinatorial indexing. Nat Methods. 2017; 14 (3); 302-308) which is incorporated herein by reference in its entirety. All centrifuge steps are performed in a swinging bucket centrifuge to reduce cell shearing. An initial cell pellet of either cultured cell lines or primary cells were resuspended in 10 ml of cell media (DMEM. 10% FBS). 406 μl of 37% formalin were then added to the cell suspension and incubated for 10 minutes with gentle shaking. To neutralize reaction, 1 mL of ImM Tris (8.0 pH) was added and cell solution was then centrifuged at 550×g for 8 minutes at 4° C. Cells were then resuspended in 10 mL of cold 1×PBS and centrifuged again for 8 minutes at 550× g. Cells were then resuspended in 5 mL of ice cold nuclear isolation buffer (10 mM Tris-HCl PH 7.4, 10 mM NaCl, 3 mM MgCl2. 0.1% Igepal. 1× protease inhibitors (Roche. Cat. 1873580001). Cells were incubated for 20 minutes on ice with gentle shaking, then pelleted again at 500× g for 5 minutes. Cells were washed with 900 μl 1× NEBuffer 2.1 and pelleted again at 500× g for 5 minutes. Cells were resuspended in 800 uL 1× NEBuffer 2.1 with 24 μl of 10% SDS and transferred to a 1.7 mL microcentrifuge tube. Cells were then incubated at 42° C., for 30 minutes with vigorous shaking. ul of 10% Triton-X were then added and cells were incubated for another 30 minutes at 42° C. with shaking. Cells were then pelleted at 500× g for 3 minutes at 4° C. Cells were then resuspended in 300 μL of cold 0.5× PBS. Cells were then strained through a 40 μm strainer into a new low-bind microcentrifuge tube, counted and diluted down to 1 million cells per mL.

### Genome Fragmentation and End-Prep.

In the present Example, fixed and nucleosome depleted nuclei were spun down at 550× g for 8 minutes and resuspended in 35 μl 0.5× PBS and transferred to a PCR strip tube. 5 μl of H20. 5 μL of rCutSmart™ Buffer (New England Biolabs. Ipswitch MA) and Sul of the restriction enzyme HpyCH4V enzyme (New England Biolabs, Ipswitch MA) were then added to digest nucleosome depleted gDNA. Nuclei were then digested at 37° C., for 1 hour followed by a heat inactivation at 65° C., for 20 minutes. Nuclei were then pelleted at 1500× g for 5 minutes and washed with Tris/BSA (995 μL Qiagen EB+5 μL of 20 mg/mL BSA) and pelleted. Nuclei were then resuspended in pre-mixed Ultra II Kit End Repair buffer (New England Biolabs. Ipswitch MA); 49.7 μL water+7 μL buffer+0.3 μL BSA. 3 μL of Ultra II End Repair/A-tail enzyme mix (New England Biolabs, Ipswitch MA) was then added to this mix, pipetted to mix and incubated at 20° C., for 30 minutes then heat inactivated at 65° C., for 30 minutes.

### Ligation of Cellular Indexes

In the present Example, to the nuclei suspension. 30 μl of Ultra II ligation mix and lul of Ultra Il enhancer were then added (New England Biolabs. Ipswitch MA). 15 μl of this 91 μl suspension was then distributed to each of 6 wells of a 96-well plate containing 8 μL of 12.5 uM of round 1 cellular indexes each. Nuclei/Adapter mix was then incubated for 30 minutes at 20° C. Nuclei were then pooled and spun down 1500g for 5 minutes. Nuclei were resuspended in 2 mL of 1× NEB 3.1 buffer (New England Biolabs, Ipswitch MA). This nuclei mixture was then added to a 2.04 mL mixture of 1× T4 Ligase Buffer. 0.2 mg/mL BSA, and 8U/uL of T4 DNA Ligase (New England Biolabs, Ipswitch MA). This 4 mL mixture was then added to a basin and 40 uL added to each well of a plate containing a 10 μl mixture of 12 uM round 2 combinatorial indexes and 11 uM round 2 linker. This plate was then sealed with an adhesive plate seal and incubated in a thermal cycler for 30 minutes at 37° C. 10 μl of Round 2 blocking solution (26.4 uM blocking oligo in 1× Ligase buffer, total volume 1200 μL) was then added to each well. Plates were then incubated for an additional 20 minutes at 37° C. Nuclei were then pooled into a new basin and passed through a 40 μm strainer into a new basin. An additional 20 μl of T4 DNA ligase (2,000U/ul) (New England Biolabs, Ipswitch MA) was then added to the basin and mixed by pipetting approximately 20 times. 50 μl of this mixture was then added to each well of round 3 combinatorial barcode plate containing 10 μl mixture of 14 uM round 3 combinatorial indexes and 13 uM round 3 linker. Plates were then incubated for 30 minutes at 37° C. 20 μl of round 3 blocking solution (11.5 uM blocking oligo with 125 mM EDTA in water) was then added to each well. All nuclei were then pooled into a new basin and passed through a 40 μm strainer into a 15 mL falcon tube.

### Nuclei Sublibrary Preparation and Lysis

In the present Example, 70 μl of 10% Triton-X were then added to nuclei mix to a final concentration of approximately 0.1%. Nuclei were then centrifuged at 1000×g for 5 minutes in 15 mL tube. Supernatant was removed leaving about 30 μl behind to avoid disturbing nuclei pellet. Nuclei were then washed twice with 4 mL of wash buffer (1×PBS, 0.1% Triton-X). Supernatant was then aspirated, and nuclei were resuspended in 1× PBS up to a total volume of 100 μL. Nuclei were then counted, and the number of nuclei desired for each sub-library were then aliquoted into fresh 1.7 ml microcentrifuge tubes and brought up to 50 μL with 1× PBS. To each sublibrary to be processed add 50 μL of 2× Lysis Buffer (20 mM Tris pH 8.0, 400 mM NaCl, 100 mM EDTA ph 8.0. 4.4% SDS). To each tube 10 μL of Proteinase K (20 mg/mL) was then added. Sublibraries were then incubated at 55° C., for two hours while shaking at 200 rpm.

### Purification of DNA

In the present Example, for each lysate to be processed, 44 μl of MyOne Cl Dynabeads (Thermo-Fisher) were added to a 1.ml tube and 800 ul of 1×B&W-T buffer (5 mM Tris-HCl, pH 7.5. 1M NaCl, 0.5 mM EDTA, 0.05% Tween-20) was then added. Beads were then placed against a magnetic rack until clear. The supernatant was discarded and beads were washed again with ul of 1×B&W-T three times. Beads were then resuspended in 100 μl per sample of 2×B&W buffer (10 mM Tris-HCl pH 7.5, 2M NaCl. ImM EDTA). Lysates were then warmed to 37° C. 5 μL of proteinase inhibitor cocktail AEBSF were then added to lysates which were then left at room temperature for 10 minutes. 100 μl of resuspended beads were then added to each lysate. DNA was bound to beads by agitating at room temperature for 60 minutes. Samples were then placed against a magnet and supernatant was removed then beads were resuspended with 250 μl of 1×B&W-T buffer and agitated for a further 5 minutes at room temperature. This step was then repeated to wash the beads again with 1×B&W-T. Beads were resuspended a final time in Tris-T buffer (9.9 mM Tris-HCl pH 8.0, 0.01% Tween-20) and agitated for 5 minutes at room temperature.

Extension with BST Polymerase and Second Genome Fragmentation

In the present Example, the test sample was then resuspended in 50 μl Thermopol Bst mix (1× Thermopol Buffer, 100 uM dNTPs, 1U BST polymerase) (New England Biolabs, Ipswitch MA) and incubated at 60° C., for 30 minutes. Samples were then placed on a magnet and supernatant was removed. Samples were then resuspended in 35 μl of fragmentation mixture (26 μl H20. 7 μL NEB Ultra II FS reaction buffer. 2 μl NEB Ultra II FS enzyme mix) (New England Biolabs. Ipswitch MA). Samples were then incubated for 4 minutes at 37° C., and heat inactivated at 65° C. for 30 minutes then placed on ice. Truseq adapter oligos (IlluminaR, San Diego CA) were then annealed by adding 9.5 μl of each 100 uM adapter to a PCR strip tube, with 1 ul of 1M NaCl and annealed in a thermocycler by dropping the temperature from 95° C., to 20° C., at −0.1° C./s. This mixture was then diluted with water to 12 uM. Bead mixture was then transferred to a 1.7 mL microcentrifuge tube and 30 ul of Ultra II ligation mix and lul of enhancer were added to mixture (New England Biolabs, Ipswitch MA). 12 μl of diluted adapter was then added to this mix and ligated at room temperature for 30 minutes.

### DNA Amplification

Samples were placed against a magnet and supernatant was removed. Samples were then washed with 250 μl of nuclease-free water without resuspending beads. Beads were then resuspended in 220 μl of PCR mix (1× Kapa Hifi master mix, 0.2 uM p5 universal primer, 0.2 uM p7 indexing primer) (Roche). This mixture was then split across 4 PCR strip tubes and run on a thermocycler with the following parameters; 95° C., for 3 minutes. 5 cycles of 98° C., for 20 seconds. 65° C., for 45 seconds, and 72° C., for 1 minute and 30 seconds. Reactions were then combined again into a single microcentrifuge tube. Samples were then place against a magnet and the supernatant was retained. 200 μl of this supernatant was split across four optical grade pcr tubes (50 μl each) for qPCR. 2.5 μl of 20× Evagreen was added to this mixture. These samples were then run for 5-additional qPCR cycles and removed during the exponential phase according to qPCR trace.

### Sequencing

In the present Example, a specific genomic region corresponding to TP53 was enriched with targeted hybridization capture using biotinylated DNA oligonucleotide probes specific to those regions using a commercially available target enrichment kit optimized for these oligonucleotide probes (TwinStrand Biosciences. Seattle WA). After targeted enrichment, the captured DNA was PCR amplified using a commercially available high-fidelity DNA polymerase (TwinStrand Biosciences, Seattle WA) and sequenced on an IlluminaR NextSeq550 sequencer and devoting ˜150×106 paired end reads with a read configuration of 190-cycles for Read 1 and 110-cycles for Read 2 and a single 8-cycle indexing read. Read 1 encompassed the genomic DNA and Read 2 encompassed the entirety of the combinatorial barcoding sequences.

### Data Processing

A custom bioinformatic pipeline was created to automate the analysis from raw FASTQ files to text files. This pipeline is similar to methods used for standard Duplex Sequencing and SPLIT-Seq analysis that have been previously published (Sanchez-Contreras et al. Nucleic Acids Res. 49 (19); 11103-11118. 2021 & Rosenburg et al. Science. 360 (6385); 176-182) but with the following several modifications; 1) The combinatorial barcodes (three of them), UMI, and SDE (derived from the ‘wobble’ base design were parsed from one of the reads (Read 2) and associated with corresponding read sequence in Read I in a special user defined field in an unaligned BAM file; 2) Consensus making was performed differently. Specifically, consensus make was performed by first grouping all reads sharing a combinatorial barcode and then performing consensus making on reads that share a UMI sequence and SDE sequence. In the present Example complementary sequences are all found in the read end (i.e., . Read 1 end). Strand identification is achieved, not by the read end in which complementary sequences are located, but by the sequence of the SDE tag in the user defined field in an unaligned bam file (e.g., . Top strand orientation; TTT vs Bottom strand orientation; GGG). It is considered more likely than not that this same approach could be performed on an aligned BAM file and with different SDE sequences.

Consensus making was executed by a custom Python script, which grouped all the reads that share the same combinatorial barcode located in the user-defined field in the unaligned BAM file. After combinatorial barcode grouping, reads sharing the same UMI sequence and same SDE sequence were compared the base called at each position and produced a single-stranded consensus sequence (SSCS) read. The SSCS reads for each pair of tags with opposite sequence (TTT vs GGG in the present Example) were then compared position by position to create a double-stranded consensus sequence (DCS) read). A single FASTQ files was made containing the resulting SSCS reads and DCS reads. The resulting single FASTQ file for the DCS reads was then aligned against the human reference genome (hg38) using the publicly available program bwa-mem v0.7.419 with default parameters. Next, sequences on the 3′ end of the reads corresponding to the ligated adapter sequence was removed by the publicly available program clipadapt. Lastly, the resulting BAM file was filtered to remove reads not aligning to the genomic positions of interest using a BED file. The BED file can be easily created using the coordinates of the target hybridization probes.

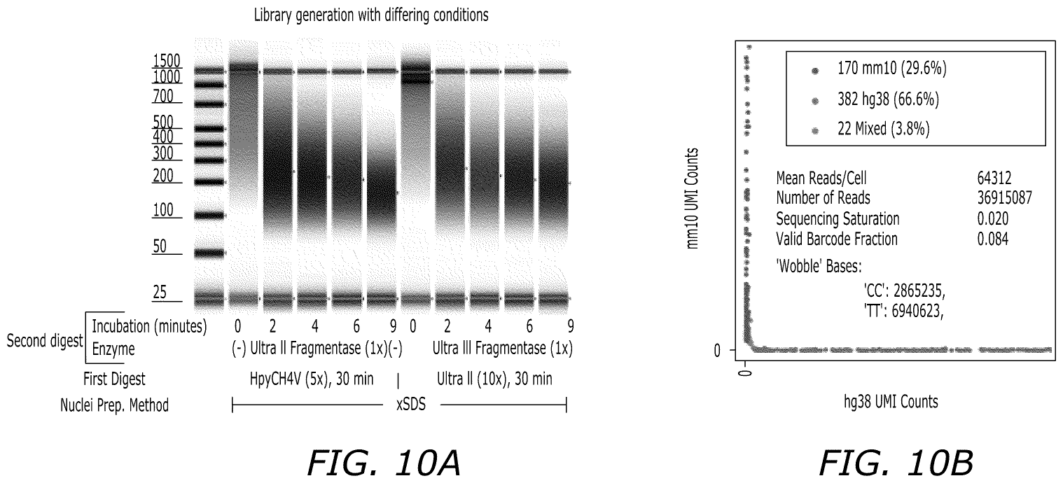

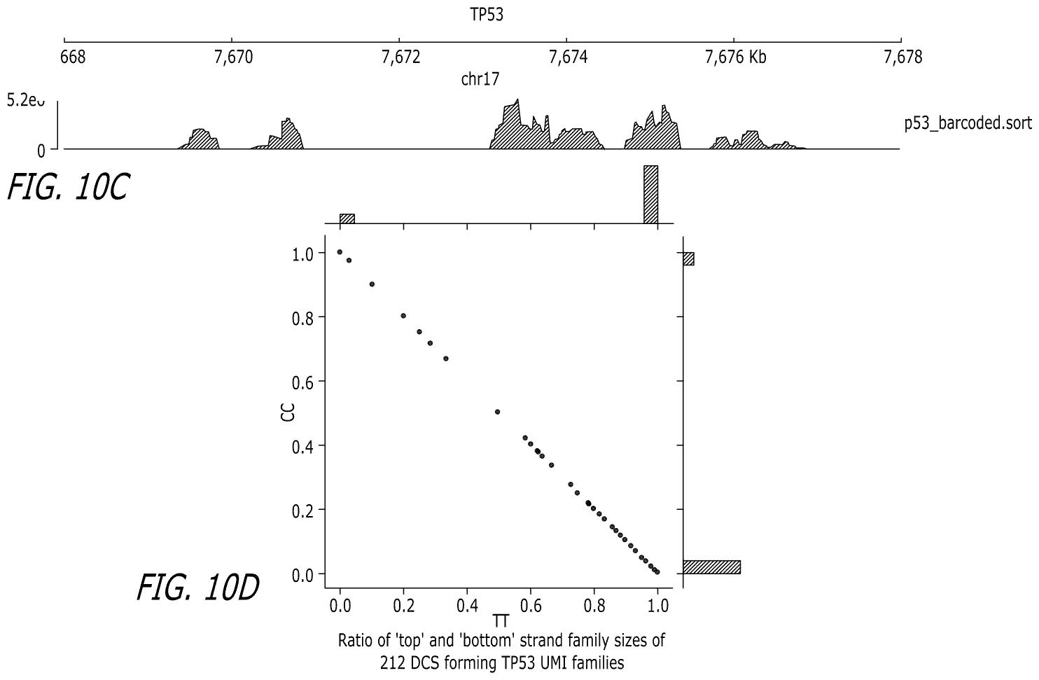

The present Example demonstrates the ability to enzymatically fragment the gDNA, in intact nuclei that is able to undergo end-repair, dA-tailing, and adapter ligation (FIG. 10A). The present Example further demonstrates that the resulting library can be enriched for the target region of TP53 and then sequenced (FIG. 10C). In addition, the present Example demonstrates that the gDNA from each nuclei can be attached to a unique combination of combinatorial barcodes corresponding to individual nuclei. Specifically, these combinatorial barcodes can be parsed from the reads and the reads grouped according to their combinatorial barcode specific for each nuclei such that the vast majority of reads can be discerned from the specific species (Mouse vs. Human) from which they were derived (FIG. 10B). Lastly, reads that share a UMI that map to the genomic locus of interest (i.e. TP53) can form both an SSCS and DCS read due to the presence of the SDE (FIG. 10D). A DCS can only be formed from an SSCS read can only be formed when two SSCS reads that share a UMI sequence and opposite SDE sequences.

### Examples of scDuplex Reagents

PBS (Thermo Fisher Scientific, 10010049).
Enzymatic RNase Inhibitor (Qiagen Enzymatics, Y9240L).
SUPERase In RNase Inhibitor (Thermo Fisher Scientific, AM2696).
NxGen RNase Inhibitor (Lucigen, 30281-2).
16% Formaldehyde (w/v) (Thermo Fisher Scientific, 28906).
Glycine (Sigma-Aldrich, 50046)
1M Tris HCl pH 7.5 (Thermo Fisher Scientific, 15567027).
1M Tris HCl pH 8.0 (Thermo Fisher Scientific, 15568025).
5M NaCl (Thermo Fisher Scientific. AM9760G).
1M MgCl2 (Sigma-Aldrich, 63069).
10% NP40 (Thermo Fisher Scientific, 28324).
Buffer EB (Qiagen. 19086).
DNA Clean & Concentrator-5 (Zymo, D4014)
PEG 6000 (Sigma-Aldrich,528877-100GM).
Maxima H Minus Reverse Transcriptase with buffer (Thermo Fisher Scientific, EP0753)
Deoxynucleotide (dNTP) Solution Mix (NEB, N0447L)
T4 DNA ligase (NEB, M0202L).
10× T4 ligase buffer New England Biolabs, B0202S).
Proteinase K from Tritirachium album (20 mg/ml in H2O) (Sigma-Aldrich, P2308-MG).
Sodium Dodecyl Sulfate 20.
Phenylmethanesulfonyl fluoride (PMSF) (Sigma-Aldrich, P7626).
2-Propanol (IPA) (Sigma-Aldrich, 19516)
0.5M EDTA (Thermo Fisher Scientific, AM9260G)
· Tween 20 (Sigma-Aldrich, P9416-100ML).
Ficoll PM-400 (20.
KAPA HiFi HotStart ReadyMix (Fisher Scientific. NC0295239)·
AMPure XP (Beckman Coulter, A63880).
100% Ethanol (Sigma-Aldrich, 8.18760.2500).
Qubit dsDNA HS Assay Kit, (Thermo Fisher Scientific, Q32854)
Gel (E-gel/Flash gels)·
FlashGel DNA Cassettes (Lonza, 57031)
Dithiothreitol (DTT), 0.1M Solution (Thermo Fisher Scientific, 707265ML).
NEBNext High-Fidelity 2× PCR Master Mix (New England Biolabs, M0541L)
Glycerol (Thermo Fisher Scientific, 15514011)
· Unloaded Tn5 (Active Motif, 81286 or 81284).
TD buffer from Nextera kit.
10,000× SYBRgreen (Thermo Fisher Scientific, S7563)
TRIS-acetate buffer 0.2M, pH 7.8 (Bioworld. 40120265-2).
Potassium acetate (Sigma-Aldrich. 95843-100ML-F)
Magnesium acetate (Sigma-Aldrich, 63052-100ML)
Dimethylformamide (DMF) (Thermo Fisher Scientific. 20673A).
Protease Inhibitor Cocktail (Sigma-Aldrich. P8340)
· Quick-Load® Purple 100 bp DNA Ladder (NEB, NO551S)
Gel Loading Dye. Purple (6X) (NEB, B7024S).
Oligonucleotides (IDT)

### Example 2. Tn5-Mediated Single Cell DS

The present Example describes the use of Tn5-mediated single cell DS and the incorporation of a SDE by primer extension for the purposes of highly accurate and sensitive sequencing. DNA transposase technology was used to simultaneously fragment and attach sequencing adapters. In the present Example, the Tn5 transposase was used. Following the loading of the Tn5 transposase with Tn5 compatible adapters composed of the Tn5 Mosaic End and one of two single-stranded tails comprised of the sequencing primer binding site, UMI, optional sample index sequence, SDE region, and PCR primer binding site to form a transposome, 2 μL. 4 μL, 6 μL and 8 μL of Tn5 transposomes at 2 uM concentration was incubated with 60 ng of human genomic DNA from the commercially available HCT116 colorectal cancer cell line at 55C for 15 min. The 8 μL of Tn5 transposomes at 2 uM showed nearly complete genome fragmentation and adapter attachment, indicating this adapter design is functional. The post-transposed DNA with single-stranded tails was made fully double-stranded, thus forming a double-stranded UMI, by the addition of a commercially available mixture of Taq DNA polymerase and Vent polymerase (New England BiolabsR. Ipswitch MA) that is both strand displacing and deoxyuracil tolerant along with 200 nM dNTPs for 15 minutes at 72C. The SDE primers (not shown) were added to a concentration of 10 uM. The sample was heated to 95C for 2 minutes followed by a slow cooling to 60C over a 5-minute time period followed by heating to 72C for 15 minutes. The uracil containing portion of the adapter sequence was removed by the treatment with a commercially available mixture of uracil deglycosylase and endonuclease VIII (USER Enzyme; New England Biolabs®, Ipswitch MA) at 37 C for 15 minutes. The SDE primers and USER enzyme were removed by precipitating the sample DNA onto magnetic beads using a solution of 20% PEG6000 and 2M salt at a 1.2× vol/vol ratio. The SDE containing strands were PCR amplified with a commercially available high fidelity, dU intolerant polymerase (which prevents amplification of the original, non-SDE containing DNA strand) (Q5 DNA polymerse, New England BiolabsR, Ipswitch MA) to introduce flowcell primers for the purposes of sequencing on the IlluminaR sequencing platform. In the present Example, specific genomic regions were enriched with targeted hybridization capture using biotinylated DNA oligonucleotide probes specific to those regions using a commercially available target enrichment kit optimized for these oligonucleotide probes (TwinStrand BiosciencesR, Seattle WA). In the present Example, these regions are used to assess environmental and/or exposure-based mutagenesis. After targeted enrichment, the captured DNA was PCR amplified using a commercially available high-fidelity DNA polymerase (TwinStrand Biosciences R, Seattle WA) and sequenced on an IlluminaR MiSeq sequencer and devoting ˜15×10⁶ 75-cycle paired end reads with dual 16-cycle index reads that comprised the 5 bp UMI, 8 bp sample index, and 3 bp SDE sequences.

A custom bioinformatic pipeline was created to automate the analysis from raw FASTQ files to text files. This pipeline is similar to methods used for standard Duplex Sequencing analysis that have been previously published (Sanchez-Contreras et al. Nucleic Acids Res. 49 (19); 11103-11118, 2021, incorporated herein by reference in its entirety) but with the following several modifications; 1) The UMI and SDE sequences were parsed from the index reads and associated with the corresponding read sequence in a special user defined field in an unaligned BAM file; 2) Consensus making is performed differently. Specifically, complementary sequences derived from the same end of the same parental molecule are found in the same read end (i.e. Read 1 end or Read 2 end). Strand identification is achieved, not by the read end in which complementary sequences are located, but by the orientation of the SDE sequence in the user defined field in an unaligned bam file (e.g., Top strand orientation; TTT-GGG vs Bottom strand orientation; GGG-TTT)

It is considered that this same approach could be performed on an aligned BAM file.

In Tn5-mediated single cell DS, consensus making is executed by a custom Python scripted called Tn5ConsensusMaker.py, which takes all the reads that are derived from the same tag and have the same SDE sequence order, and compares the base called at each position and produces a single-stranded consensus sequence (SSCS) read. The SSCS reads for each pair of tags with opposite oriented SDE sequence order can then be compared position by position to create a double-stranded consensus sequence (DCS) read). Two FASTQ files were made containing the resulting SSCS reads and DCS reads. The resulting two FASTQ files for the DCS reads are then aligned against the human reference genome (hg38) using the publicly available program bwa-mem v0.7.419 with default parameters. Overlapping areas of read pairs are trimmed back using fgbio ClipOVerlappingReads. When not evaluating the aggregate distribution of variants along the read space, the 5′ portion of each read can be trimmed by 9 bases to remove the region corresponding to the single-stranded gap formed by Tn5 transposition with fgbio ClipBam. Next, sequences on the 3′ end of the reads corresponding to the Tn5 adapter sequences can be removed by the publicly available program clipAdapt. Lastly, the resulting BAM file can be filtered to remove reads not aligning to the genomic positions of interest using a BED file. The BED file can be easily created using the coordinates of the target hybridization probes. Variant calling on the final post-processed BAM file can be performed by the publicly available program VarDictJava and reported in VCF format. The VCF file can be filtered to remove inherited single nucleotide polymorphisms. Mutation frequencies are calculated by a custom-made Python script that uses the VCF file and BAM file to count both the number of variants detected and the number of nucleotides sequenced and then divides the number of variants by the number of sequenced nucleotides.

It is expected that the present Example can demonstrate that error correction is comparable to a conventional approach to Duplex Sequencing that entails fragmentation of gDNA by sonication, enzymatic end-repair to form blunt ends, the addition of a dA base to the 3′ ends of the blunt end molecule (i.e, dA-tailing), and the ligation of sequencing adapters containing a double-stranded UMI. It is also expected that the present Example can demonstrate the ability to detect mutations in a small amount of input DNA (˜60 ng) such that mutational signature analysis detects mismatch repair deficiency, which is known in the HCT116 cell line.

The present Example demonstrates how false mutations can be prevented by use of Tn5-mediated fragmentation because Tn5-mediated fragmentation results in an extremely well defined 9 bp single-strand region of the gDNA that is more damage prone and that can easily be ignored during bioinformatic processing. This is in contrast to physical shearing methods, such as sonication, that have been demonstrated to result in highly variable single-stranded regions and strand nicking (Xiong et al., Nucleic Acids Res. 50 (1); el; 2021). In the present Example, it is expected that excess apparent mutations only cluster in the known 9 bp single-stranded region.

### Non-Limiting Embodiments

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.

Embodiment 1. A method for preparing a sequencing library for duplex sequencing. the method comprising; a) introducing a sample index to cellular nucleic acid molecules of cells of a biological sample; b) introducing a cell index to the cells according to a method of cell indexing, the method of cell indexing comprising; a.dividing an initial pool of the cells into a plurality of aliquots, wherein cell membranes of the cells retain cellular nucleic acid molecules therein; b, contacting an aliquot of the plurality of aliquots with a corresponding aliquot-specific barcode nucleic acid such that the cellular nucleic acid molecules of the aliquot are ligated to the corresponding aliquot-specific barcode nucleic acid to form aliquot-barcoded cellular nucleic acid molecules within cells of the aliquot; c, pooling the plurality of aliquots of cells to form a subsequent pool, wherein the subsequent pool serves as the initial pool of the cells for a subsequent repetition of steps a . . , b., and c.; and d, repeating steps a., b., and c, until most cells or every cell of the biological sample contains cellular nucleic acid molecules therein that are uniquely labeled according to the cell index; c) introducing a molecular index as a unique molecular identifier (UMI) to the cellular nucleic acid molecules; and d) providing a strand index as a strand-defining element (SDE) to the cellular nucleic acid molecules; wherein the sample index is for identification of a particular biological sample among biological samples, the cell index is for identification of a particular cell among cells, the molecular index is for identification of a particular cellular nucleic acid molecule among cellular nucleic acid molecules, and the strand index is for identification of a particular strand of the particular cellular nucleic acid molecule.

Embodiment 2. The method of Embodiment 1, wherein prior to introducing a sample index or cell index, the method further comprises; i, permeabilizing the cells, wherein the cell membranes of the cells retain cellular nucleic acid molecules therein; ii, depleting the cells of nucleosomes; and iii, fragmenting genomes of the cells to form at least part of the cellular nucleic acid molecules.

Embodiment 3. The method of any one of Embodiments 1-2, wherein the sample index is introduced to the cellular nucleic acid molecules by; contacting the biological sample comprising the cellular nucleic acid molecules with a corresponding sample-specific barcode nucleic acid such that the cellular nucleic acid molecules of the biological sample are ligated to the corresponding sample-specific barcode nucleic acid to form sample-barcoded cellular nucleic acid molecules within cells of the sample.

Embodiment 4. The method of any one of Embodiments 1-3, wherein the UMI is introduced to the cellular nucleic acid molecules by; including the UMI in a corresponding aliquot-specific barcode nucleic acid of step b); and converting the UMI to a double-stranded form of the UMI via polymerase extension.

Embodiment 5. The method of any one of Embodiments I-4, further comprising; ligating sequencing adaptors to the cellular nucleic acid molecules for duplex sequencing.

Embodiment 6. The method of any one of Embodiments 1-5, wherein the SDE is introduced to the cellular nucleic acid molecules by; including a base pair mismatch in a corresponding aliquot-specific barcode nucleic acid of step b).

Embodiment 7. The method of any one of Embodiments 1-5, wherein the SDE is introduced to the cellular nucleic acid molecules by; annealing a SDE primer to the aliquot-barcoded cellular nucleic acid molecules of step b), wherein the SDE primer comprises a base pair mismatch relative to a reference strand of the aliquot-barcoded cellular nucleic acid molecules; extending, with a polymerase, from the SDE primer to form a mutant strand complexed with the reference strand, wherein the mutant strand comprises the base pair mismatch; and amplifying, with PCR, the cellular nucleic acid molecules to convert the base pair mismatch to the SDE.

Embodiment 8. The method of any one of Embodiments 1-7, wherein the cellular nucleic acid molecules comprise genomic DNA (gDNA) fragments.

Embodiment 9. The method of any one of Embodiments 1-7, wherein the cellular nucleic acid molecules comprise complementary DNA (cDNA) produced by reverse transcription of cellular RNA.

Embodiment 10. The method of any one of Embodiments 1-9, wherein low-frequency biological variants are distinguishable from low-frequency technical variants with duplex sequencing.

Embodiment 11. A method for preparing a sequencing library for duplex sequencing. the method comprising; a) covalently attaching. with a transposase, a barcode nucleic acid molecule to cellular nucleic acid molecules of cells of a biological sample to form barcoded cellular nucleic acid molecules; and b) introducing a strand index as a strand-defining element (SDE) to the cellular nucleic acid molecules.

Embodiment 12. The method of Embodiment 11, further comprising; ligating sequencing adaptors to the barcoded cellular nucleic acid molecules to configure the barcoded cellular nucleic acid molecules for duplex sequencing.

Embodiment 13. The method of any one of Embodiments 11-12, further comprising. before step a), introducing a cell index to the cells according to a method of cell indexing, such that cellular nucleic acid molecules derived from a particular cell can be identified.

Embodiment 14. The method of Embodiment 13, wherein the method of cell indexing comprises; a, dividing an initial pool of the cells into a plurality of aliquots, wherein cell membranes of the cells retain cellular nucleic acid molecules therein; b, contacting an aliquot of the plurality of aliquots with a corresponding aliquot-specific barcode nucleic acid such that the cellular nucleic acid molecules of the aliquot are ligated to the corresponding aliquot-specific barcode nucleic acid to form aliquot-barcoded cellular nucleic acid molecules within cells of the aliquot; c, pooling the plurality of aliquots of cells to form a subsequent pool, wherein the subsequent pool serves as the initial pool of the cells for a subsequent repetition of steps a . . , b., and c.; and d, repeating steps a . . , b. . , and c, until most cells or every cell of the biological sample contains cellular nucleic acid molecules therein that are uniquely labeled according to the cell index.

Embodiment 15. The method of any one of Embodiments 11-14, wherein nucleic acid molecules of the sequencing library comprise; a sample index, a cell index, a molecular index, and a strand index; wherein the sample index is for identification of a particular biological sample among biological samples, the cell index is for identification of a particular cell among cells, the molecular index is for identification of a particular cellular nucleic acid molecule among cellular nucleic acid molecules, and the strand index is for identification of a particular strand of the particular cellular nucleic acid molecule.

Embodiment 16. The method of any one of Embodiments 14-15, wherein the SDE is introduced to the cellular nucleic acid molecules by; including a base pair mismatch in a corresponding aliquot-specific barcode nucleic acid of step b.; and amplifying. with polymerase chain reaction (PCR), the cellular nucleic acid molecules to convert the base pair mismatch to the SDE.

Embodiment 17. The method of any one of Embodiments 14-15, wherein the SDE is introduced to the cellular nucleic acid molecules by; annealing a SDE primer to the barcoded cellular nucleic acid molecules of step a), wherein the SDE primer comprises a base pair mismatch relative to a reference strand of the barcoded cellular nucleic acid molecules; extending, with a polymerase, from the SDE primer to form a mutant strand complexed with the reference strand, wherein the mutant strand comprises the base pair mismatch; and amplifying. with PCR, the cellular nucleic acid molecules to convert the base pair mismatch to the SDE.

Embodiment 18. The method of any one of Embodiments 11-17, wherein the cellular nucleic acid molecules comprise genomic DNA (gDNA) fragments.

Embodiment 19. The method of any one of Embodiments 11-17, wherein the cellular nucleic acid molecules comprise complementary DNA (cDNA) produced by reverse transcription of cellular RNA.

Embodiment 20. The method of any one of Embodiments 11-19, wherein low-frequency biological variants are distinguishable from low-frequency technical variants.

Embodiment 21. A method for preparing a sequencing library for duplex sequencing, the method comprising; a) covalently attaching. with a transposase, first and second sequencing adaptors to cellular nucleic acid molecules of cells of a biological sample to form adaptor-labeled cellular nucleic acid molecules; and b) introducing a strand index as a strand-defining element (SDE) to the cellular nucleic acid molecules, wherein the SDE is an orientation of the sequencing adaptors of the adaptor-labeled cellular nucleic acid molecules with respect to a 5′ end or a 3′ end of the adaptor-labeled cellular nucleic acid molecules.

Embodiment 22. The method of Embodiment 21, wherein the SDE is introduced by; annealing a first barcode nucleic acid molecule with a 5′ single-strand overhang to a 3′ end of the first sequencing adaptor and extending. with a polymerase, from a 3′ end of the first barcode nucleic acid molecule; annealing a second barcode nucleic acid molecule with a 5′ single-strand overhang to a 3′ end of the second sequencing adaptor and extending, with a polymerase, from a 3′ end of the second barcode nucleic acid molecule; wherein a polynucleotide sequence of the first barcode nucleic acid molecule is different from a polynucleotide sequence of the second barcode nucleic acid molecule; and amplifying, with PCR, the cellular nucleic acid molecules to form the sequencing library.

Embodiment 23. A method for preparing a sequencing library for duplex sequencing, the method comprising; a) covalently attaching. with a transposase, a first sequencing adaptor to cellular nucleic acid molecules of cells of a biological sample to form adaptor-labeled cellular nucleic acid molecules, wherein the first sequencing adaptor is attached to a single strand of a mosaic end (ME) element by a deoxyuracil (dU) residue; and b) introducing a strand index as a strand-defining element (SDE) to the cellular nucleic acid molecules, wherein the SDE is a distance from a barcode of the adaptor-labeled cellular nucleic acid molecules to a sequence of the cellular nucleic acid molecules.

Embodiment 24. The method of Embodiment 23, wherein the SDE is introduced by; gap-filling with a dU-intolerant polymerase; annealing a first barcode nucleic acid molecule comprising a second sequencing adaptor to the ME and extending, with a polymerase, from a 3′ end of the first barcode nucleic acid molecule and from a 3′ end of the ME to form first-barcoded nucleic acid molecules; annealing a second barcode nucleic acid molecule to a 3′ end of the first-barcoded nucleic acid molecules and extending, with a polymerase, from a 3′ end of the second barcode nucleic acid molecule to form first- and second-barcoded nucleic acid molecules; wherein a polynucleotide sequence of the first barcode nucleic acid molecule is different from a polynucleotide sequence of the second barcode nucleic acid molecule; and amplifying, with PCR, the cellular nucleic acid molecules to form the sequencing library.

Embodiment 25. A method of generating an error-corrected sequence read of a double-stranded genomic DNA (gDNA) material in a cell-specific or cell-identifiable manner, the method comprising; accessing cellular nucleic acid material within cells and/or cellular organelles, wherein the cellular nucleic acid material comprises the double-stranded gDNA material and double-stranded cDNA material derived from RNA within the cells and/or cellular organelles; indexing the cellular nucleic acid material thereby forming indexed-target nucleic acid complexes having a first population of complexes comprising indexed-target gDNA complexes and a second population of complexes comprising indexed-target cDNA complexes, wherein each indexed-target nucleic acid complex in a plurality of the indexed-target nucleic acid complexes comprises (a) a cell index that identifies the target nucleic acid material as originating from a particular cell among a population of cells, and (b) a molecular index comprising a UMI sequence that identifies the indexed-target nucleic acid complex among the first and second populations of complexes; providing an SDE for the indexed-target nucleic acid complexes of the first population, wherein the SDE identifies a particular strand of a particular indexed-target nucleic acid complex; for one or more indexed-target nucleic acid complexes in the first population-amplifying each strand of the indexed-target nucleic acid complex to produce a plurality of first strand amplicons and a plurality of second strand amplicons; sequencing one or more of the first strand amplicons and one or more of the second strand amplicons to produce one or more first strand sequence reads and one or more second strand sequence reads; confirming the presence of at least one first strand sequence read and at least one second strand sequence read; and comparing the at least one first strand sequence read with the at least one second strand sequence read and generating an error-corrected sequence read of the double-stranded target nucleic acid material by—discounting nucleotide positions that do not agree; or removing compared first and second strand sequence reads having one or more nucleotide positions where the compared first and second strand sequence reads are non-complementary.

Embodiment 26. The method of Embodiment 25, wherein for one or more indexed-target nucleic acid complexes in the second population, the method further comprises; amplifying the indexed-target cDNA complex to produce a plurality of indexed-target cDNA complex amplicons; sequencing one or more of the indexed-target cDNA complex amplicons to produce one or more indexed-target cDNA complex sequence reads; and grouping the indexed-target cDNA complex sequence reads into a cell-specific family using the cell index.

Embodiment 27. The method of Embodiment 26, further comprising; generating a transcriptome profile for one or more cell-specific families; and determining a cell type for the one or more cell-specific families based on the transcriptome profile.

Embodiment 28. The method of Embodiment 27, further comprising determining the cell type associated with the error-corrected sequence read of the double-stranded target nucleic acid material.

Embodiment 29. The method of Embodiment 27, further comprising identifying the cell and/or cellular organelle from which the error-corrected sequence read of the double-stranded target nucleic acid material is derived.

Embodiment 30. The method of any one of Embodiments 25-29, further comprising; aligning the error-corrected sequence read of the double-stranded target nucleic acid material to a reference sequence; and determining if there is a variant present in the double-stranded target nucleic acid material by comparing the error-corrected sequence read to the reference sequence.

Embodiment 31. The method of Embodiment 30, further comprising identifying the cell and/or cellular organelle comprising the variant.

Embodiment 32. The method of any one of Embodiments 30-31, wherein determining if there is a variant present comprises determining the presence of a plurality of variants present in the double-stranded target nucleic acid material.

Embodiment 33. The method of any one of Embodiments 30-32, wherein the cells and/or cellular organelles are derived from a biological sample obtained from a subject, and wherein the method further comprises; classifying the variant as a disease-associated mutation; and determining the presence or absence of a disease state within the subject based on the cell-type of the cell and/or cellular organelle from which the disease-associated mutation was derived.

Embodiment 34. The method of any one of Embodiments 32-33, wherein the cells and/or cellular organelles are derived from a biological sample obtained from a subject, and wherein the method further comprises; classifying one or more combinations of the plurality of variants as a disease-associated mutation combination; and determining the presence or absence of a disease state within the subject based on the disease-associated mutation combination within a cell.

Embodiment 35. The method of any one of Embodiments 33-34, wherein the disease state is cancer.

Embodiment 36. The method of Embodiments 33-34, wherein the disease state is an increased risk of developing cancer.

Embodiment 37. The method of any one of Embodiments 25-36, wherein at least one cell is an abnormal cell.

Embodiment 38. The method of any one of Embodiments 25-37, further comprising selectively enriching the first population of the indexed-target nucleic acid complexes for one or more targeted genomic regions prior to sequencing to provide a plurality of enriched indexed-target nucleic acid complexes in the first population.

Embodiment 39. The method of any one of Embodiments 25-38, further comprising providing cells and/or cellular organelles, wherein the cells and/or cellular organelles have been fixed and permeabilized prior to providing.

Embodiment 40. The method of any one of Embodiments 25-39, further comprising fragmenting genomic DNA using one or more enzymes that cut double-stranded nucleic acids.

Embodiment 41. The method any one of Embodiments 25-40, wherein indexing the cellular nucleic acid material comprises using combinatorial cellular indexing steps to add a unique combination of index sequences to the cellular nucleic acid material.

Embodiment 42. The method of any one of Embodiments 25-39, further comprising integrating adapter sequences into the double-stranded gDNA material via a transposase mediated event.

Embodiment 43. The method of any one of Embodiments 25-42, wherein the SDE is a base pair mismatch flanked by complementary sequences.

Embodiment 44. The method of any one of Embodiments 26-39 and 42, wherein the SDE is an orientation of one or more barcode sequences relative to the cellular nucleic acid material of the indexed-target nucleic acid complex.

Embodiment 45. The method of any one of Embodiments 25-44, wherein prior to the indexing the step, the method further comprises reverse transcribing RNA to cDNA.

Embodiment 46. A method of identifying a DNA variant in a cell within a population of cells, the method comprising; providing a population of cells from a biological sample; accessing cellular nucleic acid material within cells of the population, wherein the cellular nucleic acid material comprises double-stranded gDNA material and single-stranded RNA material within the cells; indexing the double-stranded gDNA material to generate indexed-target gDNA complexes, wherein each indexed-target gDNA complex in a plurality of the indexed-target gDNA complexes comprises (a) a cell index that identifies the target gDNA material as originating from a particular cell among the population of cells, and (b) a UMI that identifies the indexed-target gDNA complex among the plurality of the indexed-target gDNA complexes; providing an SDE for the indexed-target gDNA complexes, wherein the SDE identifies a particular strand of a particular indexed-target gDNA complex; reverse transcribing the single-stranded RNA material within the cells to generate double stranded cDNA material; indexing the double-stranded cDNA material to generate indexed-target cDNA complexes, wherein each indexed-target cDNA complex in a plurality of the indexed-target cDNA complexes comprises (a) the cell index that identifies the target cDVA material originating from the same particular cell among the population of cells, and (b) a UMI that identifies the indexed-target cDNA complex among the plurality of the indexed-target cDNA complexes and indexed-target gDNA complexes; for one or more indexed-target gDNA complexes in the plurality of the indexed-target gDNA complexes-amplifying each of a first strand and a second strand of the indexed-target gDNA complex. resulting in each strand generating a distinct. yet related, set of amplified indexed-target gDNA products; sequencing each of a plurality of first strand indexed-target gDNA products and a plurality of second strand indexed-target nucleic acid products; confirming the presence of at least one sequence read from each strand of the indexed-target gDNA complex; and comparing the at least one sequence read obtained from the first strand with the at least one sequence read obtained from the second strand to form a consensus sequence read of the indexed-target gDNA complex having only nucleotide bases at which the sequence of both strands of the indexed-target gDNA complex are in agreement, such that a variant occurring at a particular position in the consensus sequence read is identified as a true DNA variant.

Embodiment 47. The method of Embodiment 46, wherein confirming the presence of at least one sequence read from each strand comprises identifying the presence of a first strand sequence read and a second strand sequence read using the cell index, the UMI and the SDE.

Embodiment 48. The method of any one of Embodiments 46-47, wherein for one or more indexed-target cDNA complexes in the plurality of the indexed-target cDNA complexes, the method further comprises; amplifying the indexed-target cDNA complex to produce a set of amplified indexed-target cDNA products; sequencing one or more of the amplified indexed-target cDNA products to generate one or more indexed-target cDNA complex sequence reads; and grouping the indexed-target cDNA complex sequence reads that share the same cell-index into a cell-specific family.

Embodiment 49. The method of any one of Embodiments 26-48, wherein grouping the indexed-target cDNA complex sequence reads comprises grouping indexed-target cDNA complex sequence reads that share the same cell-index and UMI.

Embodiment 50. The method of any one of Embodiments 48-49, further comprising; generating a transcriptome profile for one or more cell-specific families; and determining a cell type for the one or more cell-specific families based on the transcriptome profile.

Embodiment 51. The method of Embodiment 50, further comprising determining the cell type associated with the consensus sequence read of the double-stranded gDNA material.

Embodiment 52. The method of Embodiment 50, further comprising identifying the cell within the population of cells from which the consensus sequence read of the double-stranded gDNA material is derived.

Embodiment 53. The method of any one of Embodiments 26-52, wherein a variant occurring at a particular position in the consensus sequence read is identified as a true DNA variant, and is further determined by; aligning the consensus sequence read of the double-stranded gDNA material to a reference sequence; and determining if there is a variant present in the double-stranded gDNA material by comparing the consensus sequence read to the reference sequence.

Embodiment 54. The method of Embodiment 53, further comprising identifying the cell comprising the variant.

Embodiment 55. The method of any one of Embodiments 53-54, wherein determining if there is a variant present comprises determining the presence of a plurality of variants present in the double-stranded gDNA material.

Embodiment 56. The method of any one of Embodiments 53-55, wherein the biological sample is obtained from a subject, and wherein the method further comprises; classifying the variant as a disease-associated mutation; and determining the presence or absence of a disease state within the subject based on the cell-type of the cell from which the disease-associated mutation was derived.

Embodiment 57. The method of any one of Embodiments 53-55, wherein the biological sample is obtained from a subject, and wherein the method further comprises;

classifying one or more combinations of the plurality of variants as a disease-associated mutation combination; and determining the presence or absence of a disease state within the subject based on the disease-associated mutation combination within a cell.

Embodiment 58. The method of any one of Embodiments 56-57, wherein the disease state is cancer.

Embodiment 59. The method of any one of Embodiments 56-57, wherein the disease state is a cancer-like state or a pre-cancerous state.

Embodiment 60. The method of Embodiments 56-57, wherein the disease state is an increased risk of developing cancer.

Embodiment 61. The method of any one of Embodiments 26-60, wherein at least one cell is an abnormal cell.

Embodiment 62. The method of any one of Embodiments 53-61, wherein the variant comprises a functionally disruptive variant.

Embodiment 63. The method of any one of Embodiments 53-61, wherein the variant is a passenger mutation.

Embodiment 64. The method of any one of Embodiments 53-61, wherein the variant is a non-cancer driver variant.

Embodiment 65. The method of any one of Embodiments 53-62, further comprising determining a frequency of a cell comprising the variant among the population of cells.

Embodiment 66. The method of any one of Embodiments 56-65, wherein the disease-associated mutation or disease-associated mutation combination comprises a mutation in a tumor suppressor gene, an oncogene, a proto-oncogene, and/or a cancer driver gene.

Embodiment 67. The method of any one of Embodiments 26-66, further comprising selectively enriching one or more targeted genomic regions prior to sequencing to provide a plurality of enriched indexed-target gDNA complexes.

Embodiment 68. The method of any one of Embodiments 56-67, wherein the disease-associated mutation is in TP53.

Embodiment 69. The method of any one of Embodiments 56-67, wherein the disease-associated mutation is in HRAS, NRAS or KRAS.

Embodiment 70. The method of any one of Embodiments 56-67, wherein the disease-associated mutation is in ABL. ACC, BCR, BLCA, BRCA, CESC, CHOL, COAD, DLBC. DNMT3A, EGFR, ESCA, GBM, HNSC, KICH, KIRC, KIRP, LAML, LGG, LIHC, LUAD, LUSC, MESO, OV. PAAD, PCPG, PI3K, PIK3CA, PRAD, PTEN. READ, SARC, SKCM, STAD. TGCT. THCA. THYM, UCEC, UCS, and/or UVM.

Embodiment 71. The method of any one of Embodiments 67-70, further comprising determining a variant frequency of the variant among the plurality of enriched indexed-target gDNA complexes.

Embodiment 72. The method of any one of Embodiments 26-71, wherein the population of cells is derived from a human patient.

Embodiment 73. The method of Embodiment 72, wherein the population of cells is obtained from tissue, from circulating cells in blood, from other bodily fluids, shedding tumors, and/or from a biopsy.

Embodiment 74. The method of Embodiment 73, wherein the other bodily fluids comprise uterine lavage fluid, urine, or gastric lavage fluid.

Embodiment 75. The method of any one of Embodiments 26-71, wherein the population of cells is derived from a transplanted tissue.

Embodiment 76. A kit configured for error corrected duplex sequencing of double-stranded nucleic acids to characterize a cell within a population of cells, the kit comprising at least one set of combinatorial cell indexing oligonucleotides, wherein at least a subset of the oligonucleotides comprises a UMI and an SDE for error corrected duplex sequencing.

Embodiment 77. The kit of Embodiment 76, further comprising an endonuclease or mixture of endonucleases configured to fragment gDNA in a cell.

Embodiment 78. The kit of any one of Embodiments 76 and 77, further comprising a ligase and reverse transcriptase.

Embodiment 79. The kit of any one of Embodiments 76-78, further comprising instructions on methods of use of the kit in conducting single cell duplex sequencing.

Embodiment 80. A non-transitory computer-readable storage medium having instructions stored thereon which, when executed by at least one processor, cause the at least one processor to perform a method for providing duplex sequencing data for double-stranded nucleic acid molecules in a cell from a biological sample, the method comprising; receiving raw sequence data from a user computing device; creating a sample-specific data set comprising a plurality of raw sequence reads derived from a plurality of nucleic acid molecules in the sample; grouping sequence reads from families representing an original double-stranded nucleic acid molecule, wherein the grouping is based on a shared unique identifier (UMI) sequence; comparing a first strand sequence read and a second strand sequence read from an original double-stranded nucleic acid molecule to identify one or more correspondences between the first and second strand sequences reads; providing duplex sequencing data for the double-stranded nucleic acid molecules in the sample; and grouping duplex consensus sequences into cell families representing an original cell, wherein the grouping is based on a shared cell index sequence.

Embodiment 81. The non-transitory computer-readable storage medium of Embodiment 80, the method further comprising; receiving cDNA raw sequencing reads from the user computing device; grouping sequence reads from families representing an original double-stranded cDNA molecule, wherein grouping is based on a shared UMI sequence; grouping cDNA sequence reads into the cell families representing an original cell, wherein the grouping is based on a shared cell index sequence; generating a transcriptome profile from the grouped cDNA sequence reads in the cell families; and determining a cell profile from the transcriptome profile for the cell families.

Embodiment 82. The non-transitory computer-readable storage medium of Embodiment 81, the method further comprising identifying one or more variants in a cell in the biological sample, wherein the method further comprises; comparing the duplex sequencing data to reference sequence information; identifying one or more sequence variations between the duplex sequencing data and the reference sequence information at one or more genomic loci; correlating the one or more variants identified in a cell family with the cell profile of the cell family; and providing cell-specific variant data.

### CONCLUSION

The complete disclosure of all patents, patent applications, and publications, and electronically available material including, for instance, nucleotide sequence submissions in, e.g., . GenBank and RefSeq, and amino acid sequence submissions in, e.g., SwissProt, PIR. PRF. PDB, and translations from annotated coding regions in GenBank and RefSeq cited herein, are incorporated by reference in their entirety. Supplementary materials referenced in publications (such as supplementary tables, supplementary figures, supplementary materials and methods. and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore. while advantages associated with certain embodiments of the disclosure have been described in the context of these embodiments, other embodiments may also exhibit such advantages, and not all embodiments need necessarily exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims. the words comprise′, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components. molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description. While illustrative embodiments have been illustrated and described, it will be appreciated that various changes can be made therein without departing from the spirit and scope of the invention.

1. A method for preparing a sequencing library for duplex sequencing, the method comprising;
a) introducing a sample index to cellular nucleic acid molecules of cells of a biological sample;
b) introducing a cell index to the cells according to a method of cell indexing, the method of cell indexing comprising;
a, dividing an initial pool of the cells into a plurality of aliquots, wherein cell membranes of the cells retain cellular nucleic acid molecules therein;
b, contacting an aliquot of the plurality of aliquots with a corresponding aliquot-specific barcode nucleic acid such that the cellular nucleic acid molecules of the aliquot are ligated to the corresponding aliquot-specific barcode nucleic acid to form aliquot-barcoded cellular nucleic acid molecules within cells of the aliquot;
c, pooling the plurality of aliquots of cells to form a subsequent pool, wherein the subsequent pool serves as the initial pool of the cells for a subsequent repetition of steps a., b., and c.; and
d, repeating steps a., b., and c, until most cells or every cell of the biological sample contains cellular nucleic acid molecules therein that are uniquely labeled according to the cell index;
c) introducing a molecular index as a unique molecular identifier (UMI) to the cellular nucleic acid molecules; and
d) providing a strand index as a strand-defining element (SDE) to the cellular nucleic acid molecules;
wherein the sample index is for identification of a particular biological sample among biological samples, the cell index is for identification of a particular cell among cells, the molecular index is for identification of a particular cellular nucleic acid molecule among cellular nucleic acid molecules, and the strand index is for identification of a particular strand of the particular cellular nucleic acid molecule.

2. (canceled)

3. The method of claim 1, wherein the sample index is introduced to the cellular nucleic acid molecules by;
contacting the biological sample comprising the cellular nucleic acid molecules with a corresponding sample-specific barcode nucleic acid such that the cellular nucleic acid molecules of the biological sample are ligated to the corresponding sample-specific barcode nucleic acid to form sample-barcoded cellular nucleic acid molecules within cells of the sample; and/or wherein the SDE is introduced to the cellular nucleic acid molecules by;
annealing a SDE primer to the aliquot-barcoded cellular nucleic acid molecules of step b), wherein the SDE primer comprises a base pair mismatch relative to a reference strand of the aliquot-barcoded cellular nucleic acid molecules;
extending, with a polymerase, from the SDE primer to form a mutant strand complexed with the reference strand, wherein the mutant strand comprises the base pair mismatch; and
amplifying, with PCR, the cellular nucleic acid molecules to convert the base pair mismatch to the SDE.

4-10. (canceled)

11. A method for preparing a sequencing library for duplex sequencing, the method comprising;
a) covalently attaching, with a transposase, a barcode nucleic acid molecule to cellular nucleic acid molecules of cells of a biological sample to form barcoded cellular nucleic acid molecules; and
b) introducing a strand index as a strand-defining element (SDE) to the cellular nucleic acid molecules.

12. (canceled)

13. The method of claim 11, further comprising ligating sequencing adaptors to the barcoded cellular nucleic acid molecules to configure the barcoded cellular nucleic acid molecules for duplex sequencing, and/or
further comprising, before step a), introducing a cell index to the cells according to a method of cell indexing, such that cellular nucleic acid molecules derived from a particular cell can be identified, and/or
wherein the SDE is introduced to the cellular nucleic acid molecules by;
annealing a SDE primer to the barcoded cellular nucleic acid molecules of step a), wherein the SDE primer comprises a base pair mismatch relative to a reference strand of the barcoded cellular nucleic acid molecules;
extending, with a polymerase, from the SDE primer to form a mutant strand complexed with the reference strand, wherein the mutant strand comprises the base pair mismatch; and
amplifying, with PCR, the cellular nucleic acid molecules to convert the base pair mismatch to the SDE.

14-20. (canceled)

21. A method for preparing a sequencing library for duplex sequencing, the method comprising;
a) covalently attaching, with a transposase, first and second sequencing adaptors to cellular nucleic acid molecules of cells of a biological sample to form adaptor-labeled cellular nucleic acid molecules; and
b) introducing a strand index as a strand-defining element (SDE) to the cellular nucleic acid molecules, wherein the SDE is an orientation of the sequencing adaptors of the adaptor-labeled cellular nucleic acid molecules with respect to a 5′ end or a 3′ end of the adaptor-labeled cellular nucleic acid molecules.

22. (canceled)

23. A method for preparing a sequencing library for duplex sequencing, the method comprising;
a) covalently attaching, with a transposase, a first sequencing adaptor to cellular nucleic acid molecules of cells of a biological sample to form adaptor-labeled cellular nucleic acid molecules, wherein the first sequencing adaptor is attached to a single strand of a mosaic end (ME) element by a deoxyuracil (dU) residue; and
b) introducing a strand index as a strand-defining element (SDE) to the cellular nucleic acid molecules, wherein the SDE is a distance from a barcode of the adaptor-labeled cellular nucleic acid molecules to a sequence of the cellular nucleic acid molecules.

24. (canceled)

25. A method of generating an error-corrected sequence read of a double-stranded genomic DNA (gDNA) material in a cell-specific or cell-identifiable manner, the method comprising;
accessing cellular nucleic acid material within cells and/or cellular organelles, wherein the cellular nucleic acid material comprises the double-stranded gDNA material and double-stranded cDNA material derived from RNA within the cells and/or cellular organelles;
indexing the cellular nucleic acid material thereby forming indexed-target nucleic acid complexes having a first population of complexes comprising indexed-target gDNA complexes and a second population of complexes comprising indexed-target cDNA complexes, wherein each indexed-target nucleic acid complex in a plurality of the indexed-target nucleic acid complexes comprises (a) a cell index that identifies the target nucleic acid material as originating from a particular cell among a population of cells, and (b) a molecular index comprising a UMI sequence that identifies the indexed-target nucleic acid complex among the first and second populations of complexes;
providing an SDE for the indexed-target nucleic acid complexes of the first population, wherein the SDE identifies a particular strand of a particular indexed-target nucleic acid complex;
for one or more indexed-target nucleic acid complexes in the first population
amplifying each strand of the indexed-target nucleic acid complex to produce a plurality of first strand amplicons and a plurality of second strand amplicons;
sequencing one or more of the first strand amplicons and one or more of the second strand amplicons to produce one or more first strand sequence reads and one or more second strand sequence reads;
confirming the presence of at least one first strand sequence read and at least one second strand sequence read; and
comparing the at least one first strand sequence read with the at least one second strand sequence read and generating an error-corrected sequence read of the double-stranded target nucleic acid material by
discounting nucleotide positions that do not agree; or
removing compared first and second strand sequence reads having one or more nucleotide positions where the compared first and second strand sequence reads are non-complementary,
optionally wherein the SDE is a base pair mismatch flanked by complementary sequences.

26. The method of claim 25, wherein for one or more indexed target nucleic acid complexes in the second population, the method further comprises;
amplifying the indexed-target cDNA complex to produce a plurality of indexed-target cDNA complex amplicons;
sequencing one or more of the indexed-target cDNA complex amplicons to produce one or more indexed-target cDNA complex sequence reads; and
grouping the indexed-target cDNA complex sequence reads into a cell-specific family using the cell index;
optionally wherein the SDE is an orientation of one or more barcode sequences relative to the cellular nucleic acid material of the indexed-target nucleic acid complex;
optionally wherein a variant occurring at a particular position in the consensus sequence read is identified as a true DNA variant, and is further determined by; aligning the consensus sequence read of the double-stranded gDNA material to a reference sequence; and determining if there is a variant present in the double-stranded gDNA material by comparing the consensus sequence read to the reference sequence; and
optionally wherein the population of cells is derived from a human patient.

27-37. (canceled)

38. The method of claim 25, further comprising;
selectively enriching the first population of the indexed-target nucleic acid complexes for one or more targeted genomic regions prior to sequencing to provide a plurality of enriched indexed-target nucleic acid complexes in the first population;
providing cells and/or cellular organelles, wherein the cells and/or cellular organelles have been fixed and permeabilized prior to providing;
fragmenting genomic DNA using one or more enzymes that cut double-stranded nucleic acids;
integrating adapter sequences into the double-stranded gDNA material via a transposase mediated event; or
any combination thereof.

39-45. (canceled)

46. A method of identifying a DNA variant in a cell within a population of cells, the method comprising;
providing a population of cells from a biological sample;
accessing cellular nucleic acid material within cells of the population, wherein the cellular nucleic acid material comprises double-stranded gDNA material and single-stranded RNA material within the cells;
indexing the double-stranded gDNA material to generate indexed-target gDNA complexes, wherein each indexed-target gDNA complex in a plurality of the indexed-target gDNA complexes comprises (a) a cell index that identifies the target gDNA material as originating from a particular cell among the population of cells, and (b) a UMI that identifies the indexed-target gDNA complex among the plurality of the indexed-target gDNA complexes;
providing an SDE for the indexed-target gDNA complexes, wherein the SDE identifies a particular strand of a particular indexed-target gDNA complex;
reverse transcribing the single-stranded RNA material within the cells to generate double stranded cDNA material;
indexing the double-stranded cDNA material to generate indexed-target cDNA complexes, wherein each indexed-target cDNA complex in a plurality of the indexed-target cDNA complexes comprises (a) the cell index that identifies the target cDNA material originating from the same particular cell among the population of cells, and (b) a UMI that identifies the indexed-target cDNA complex among the plurality of the indexed-target cDNA complexes and indexed-target gDNA complexes;
for one or more indexed-target gDNA complexes in the plurality of the indexed-target gDNA complexes
amplifying each of a first strand and a second strand of the indexed-target gDNA complex, resulting in each strand generating a distinct, yet related, set of amplified indexed-target gDNA products;
sequencing each of a plurality of first strand indexed-target gDNA products and a plurality of second strand indexed-target nucleic acid products;
confirming the presence of at least one sequence read from each strand of the indexed-target gDNA complex; and
comparing the at least one sequence read obtained from the first strand with the at least one sequence read obtained from the second strand to form a consensus sequence read of the indexed-target gDNA complex having only nucleotide bases at which the sequence of both strands of the indexed-target gDNA complex are in agreement, such that a variant occurring at a particular position in the consensus sequence read is identified as a true DNA variant.

47. (canceled)

48. The method of claim 46, wherein for one or more indexed-target cDNA complexes in the plurality of the indexed-target cDNA complexes, the method further comprises;
amplifying the indexed-target cDNA complex to produce a set of amplified indexed-target cDNA products;
sequencing one or more of the amplified indexed-target cDNA products to generate one or more indexed-target cDNA complex sequence reads; and
grouping the indexed-target cDNA complex sequence reads that share the same cell-index into a cell-specific family;
optionally wherein confirming the presence of at least one sequence read from each strand comprises identifying the presence of a first strand sequence read and a second strand sequence read using the cell index, the UMI and the SDE.

49-55. (canceled)

56. The method of claim 26, wherein the biological sample is obtained from a subject, and wherein the method further comprises;
classifying the variant as a disease-associated mutation; and
determining the presence or absence of a disease state within the subject based on the cell-type of the cell from which the disease-associated mutation was derived.

57. (canceled)

58. The method of claim 56, wherein;
the disease state is cancer;
the disease state is a cancer-like state or a pre-cancerous state;
the disease state is an increased risk of developing cancer;
at least one cell is an abnormal cell;
the variant comprises a functionally disruptive variant;
the variant is a passenger mutation;
the variant is a non-cancer driver variant; or
any combination thereof.

59-65. (canceled)

66. The method of claim 56, wherein;
the disease-associated mutation or disease-associated mutation combination comprises a mutation in a tumor suppressor gene, an oncogene, a proto-oncogene, and/or a cancer driver gene;
the disease-associated mutation is in TP53;
the disease-associated mutation is in HRAS, NRAS or KRAS;
the disease-associated mutation is in ABL, ACC, BCR, BLCA, BRCA, CESC, CHOL, COAD, DLBC, DNMT3A, EGFR, ESCA, GBM, HNSC, KICH, KIRC, KIRP, LAML, LGG, LIHC, LUAD, LUSC, MESO, OV, PAAD, PCPG, PI3K, PIK3CA, PRAD, PTEN, READ, SARC, SKCM, STAD, TGCT, THCA, THYM, UCEC, UCS, and/or UVM; or
any combination thereof.

67. The method of claim 26, further comprising;
selectively enriching one or more targeted genomic regions prior to sequencing to provide a plurality of enriched indexed-target gDNA complexes, and
determining a variant frequency of the variant among the plurality of enriched indexed-target gDNA complexes.

68-72. (canceled)

73. The method of claim 26, wherein;
the population of cells is obtained from tissue, from circulating cells in blood, from other bodily fluids, shedding tumors, and/or from a biopsy, optionally wherein the other bodily fluids comprise uterine lavage fluid, urine, or gastric lavage fluid, and optionally wherein the population of cells is derived from a transplanted tissue.

74-75. (canceled)

76. A kit configured for error corrected duplex sequencing of double-stranded nucleic acids to characterize a cell within a population of cells, the kit comprising at least one set of combinatorial cell indexing oligonucleotides, wherein at least a subset of the oligonucleotides comprises a UMI and an SDE for error corrected duplex sequencing.

77. The kit of claim 76, further comprising;
an endonuclease or mixture of endonucleases configured to fragment gDNA in a cell;
a ligase and reverse transcriptase;
instructions on methods of use of the kit in conducting single cell duplex sequencing; or
any combination thereof.

78-79. (canceled)

80. A non-transitory computer-readable storage medium having instructions stored thereon which, when executed by at least one processor, cause the at least one processor to perform a method for providing duplex sequencing data for double-stranded nucleic acid molecules in a cell from a biological sample, the method comprising;
receiving raw sequence data from a user computing device;
creating a sample-specific data set comprising a plurality of raw sequence reads derived from a plurality of nucleic acid molecules in the sample;
grouping sequence reads from families representing an original double-stranded nucleic acid molecule, wherein the grouping is based on a shared unique identifier (UMI) sequence;
comparing a first strand sequence read and a second strand sequence read from an original double-stranded nucleic acid molecule to identify one or more correspondences between the first and second strand sequences reads;
providing duplex sequencing data for the double-stranded nucleic acid molecules in the sample; and
grouping duplex consensus sequences into cell families representing an original cell, wherein the grouping is based on a shared cell index sequence.

81. The non-transitory computer-readable storage medium of claim 80, the method further comprising;
receiving cDNA raw sequencing reads from the user computing device;
grouping sequence reads from families representing an original double-stranded cDNA molecule, wherein grouping is based on a shared UMI sequence;
grouping cDNA sequence reads into the cell families representing an original cell, wherein the grouping is based on a shared cell index sequence;
generating a transcriptome profile from the grouped cDNA sequence reads in the cell families; and
determining a cell profile from the transcriptome profile for the cell families;
optionally wherein the method further comprises identifying one or more variants in a cell in the biological sample, optionally wherein the method further comprises;
comparing the duplex sequencing data to reference sequence information;
identifying one or more sequence variations between the duplex sequencing data and the reference sequence information at one or more genomic loci;
correlating the one or more variants identified in a cell family with the cell profile of the cell family; and
providing cell-specific variant data.

82. (canceled)


SEQUENCE LISTING (13 sequences)

SEQ ID NO: 1 - DNA, 14 bp, synthetic construct
  source 1..14: mol_type = other DNA
agcagccgtcgcag

SEQ ID NO: 2 - DNA, 15 bp, synthetic construct
  source 1..15: mol_type = other DNA
gtctcgtgggctcgg

SEQ ID NO: 3 - DNA, 14 bp, synthetic construct
  source 1..14: mol_type = other DNA
tcgtcggcagcgtc

SEQ ID NO: 4 - DNA, 15 bp, synthetic construct
  source 1..15: mol_type = other DNA
gtctcgtgggctcgg

SEQ ID NO: 5 - DNA, 16 bp, synthetic construct
  source 1..16: mol_type = other DNA
  modified_base 1: mod_base = OTHER; note = deoxyuracil (dU)
nggctcgggtgctctg

SEQ ID NO: 6 - DNA, 35 bp, synthetic construct
  source 1..35: mol_type = other DNA
accgagcccacgagacaacacgatttttaaggcga

SEQ ID NO: 7 - DNA, 34 bp, synthetic construct
  source 1..34: mol_type = other DNA
ttgtgctagggattccgctgcttacgagaccgga

SEQ ID NO: 8 - DNA, 33 bp, synthetic construct
  source 1..33: mol_type = other DNA
tcgtcggcagcgtcagatgtgtataagagacag

SEQ ID NO: 9 - DNA, 17 bp, synthetic construct
  source 1..17: mol_type = other DNA
tctacacatattctctg

SEQ ID NO: 10 - DNA, 19 bp, synthetic construct
  source 1..19: mol_type = other DNA
ctgtctcttatacacatct

SEQ ID NO: 11 - DNA, 35 bp, synthetic construct
  source 1..35: mol_type = other DNA
  modified_base 20: mod_base = OTHER; note = deoxyuracil (dU)
gacagagaatatgtgtaganggctcgggtgctctg

SEQ ID NO: 12 - DNA, 35 bp, synthetic construct; identical to SEQ ID NO: 6
  source 1..35: mol_type = other DNA

SEQ ID NO: 13 - DNA, 23 bp, synthetic construct
  source 1..23: mol_type = other DNA
ttgtgctagggattccgctcgat